(19) United States
(12) Patent Application Publication
(10) Pub. No.: US 2024/0024497 A1
(43) Pub. Date: Jan. 25, 2024

(54) METHODS FOR THE SYNTHESIS OF PROTEIN-DRUG CONJUGATES

(71) Applicant: Cidara Therapeutics, Inc., San Diego, CA (US)

(72) Inventors: Allen BORCHARDT, San Diego, CA (US); Robert Michael HUGHES, San Diego, CA (US)

(21) Appl. No.: 18/019,855

(22) PCT Filed: Aug. 6, 2021
(86) PCT No.: PCT/US2021/045096

§ 371 Date: Feb. 6, 2023

Related U.S. Application Data
(60) Provisional application No. 63/159,781, filed on Mar. 11, 2021.
(60) Provisional application No. 63/154,514, filed on Feb. 26, 2021.
(60) Provisional application No. 63/062,377, filed on Aug. 6, 2020.

(51) Int. Cl. A61K 47/68; A61K 47/64

(52) U.S. Cl. CPC A61K 47/68; A61K 47/643; A61K 47/6803; A61K 47/6807

(57) ABSTRACT

The present invention relates to methods for the synthesis of conjugates useful for the treatment of viral infections, e.g., conjugates containing inhibitors of viral neuraminidase (e.g., zanamivir or an analog thereof) linked to an Fc domain monomer.

### SEQUENCE LISTING

The instant application contains a Sequence Listing which has been submitted electronically in ASCII format and is hereby incorporated by reference in its entirety. Said ASCII copy, created on Aug. 4, 2021, is named 50945-083WO1_Sequence_Listing_08_04_21_ST25 and is 33,939 bytes in size.

### BACKGROUND

This disclosure features methods for the synthesis of antiviral conjugates useful for the prevention or treatment of influenza and conditions related thereto.

The need for novel antiviral treatments for influenza is significant and especially critical in the medical field. Influenza virus, the causative agent of influenza, or the flu, is responsible for three to five million cases of severe illness annually, and approximately 500,000 deaths worldwide. While most people recover completely from influenza in about one to two weeks, others develop life-threatening complications, such as pneumonia. Thus, influenza can be deadly, especially for the young, old, or chronically ill. People with weak or compromised immune systems, such as people with advanced HIV infection or transplant patients, whose immune systems are medically suppressed to prevent transplant organ rejection, are at greater risk for complications relating to influenza. Pregnant women and young children are also at a high risk for complications. The development of antiviral treatments for influenza has been a continuing challenge. Drug-resistant strains have emerged to the most commonly used inhibitors.

Influenza antiviral agents largely target proteins presented on the surface of the influenza virus particle. The envelope of the influenza virus contains two immunodominant glycoproteins, hemagglutinin and neuraminidase, that play key roles in viral infection and spread. Hemagglutinin effects attachment of the virus to the host cell through its interaction with surface sialic acids, thereby initiating entry. Neuraminidase is an exo-glycosidase enzyme that cleaves sialic acids (terminal neuraminic acid residues) from glycan structures on the surface of infected host cells, releasing progeny viruses and allowing the spread of the virus from the host cell to uninfected surrounding cells. Inhibition of neuraminidase therefore serves as a pharmacological target for antiviral drugs. Viral neuraminidase inhibitors used to reduce viral spread have been identified, including oseltamivir (Tamiflu™), zanamivir (Relenza™), and peramivir (Rapivab™).

The utility of many therapeutics, such as small molecule agents and biologics such as peptides, polypeptides, and polynucleotides, suffer from inadequate serum half-lives. An effective way of increasing half-life and efficacy includes conjugating therapeutics (e.g., small molecule therapeutic agents and biologics such as peptides, polypeptides, and polynucleotides) to polypeptides to form, e.g., protein-drug conjugates. There is a need for convenient synthetic methods that permit the commercial scale production of such protein-drug conjugates. These approaches can be useful alternatives to existing synthetic methods and can achieve higher yield, higher purity, elimination of impurity (e.g., mutagenic impurity), reduced waste stream, or any combination of the above.

Accordingly new therapies for treating influenza and methods of synthesizing such therapies are needed.

### SUMMARY

The disclosure relates to methods and intermediates for synthesizing protein-drug conjugates. In particular, such conjugates contain a moiety (e.g., a monomer or a dimer of a moiety) that inhibits the influenza virus conjugated to an Fc monomer, Fc domain, or albumin protein. In some embodiments, the moiety is an inhibitor of a viral neuraminidase (e.g., zanamivir or an analog thereof). The neuraminidase inhibitor (e.g., zanamivir or an analog thereof) in the conjugates targets neuraminidase on the surface of the viral particle. The Fc monomers or Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of viral particles by immune cells and further enhancing the antiviral activity of the conjugates. The albumin or albumin-binding peptide may extend the half-life of the conjugate, for example, by binding of albumin to the recycling neonatal Fc receptor. Such compositions are useful in methods for the inhibition of viral growth and in methods for the treatment of viral infections, such as those caused by an influenza virus (e.g., influenza virus A, influenza virus B, or influenza virus C).

In an aspect, the disclosure features a method of synthesizing a conjugate of formula (D-I) or (M-I):

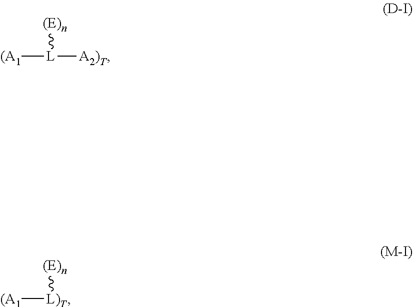

wherein A₁ and A₂ are each, independently, an anti-influenza moiety;
n is 1 or 2;
each E includes an Fc domain monomer (e.g., an Fc domain monomer having the sequence of anyone of SEQ ID NOs: 1-14) or an albumin protein;
L is a linker covalently attached to E and to A₁ (e.g., of M-I) or A₁ and A₂ (e.g., of D-I);
T is an integer from 1 to 20; and
each squiggly line in formula (M-I) or (D-I) indicates that L is covalently attached (e.g., by way of a covalent bond or linker) to each E,
the method including the steps of:
(a) providing a first composition including E;
(b) providing a second composition including a compound of formula (DF-I), (MF-I) or a salt thereof:

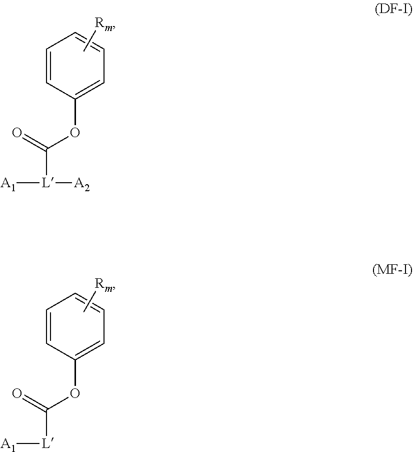

wherein
L′ is the remainder of L;
m is 0, 1, 2, 3, or 4; and
each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

In some embodiments, each anti-influenza moiety is a small molecule. In some embodiments, each anti-influenza moiety is selected from pimovidir, oseltamivir, zanamivir, sulfozanamivir, peramivir, Ianinamivir, amantadine, rimantadine, baloxavir (e.g., baloxavir acid or baloxavir marboxil), or an analog thereof. In some embodiments, each anti-influenza moiety is an inhibitor of viral neuraminidase, e.g., zanamivir, sulfozanamivir, peramivir, or an analog thereof.

In some embodiments, the compound is a conjugate of formula (D-I) and the second composition of (b) includes a compound of formula (DF-I) or a salt thereof.

In some embodiments, the compound is a conjugate of formula (M-I) and the second composition of (b) includes a compound of formula (MF-I) or a salt thereof.

In an aspect, the disclosure features a method of synthesizing a conjugate of formula (D-I) or (M-I):

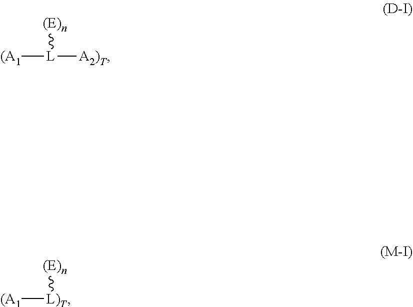

where each A₁ and each A₂ is, independently, selected from any one of formulas (A-I)-(A-XIII) (e.g., any one of formulas (A-I)-(A-VIII) or (A-IX)-(A-XIII)):

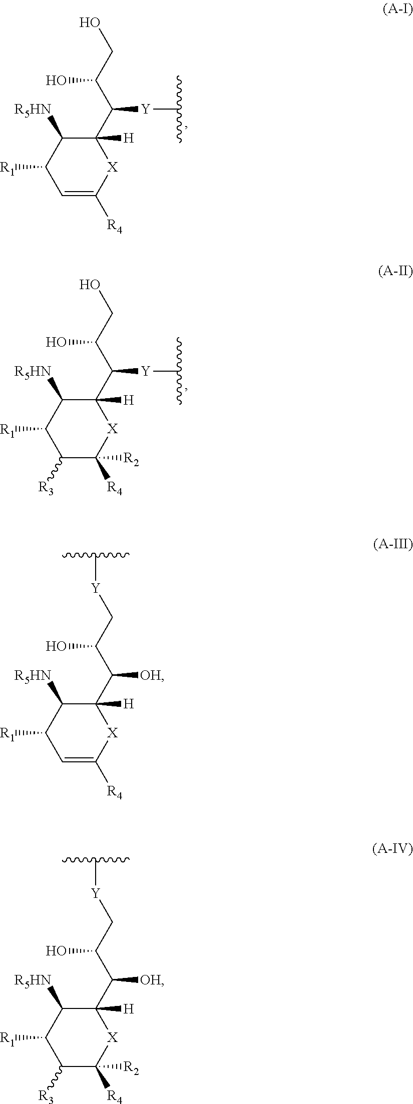

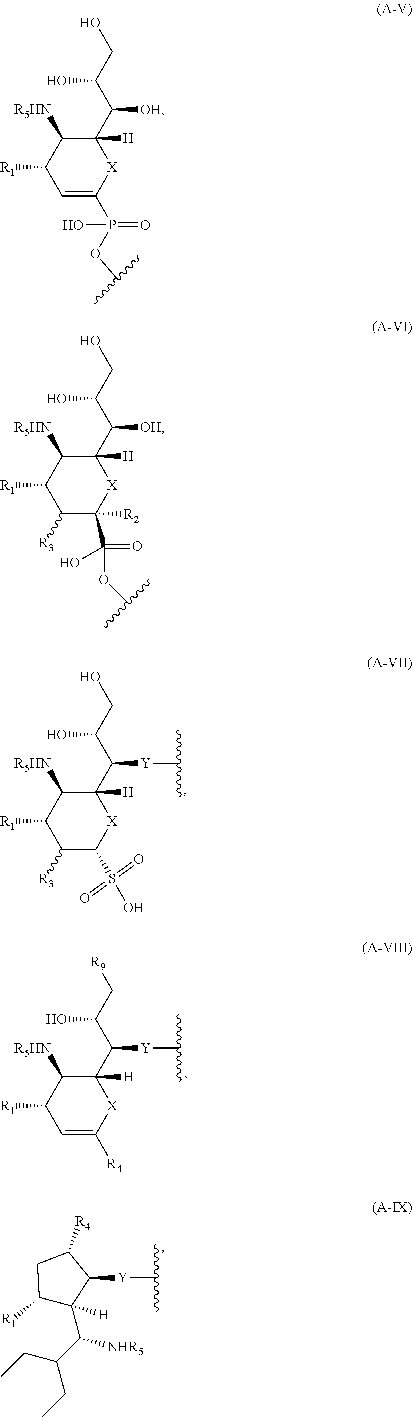

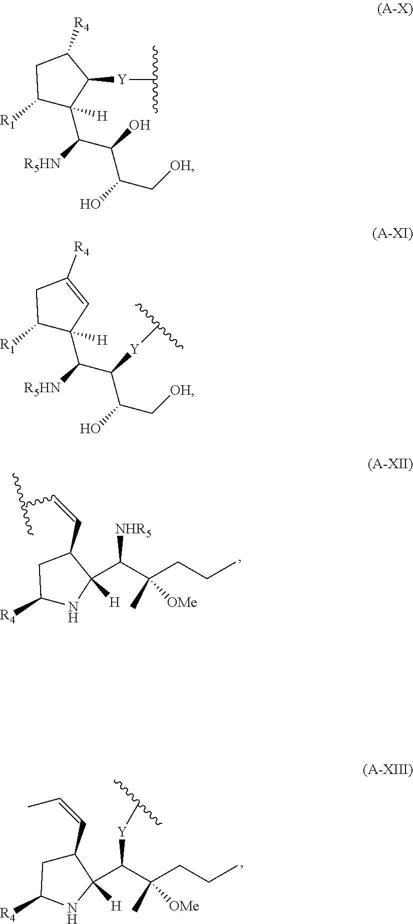

where R₁ is selected from —OH, —NH₂, —NHC(═NH)NH₂, and —NHC(═NH)NHR₆; R₂ and R₃ are each independently selected from —H, —OH, —F, —Cl, and —Br; R₄ is selected from —CO₂H, —P(═O)(OH)₂, —SO₃H; R₅ is selected from —COCH₃, —COCF₃, —SO₂CH₃; X is selected from —O— and —S—; Y is selected from

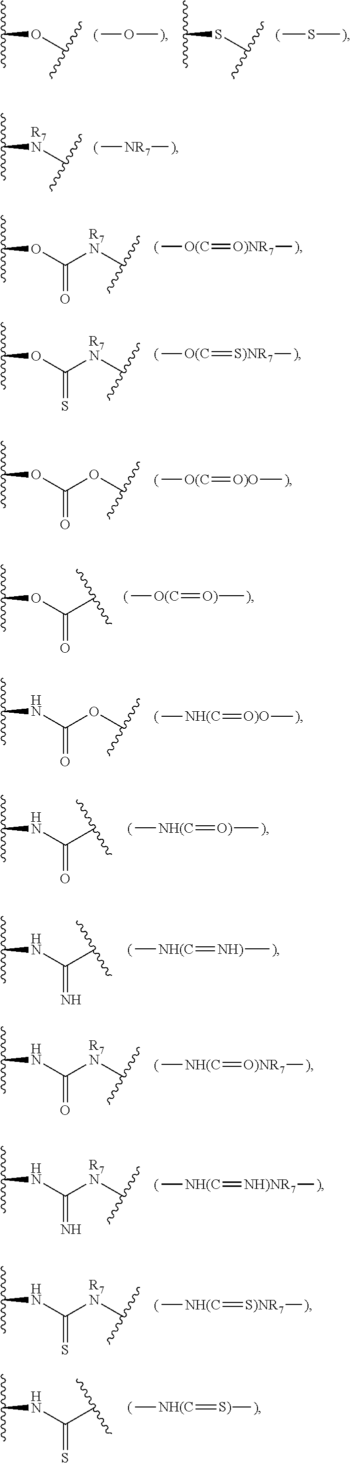

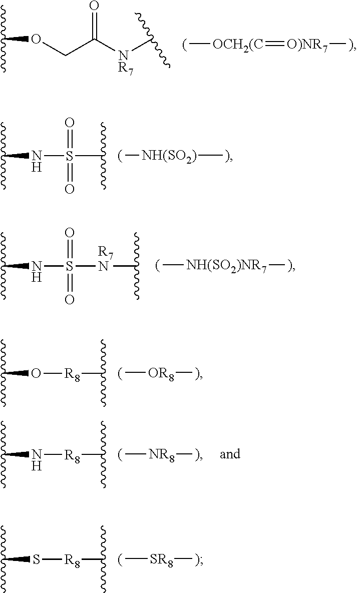

R₆ is selected from

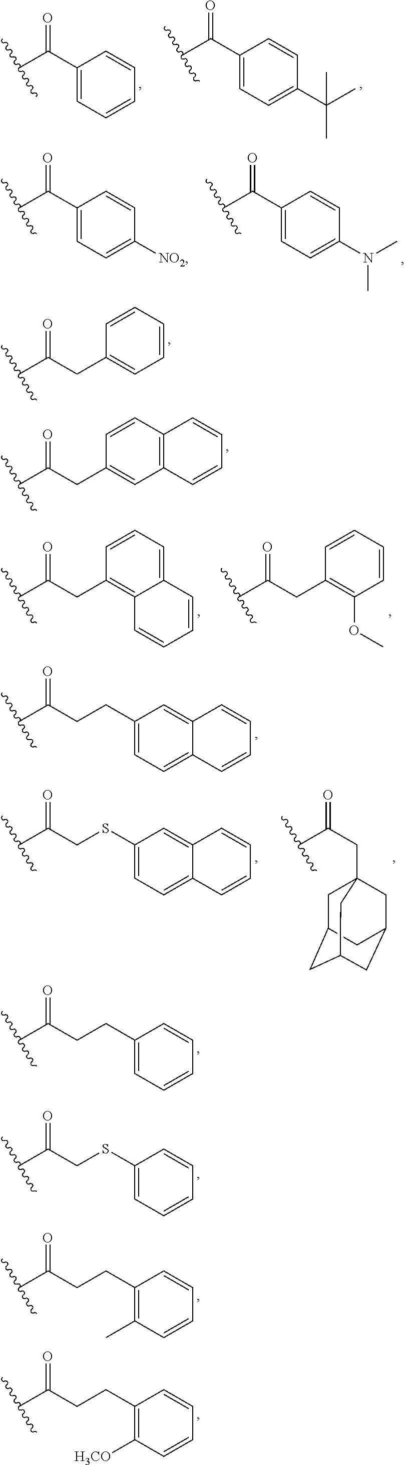

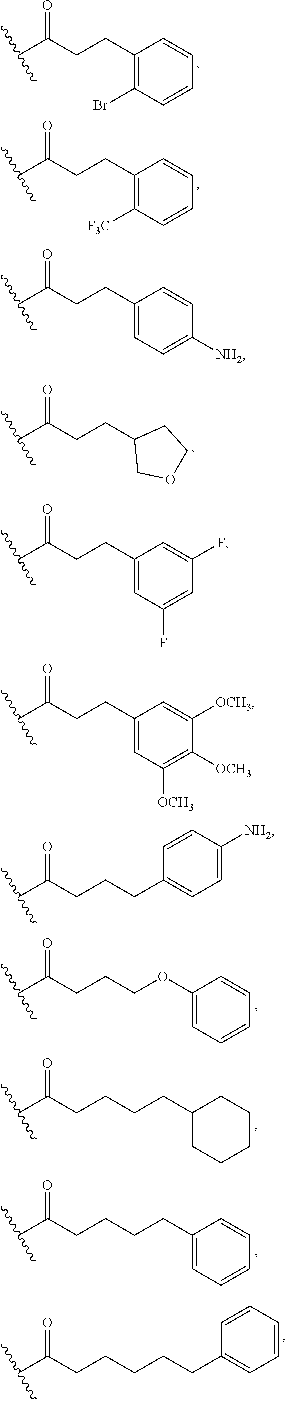

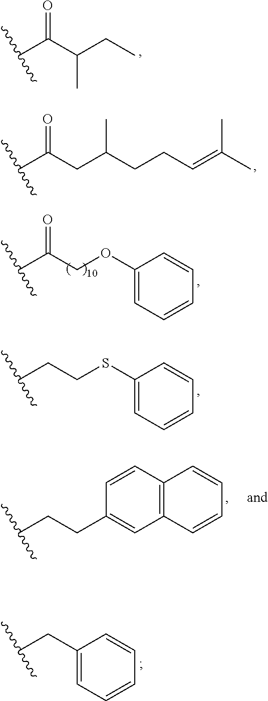

R₇ is selected from H, C1-C20 alkyl, C3-C20 cycloalkyl, C3-C20 heterocycloalkyl; C5-C15 aryl, and C2-C15 heteroaryl;
R₈ is selected from C3-C20 heterocycloalkyl, C5-C15 aryl, and C2-C15 heteroaryl;
R₉ is selected from —H, a halogen (e.g., Cl or F), —OR₁₀, —NHC(═O)R₇, optionally substituted C1-C20 alkyl, C3-C20 cycloalkyl, C3-C20 heterocycloalkyl; C5-C15 aryl, and C2-C15 heteroaryl; and
R₁₀ is selected from C1-C20 alkyl, C3-C20 cycloalkyl, C3-C20 heterocycloalkyl; C5-C15 aryl, and C2-C15 heteroaryl;
n is 1 or 2;
each E includes an Fc domain monomer (e.g., an Fc domain monomer having the sequence of anyone of SEQ ID NOs: 1-14) or an albumin protein;
L is a linker covalently attached to E and to Y of each of A₁ and A₂;
T is an integer from 1 to 20; and
each squiggly line in formula (D-I) or (M-I) indicates that L is covalently attached (e.g., by way of a covalent bond or linker) to each E,
the method including the steps of:
(a) providing a first composition including E;
(b) providing a second composition including a compound of formula (DF-I), (MF-I), or salt thereof:

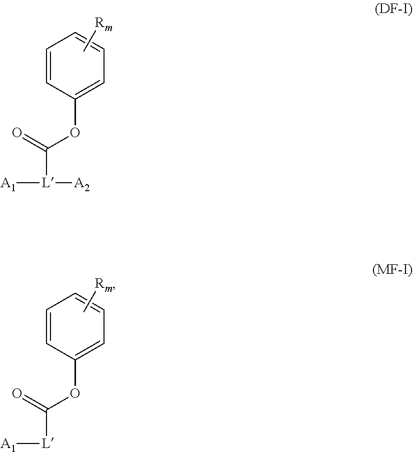

wherein
L′ is the remainder of L;
m is 0, 1, 2, 3, or 4; and
each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

In some embodiments, the first composition including E is an Fc domain (e.g., n is 2, each E is an Fc domain monomer, and the Fc domain monomers dimerize to form an Fc domain).

In some embodiments, L′ includes G, wherein G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ cycloalkylene, optionally substituted C₂-C₁₀ heterocycloalkylene, optionally substituted C₆-C₁₀ arylene, or optionally substituted C₂-C₁₀ heteroarylene.

In some embodiments, a compound of formula (DF-I) or salt thereof has the structure of any Int described herein (e.g., an Int of Table 1, for example, Int-93 or Int-94). In some embodiments, a compound of formula (DF-I) or salt thereof is synthesized from the structure of any Int described herein (e.g., an Int of Table 1, for example, Int-93 or Int-94).

In some embodiments, a compound of formula (MF-I) or salt thereof has the structure of any Int described in WO 2020/051498 and WO 2021/046549, each of which is hereby incorporated by reference.

In some embodiments, a compound of formula (MF-I) or salt thereof is synthesized from the structure of any Int described in WO 2020/051498 and WO 2021/046549, each of which is hereby incorporated by reference.

In some embodiments, a compound of formula (DF-I) or (MF-I), where each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-I) or (MF-I) where m is 3, provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-I) or (MF-I) where m is 3 and each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In an aspect, the disclosure features a method of synthesizing a conjugate of formula (D-I) or (M-I):

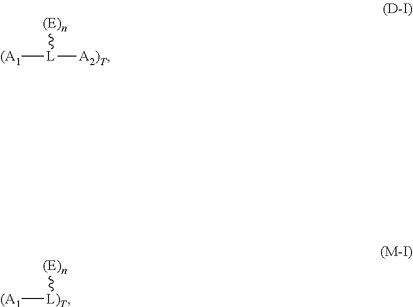

wherein each A₁ and each A₂ is independently selected from any one of formulas (A-I)-(A-XIII) (e.g., any one of formulas (A-I)-(A-VIII) or (A-IX)-(A-XIII)):

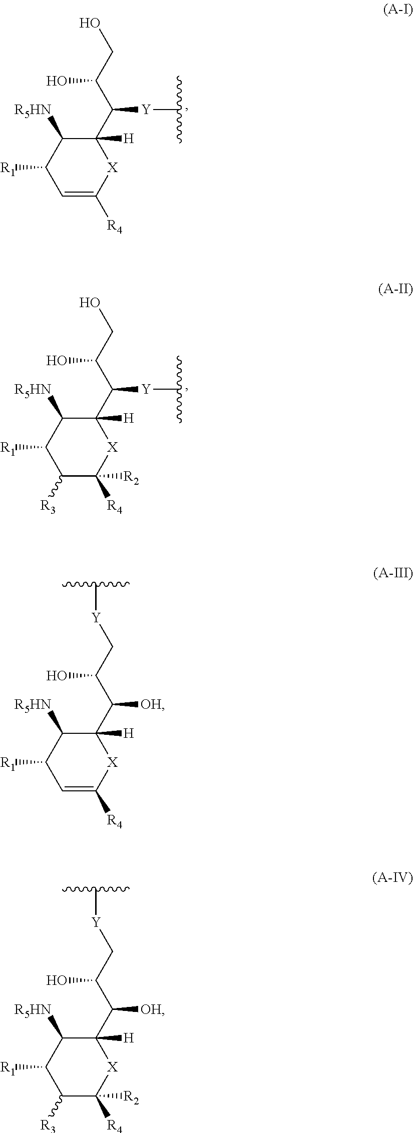

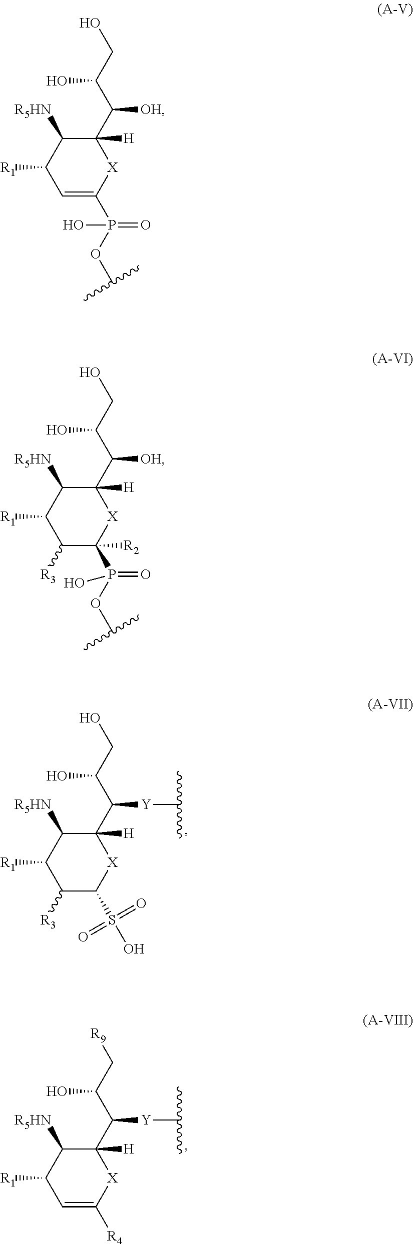

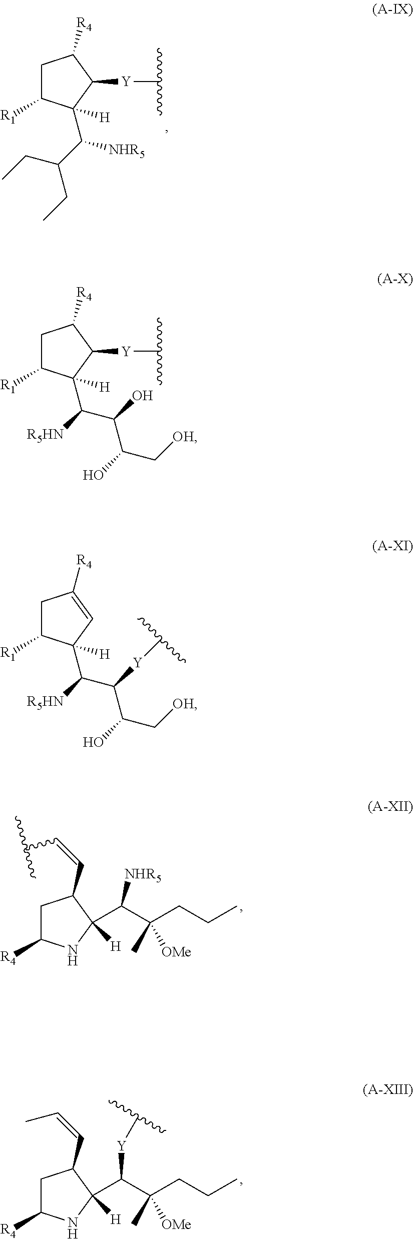

where R₁ is selected from —OH, —NH₂, —NHC(═NH)NH₂, and —NHC(═NH)NHR₆; R₂ and R₃ are each independently selected from —H, —OH, —F, —Cl, and —Br; R₄ is selected from —CO₂H, —P(═O)(OH)₂, —SO₃H; R₅ is selected from —COCH₃, —COCF₃, —SO₂CH₃; X is selected from —O— and —S—; Y is selected from

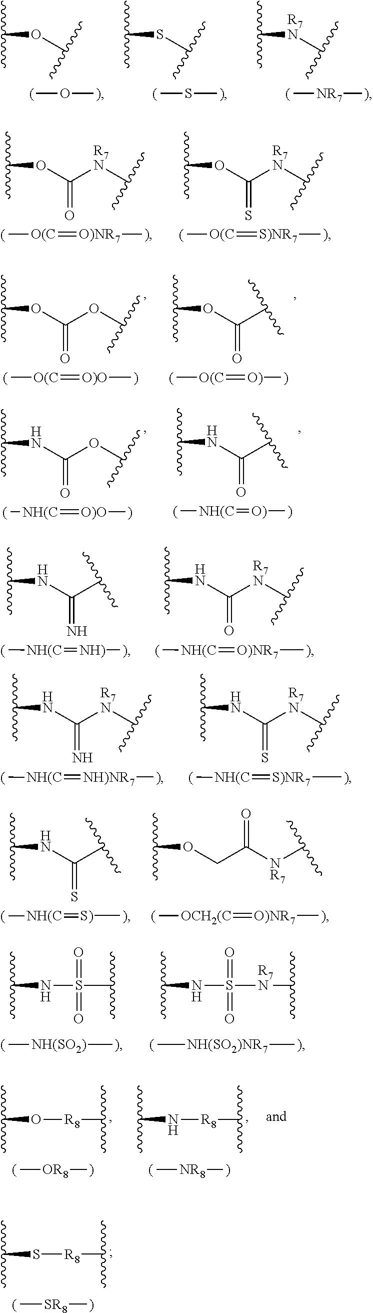

R₆ is selected from

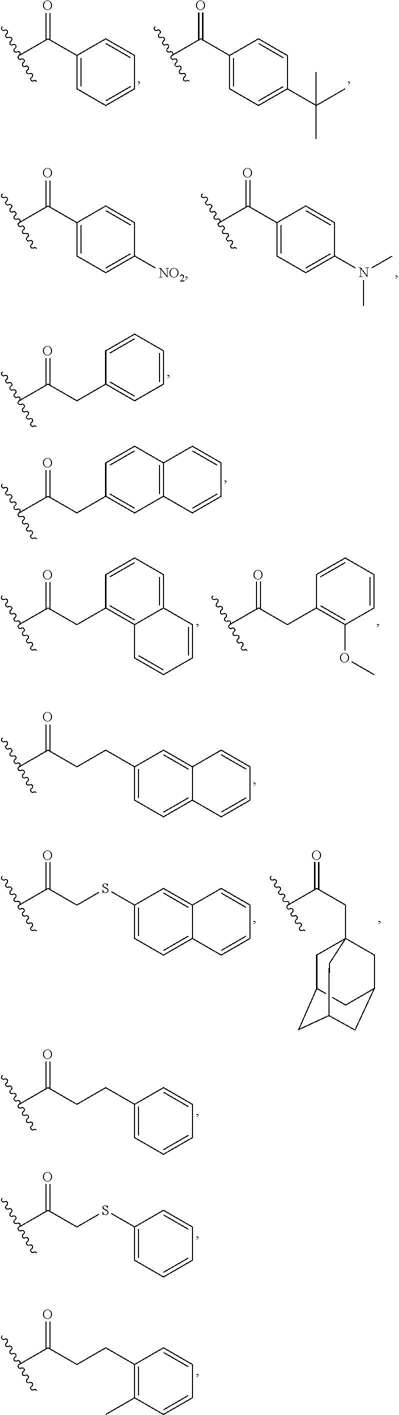

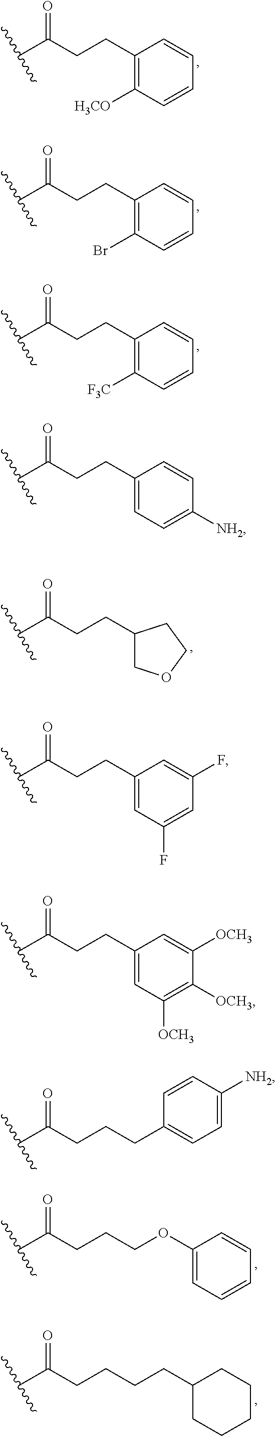

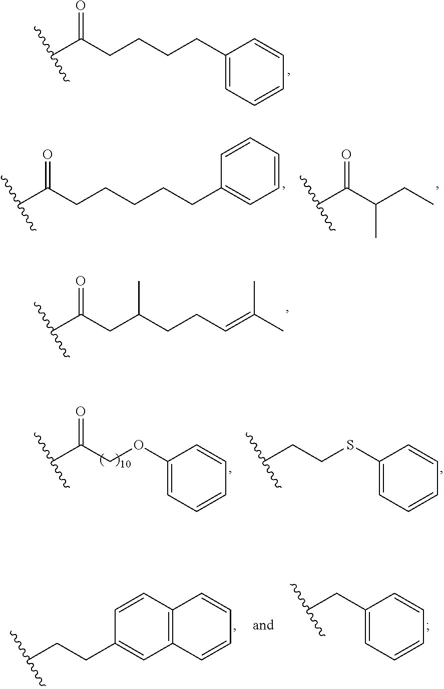

R₇ is selected from H, C1-C20 alkyl, C3-C20 cycloalkyl, C3-C20 heterocycloalkyl; C5-C15 aryl, and C2-C15 heteroaryl;
R₈ is selected from C3-C20 heterocycloalkyl, C5-C15 aryl, and C2-C15 heteroaryl;
R₉ is selected from —H, a halogen (e.g., Cl or F), —OR₁₀, —NHC(═O)R₇, optionally substituted C1-C20 alkyl, C3-C20 cycloalkyl, C3-C20 heterocycloalkyl; C5-C15 aryl, and C2-C15 heteroaryl; and
R₁₀ is selected from C1-C20 alkyl, C3-C20 cycloalkyl, C3-C20 heterocycloalkyl; C5-C15 aryl, and C2-C15 heteroaryl;
n is 1 or 2;
each E includes an Fc domain monomer (e.g., an Fc domain monomer having the sequence of anyone of SEQ ID NOs: 1-14) or an albumin protein;
L is a linker covalently attached to E and to Y of each of A₁ and A₂;
T is an integer from 1 to 20; and
each squiggly line in formula (D-I) or (M-I) indicates that L is covalently attached (e.g., by way of a covalent bond or linker) to each E,
the method including the steps of:
(a) providing a first composition including E;
(b) providing a second composition including a compound of formula (DF-II), (MF-II), or a salt thereof:

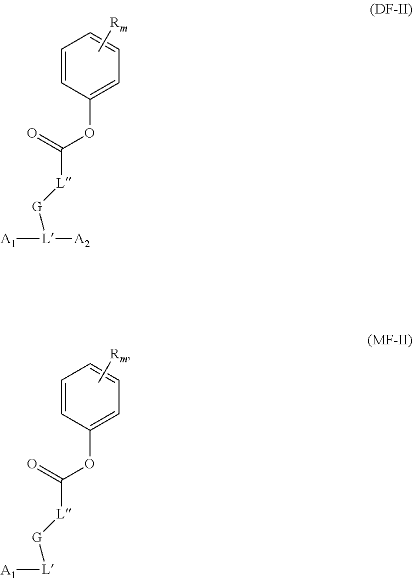

wherein G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ cycloalkylene, optionally substituted C₂-C₁₀ heterocycloalkylene, optionally substituted C₆-C₁₀ arylene, or optionally substituted C₂-C₁₀ heteroarylene;
L′-G-L″ is the remainder of L;
m is 0, 1, 2, 3, or 4; and
each R is, independently, halo, cyano, nitro, optionally substituted C₁-C₆ alkyl group, or optionally substituted C₁-C₆ heteroalkyl group;
and
(c) combining the first composition, the second composition, and a buffer to form a mixture.

In some embodiments, the first composition including E is an Fc domain (e.g., n is 2, each E is an Fc domain monomer, and the Fc domain monomers dimerize to form an Fc domain.

In some embodiments, G is optionally substituted C₁-C₆ heteroalkylene or optionally substituted C₂-C₁₀ heteroarylene. In some embodiments, G is optionally substituted C₁-C₆ heteroalkylene.

In some embodiments, G is

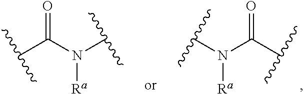

where R^{a} is H, optionally substituted C₁-C₂₀alkylene (e.g., optionally substituted C₁-C₆ alkylene), or optionally substituted C₁-C₂₀ heteroalkylene (e.g., optionally substituted C₁-C₆ heteroalkylene).

In some embodiments, G is optionally substituted C₂-C₁₀ heteroarylene. In some embodiments, G is optionally substituted C₂-C₅ heteroarylene. In some embodiments, G is a 5-membered or 6-membered optionally substituted C₂-C₅ heteroarylene. In some embodiments, G is a triazolylene.

In some embodiments, the conjugate of formula (D-I) has the structure of:

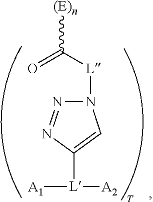

and the method includes the steps of:
(a) providing a first composition including E;
(b) providing a second composition including a compound of formula (DF-II-A) or salt thereof:

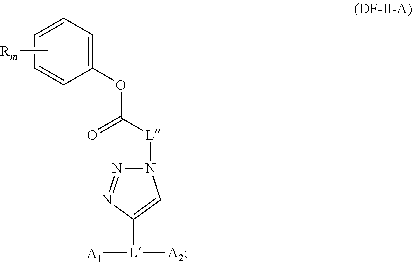

and
(c) combining the first composition, the second composition, and a buffer to form a mixture.

In some embodiments, the synthesis of compound of formula (DF-II-A) includes:
(d) providing a third composition including formula (D-G1-A) or salt thereof:

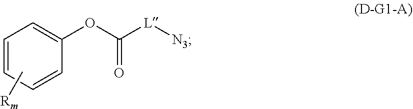

(e) providing a fourth composition including formula (D-G1-B) or salt thereof:

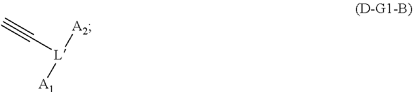

and
(f) combining the third composition and the fourth composition to form a mixture.

In some embodiments, the conjugate of formula (D-I) has the structure of:

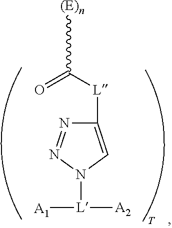

and the method includes the steps of:
(a) providing a first composition including E;
(b) providing a second composition including a compound of formula (DF-II-B) or salt thereof:

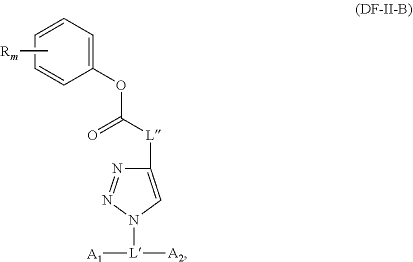

and
(c) combining the first composition, the second composition, and a buffer to form a mixture.

In some embodiments, the synthesis of compound of formula (DF-II-B) includes:
(d) providing a third composition including formula (D-G2-A) or salt thereof:

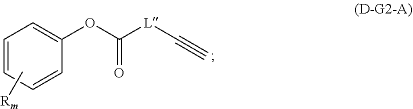

(e) providing a fourth composition including formula (D-G2-B) or salt thereof:

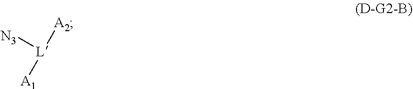

and
(f) combining the third composition and the fourth composition to form a mixture.

In some embodiments, step (f) includes the use of a Cu(I) source.

In some embodiments, the compound of formula (D-FI) or (DF-II) has the structure:

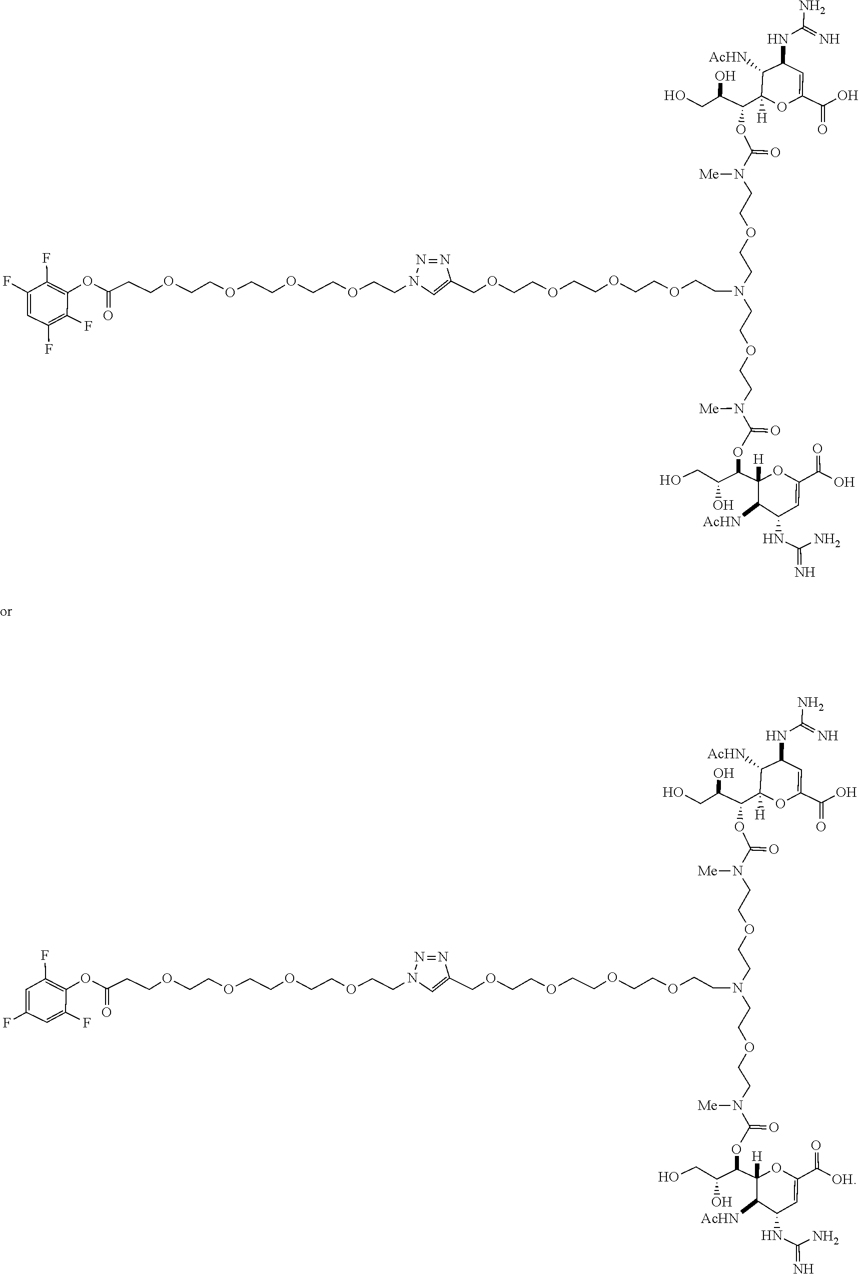 [SMILES: CC(=O)N[C@H]1[C@H]([C@H](OC(=O)N(C)CCOCCN(CCOCCOCCOCCOCc2cn(CCOCCOCCOCCOCCC(=O)Oc3c(F)c(F)cc(F)c3F)nn2)CCOCCN(C)C(=O)O[C@@H]([C@@H]2OC(C(=O)O)=C[C@H](NC(=N)N)[C@H]2NC(C)=O)[C@H](O)CO)[C@H](O)CO)OC(C(=O)O)=C[C@@H]1NC(=N)N ; CC(=O)N[C@H]1[C@H]([C@H](OC(=O)N(C)CCOCCN(CCOCCOCCOCCOCc2cn(CCOCCOCCOCCOCCC(=O)Oc3c(F)cc(F)cc3F)nn2)CCOCCN(C)C(=O)O[C@@H]([C@@H]2OC(C(=O)O)=C[C@H](NC(=N)N)[C@H]2NC(C)=O)[C@H](O)CO)[C@H](O)CO)OC(C(=O)O)=C[C@@H]1NC(=N)N]

In some embodiments, the conjugate of formula (M-I) has the structure of:

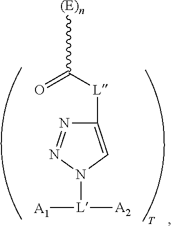

and the method including the steps of:
(a) providing a first composition including E;
(b) providing a second composition including a compound of formula (MF-II-A) or salt thereof:

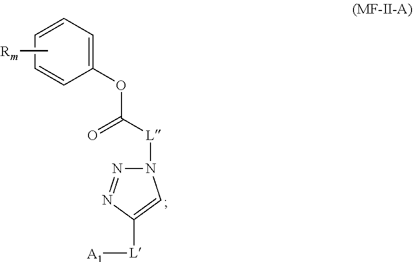

and
(c) combining the first composition, the second composition, and a buffer to form a mixture.

In some embodiments, the synthesis of compound of formula (MF-II-A) includes:
(d) providing a third composition including formula (M-G1-A) or salt thereof:

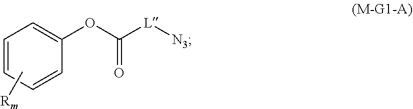

(e) providing a fourth composition including formula (M-G1-B) or salt thereof:

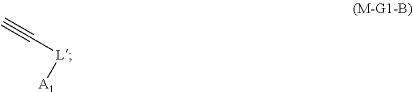

and
(f) combining the third composition and the fourth composition to form a mixture.

In some embodiments, the conjugate of formula (M-I) has the structure of:

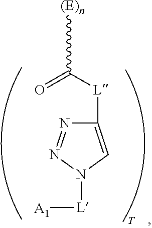

and the method includes the steps of:
(a) providing a first composition including E;
(b) providing a second composition including a compound of formula (MF-II-B) or salt thereof:

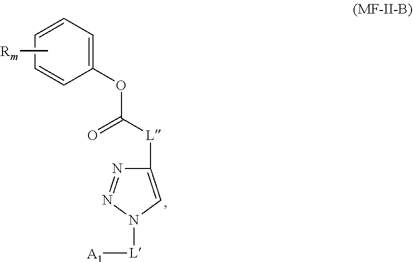

and
(c) combining the first composition, the second composition, and a buffer to form a mixture.

In some embodiments, the synthesis of compound of formula (MF-II-B) includes:
(d) providing a third composition including formula (M-G2-A) or salt thereof:

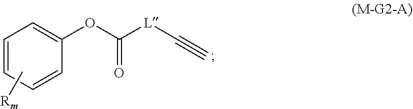

(e) providing a fourth composition including formula (M-G2-B) or salt thereof:

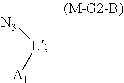

and
(f) combining the third composition and the fourth composition to form a mixture.

In some embodiments, step (f) includes the use of a Cu(I) source.

In an aspect, the disclosure features a method of synthesizing a conjugate of formula (D-I) or (M-I):

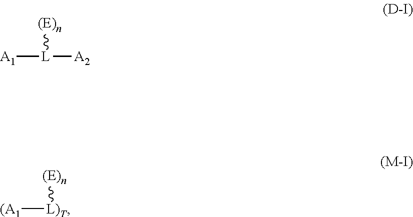

wherein each A₁ and each A₂ is independently selected from an anti-influenza moiety or any one of formulas (A-I)-(A-XIII) (e.g., any one of formulas (A-I)-(A-VIII) or (A-IX)-(A-XIII)):

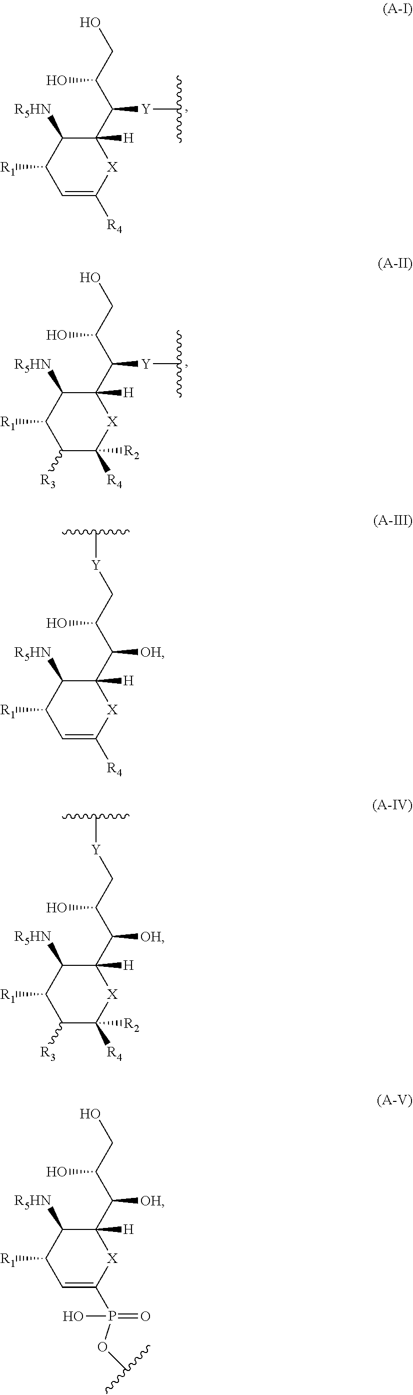

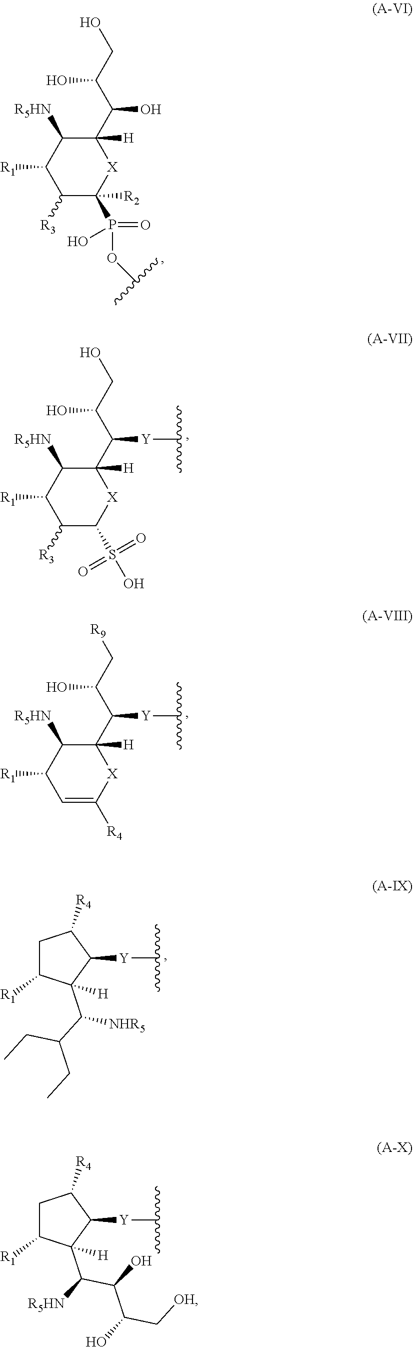

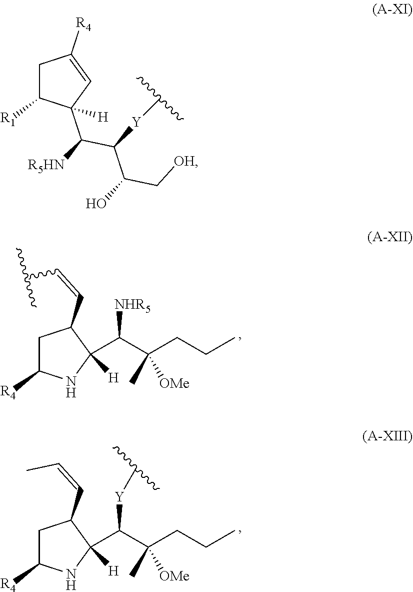

where R₁ is selected from —OH, —NH₂, —NHC(═NH)NH₂, and —NHC(═NH)NHR₆; R₂ and R₃ are each independently selected from —H, —OH, —F, —Cl, and —Br; R₄ is selected from —CO₂H, —P(═O)(OH)₂, —SO₃H; R₅ is selected from —COCH₃, —COCF₃, —SO₂CH₃; X is selected from —O— and —S—; Y is selected from

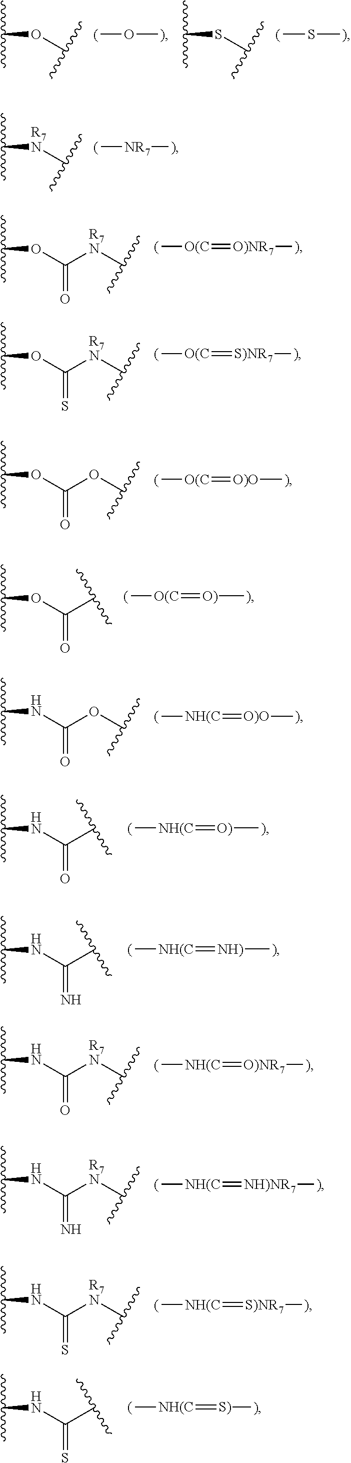

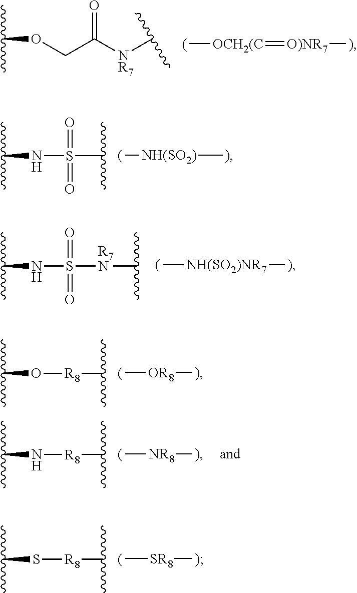

R₆ is selected from

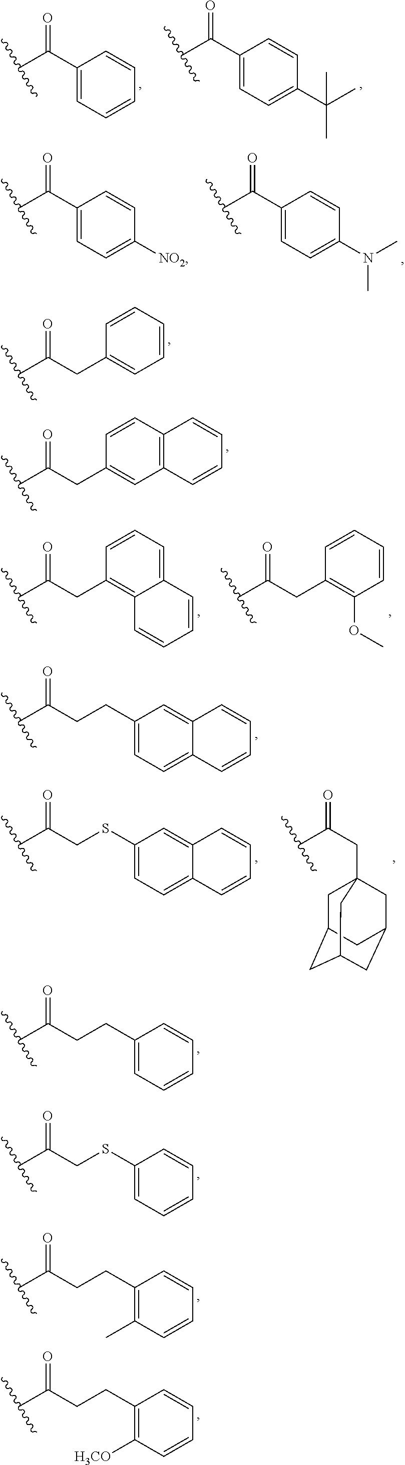

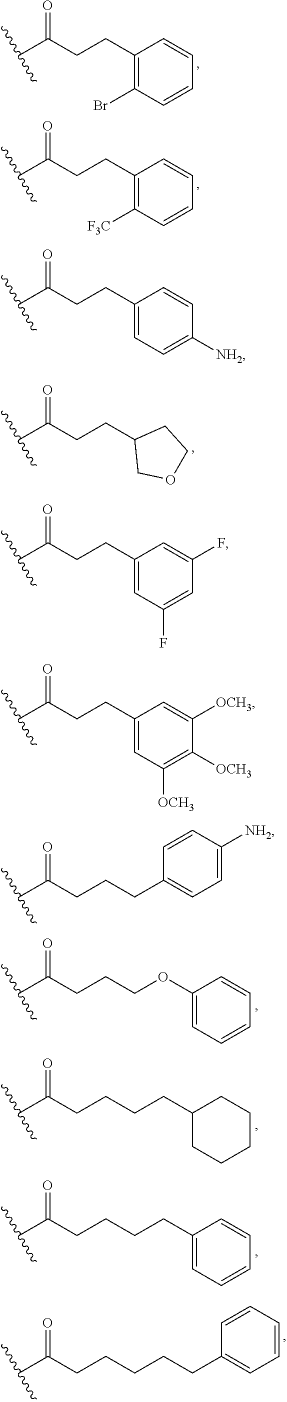

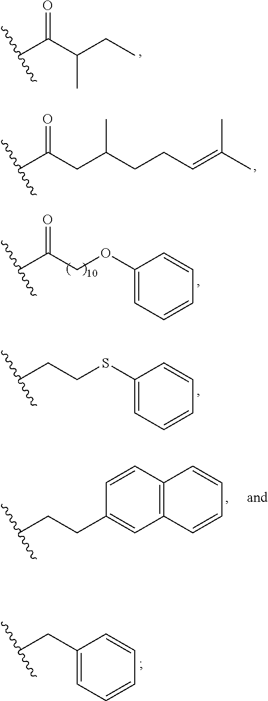

R₇ is selected from H, C1-C20 alkyl, C3-C20 cycloalkyl, C3-C20 heterocycloalkyl; C5-C15 aryl, and C2-C15 heteroaryl;
R₈ is selected from C3-C20 heterocycloalkyl, C5-C15 aryl, and C2-C15 heteroaryl;
R₉ is selected from —H, a halogen (e.g., Cl or F), —OR₁₀, —NHC(═O)R₇, optionally substituted C1-C20 alkyl, C3-C20 cycloalkyl, C3-C20 heterocycloalkyl; C5-C15 aryl, and C2-C15 heteroaryl; and
R₁₀ is selected from C1-C20 alkyl, C3-C20 cycloalkyl, C3-C20 heterocycloalkyl; C5-C15 aryl, and C2-C15 heteroaryl;
n is 1 or 2;
each E includes an Fc domain monomer (e.g., an Fc domain monomer having the sequence of anyone of SEQ ID NOs: 1-14) or an albumin protein;
L is a linker covalently attached to E and to Y of each of A₁ and A₂;
T is an integer from 1 to 20; and
each squiggly line in formula (D-I) or (M-I) indicates that L is covalently attached (e.g., by way of a covalent bond or linker) to each E,
the method including the steps of:
(a) providing a first composition including formula (D-G3-A) or (M-G3-A) or a salt thereof:

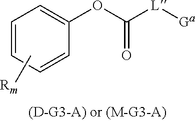

where G^{a} is a functional group that reacts with G^{b} to form G;
(b) providing a second composition including formula (D-G3-B), (M-G3-B), or a salt thereof:

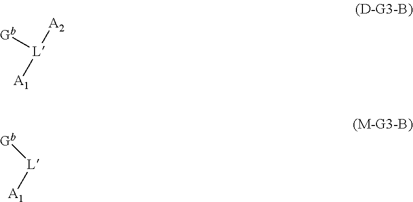

where G^{b} is a functional group that reacts with G^{a} to form G; and
(c) combining the first composition and the second composition to form a first mixture,
where m is 0, 1, 2, 3, or 4; and each R is, independently, halo, cyano, nitro, optionally substituted C₁-C₆ alkyl group, or optionally substituted C₁-C₆ heteroalkyl group.

In some embodiments, step (c) includes the use of a Cu(I) source.

In some embodiments, the method further includes:
(d) providing a third composition including E; and
(e) combining the third composition, the first mixture, and a buffer to form a second mixture.

In some embodiments, G^{a} includes optionally substituted amino. In some embodiments, G^{b} includes a carbonyl.

In some embodiments, G^{a} includes a carbonyl. In some embodiments, G^{b} includes optionally substituted amino.

In some embodiments, G^{a} includes an azido group. In some embodiments, G^{b} includes an alknyl group.

In some embodiments, G^{a} includes an alkynyl group. In some embodiments, G^{b} includes an azido group.

In some embodiments of any of the aspects described herein, a compound of formula (DF-II) or salt thereof has the structure of any Int described herein (e.g., an Int of Table 1, for example, Int-93 or Int-94). In some embodiments of any of the aspects described herein, a compound of formula (DF-II) or salt thereof is synthesized from the structure of any Int described herein (e.g., an Int of Table 1, for example, Int-93 or Int-94).

In some embodiments of any of the aspects described herein, a compound of formula (MF-II) or salt thereof has the structure of any Int described by WO 2020/051498 and WO 2021/046549, each of which is hereby incorporated by reference. In some embodiments of any of the aspects described herein, a compound of formula (MF-II) or salt thereof is synthesized from the structure of any Int described by WO 2020/051498 and WO 2021/046549, each of which is hereby incorporated by reference

In some embodiments, a compound of formula (DF-II) or (MF-II) (e.g., a compound of formula (DF-II-A), (DF-II-B), (MF-II-A), or (MF-II-B) and/or a compound of formula (D-G1-A), (D-G2-A), (D-G1-A), or (D-G2-A)), where each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-II) or (MF-II) (e.g., a compound of formula (DF-II-A), (DF-II-B), (MF-II-A), or (MF-II-B) and/or a compound of formula (D-G1-A), (D-G2-A), (M-G1-A), or (M-G2-A)), where m is 3, provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments, a compound of formula (DF-II) or MF-II (e.g., a compound of formula (DF-II-A), (DF-II-B), (MF-II-A), or (MF-II-B) and/or a compound of formula (D-G1-A), (D-G2-A), (MF-II-A), or (MF-II-B)), where m is 3 and each R is halo (e.g., F), provides technical advantages (e.g., increased stability) in methods of synthesizing protein-drug conjugates (e.g., the methods described herein). In some embodiments, the increased stability allows for purification by reverse phase chromatography. In some embodiments, the increased stability allows for lyophilization with minimal hydrolysis of the activated ester.

In some embodiments of any of the aspects described herein, E includes at least one lysine residue. In some embodiments, the squiggly line in formula (D-I) or (M-I) is covalently bound to a lysine residue of each E.

In some embodiments of any of the aspects described herein, E includes at least one cysteine residue. In some embodiments, the squiggly line in formula (D-I) or (M-I) is covalently bound to a cysteine residue of each E.

In some embodiments of any of the aspects described herein, each R is, independently, halo, cyano, nitro, haloalkyl, or

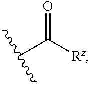

where R^{z} is optionally substituted C₁-C₅ alkyl group or optionally substituted C₁-C₅ heteroalkyl group. In some embodiments, each R is, independently, halo, cyano, nitro, or haloalkyl.

In some embodiments, each R is, independently, F, Cl, Br, or I.

In some embodiments, each R is F.

In some embodiments of any of the aspects described herein, m is 1, 2, 3, or 4. In some embodiments, m is 3 or 4. In some embodiments, m is 3. In some embodiments, m is 4.

In some embodiments,

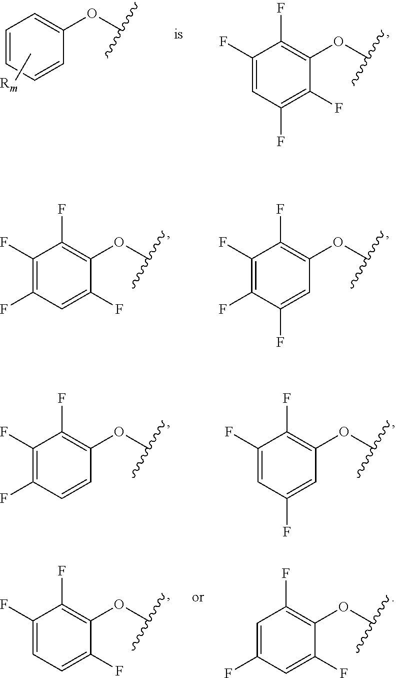

In some embodiments,

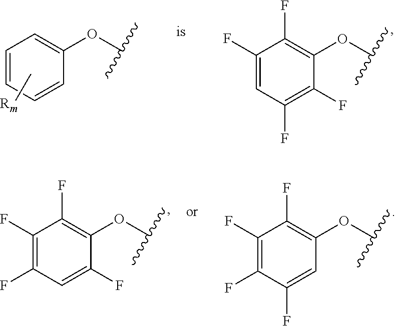

In some embodiments,

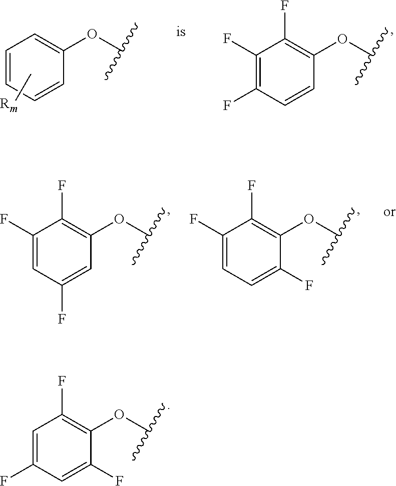

In some embodiments,

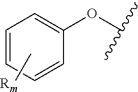

is

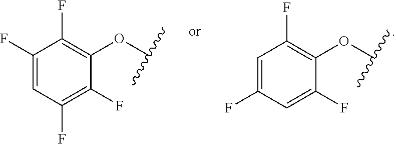 [SMILES: *Oc1c(F)c(F)cc(F)c1F ; *Oc1c(F)cc(F)cc1F]

In some embodiments,

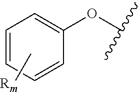

is

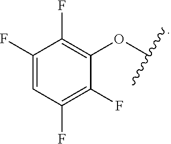 [SMILES: *Oc1c(F)c(F)cc(F)c1F]

In some embodiments,

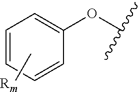

is

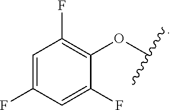 [SMILES: *Oc1c(F)cc(F)cc1F]

In some embodiments of any of the aspects described herein, the buffer includes borate or carbonate. In some embodiments, the buffer includes borate. In some embodiments, the buffer includes carbonate.

In some embodiments, the buffer has a pH of about 7.0 to 10.0 (e.g., about 7.0 to 7.5, 7.5 to 8.0, 8.0 to 8.5, 8.5 to 9.0, 9.0 to 9.5, 9.5 to 10.0, 7.0 to 8.0, 7.5 to 8.5, 8.0 to 9.0, 8.5 to 9.5, 9.0 to 10.0, 7.0 to 9.0, 7.5 to 9.5, or 8.0 to 10.0).

In some embodiments, the buffer has a pH of about 7.0. In some embodiments, the buffer has a pH of about 7.1. In some embodiments, the buffer has a pH of about 7.2. In some embodiments, the buffer has a pH of about 7.3. In some embodiments, the buffer has a pH of about 7.4. In some embodiments, the buffer has a pH of about 7.5. In some embodiments, the buffer has a pH of about 7.6. In some embodiments, the buffer has a pH of about 7.7. In some embodiments, the buffer has a pH of about 7.8. In some embodiments, the buffer has a pH of about 7.9. In some embodiments, the buffer has a pH of about 8.0. In some embodiments, the buffer has a pH of about 8.1. In some embodiments, the buffer has a pH of about 8.2. In some embodiments, the buffer has a pH of about 8.3. In some embodiments, the buffer has a pH of about 8.4. In some embodiments, the buffer has a pH of about 8.5. In some embodiments, the buffer has a pH of about 8.6. In some embodiments, the buffer has a pH of about 8.7. In some embodiments, the buffer has a pH of about 8.8. In some embodiments, the buffer has a pH of about 8.9. In some embodiments, the buffer has a pH of about 9.0. In some embodiments, the buffer has a pH of about 9.5. In some embodiments, the buffer has a pH of about 9.6. In some embodiments, the buffer has a pH of about 9.7. In some embodiments, the buffer has a pH of about 9.8. In some embodiments, the buffer has a pH of about 9.9. In some embodiments, the buffer has a pH of about 10.0.

In some embodiments of any of the aspects described herein, step (c) or step (e) is conducted at a temperature of 5 to 50° C., such as 20 to 30° C. (e.g., 20 to 25, 21 to 26, 22 to 27, 23 to 28, 24 to 29, or 25 to 30° C.).

In some embodiments, step (c) or step (e) is conducted at a temperature of about 25° C.

In some embodiments, step (c) or step (e) is conducted for about 1 to 24 hours, such as 1 to 12 hours (e.g., 1 to 2, 1 to 5, 2 to 3, 2 to 5, 2 to 10, 2 to 12, 3 to 4, 4 to 5, 1 to 3, 2 to 4, or 3 to 5 hours).

In some embodiments, step (c) or step (e) is conducted for about 2 hours. In some embodiments, step (c) or step (e) is conducted for about 3 hours. In some embodiments, step (c) or step (e) is conducted for about 4 hours. In some embodiments, step (c) or step (e) is conducted for about 5 hours. In some embodiments, step (c) or step (e) is conducted for about 6 hours. In some embodiments, step (c) or step (e) is conducted for about 7 hours. In some embodiments, step (c) or step (e) is conducted for about 8 hours. In some embodiments, step (c) or step (e) is conducted for about 9 hours. In some embodiments, step (c) or step (e) is conducted for about 10 hours. In some embodiments, step (c) or step (e) is conducted for about 11 hours. In some embodiments, step (c) or step (e) is conducted for about 12 hours.

In some embodiments, the first composition or third composition includes phosphate-buffered saline buffer.

In some embodiments, the buffer has a pH of about 7.0 to 8.0 (e.g., about 7.0 to 7.5, 7.5 to 8.0, 7.0 to 7.2, 7.2 to 7.4, 7.4 to 7.6, 7.6 to 7.8, or 7.8 to 8.0).

In some embodiments, the buffer has a pH of about 7.5.

In some embodiments, the second composition or the first mixture includes DMF.

In some embodiments, the method further includes a purification step. In some embodiments, the purification step includes dialysis in arginine buffer. In some embodiments, the purification step includes a buffer exchange.

In some embodiments of any of the aspects described herein, the conjugate of formula (D-I) has the structure:

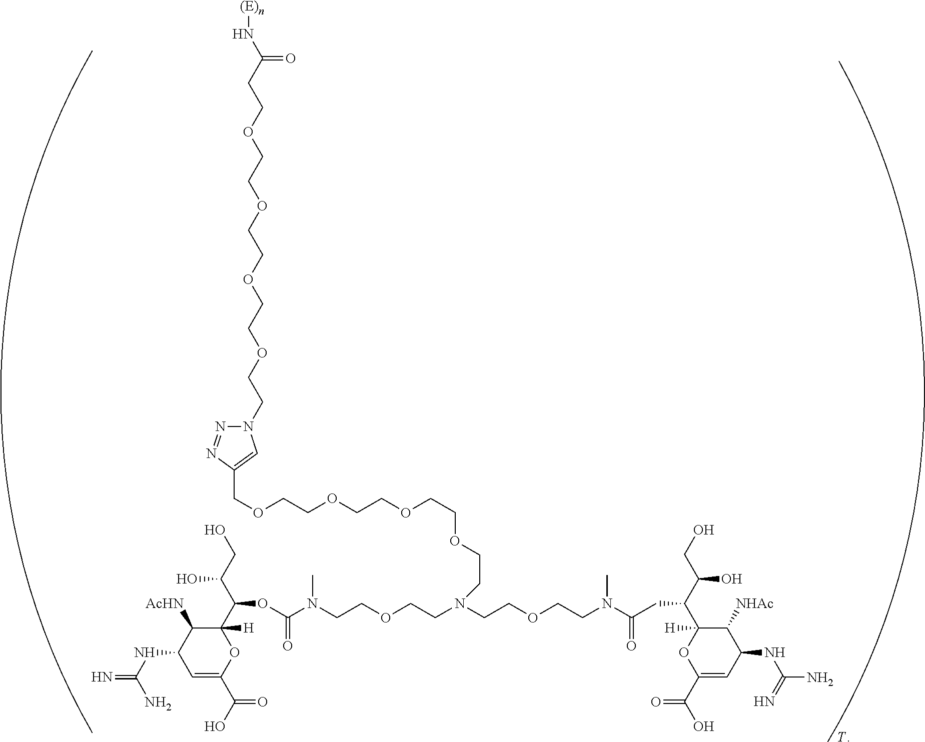

In some embodiments, the conjugate is described by formula (D-II):

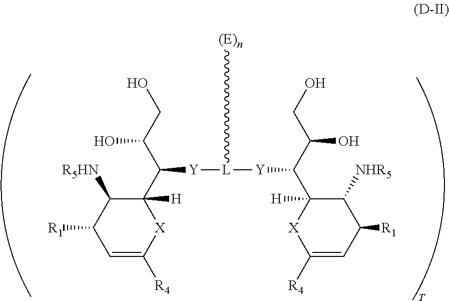

or a pharmaceutically acceptable salt thereof.

In some embodiments, the conjugate is described by formula (D-II-1):

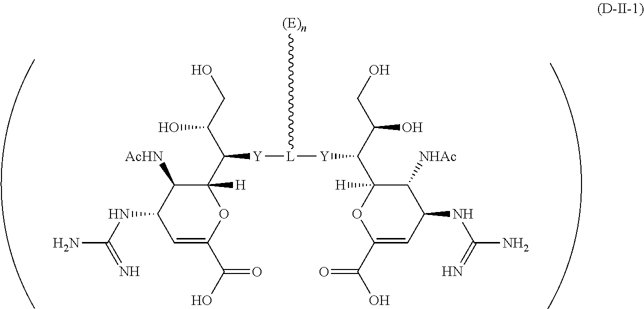

or a pharmaceutically acceptable salt thereof.

In some embodiments, the conjugate is described by formula (D-II-2):

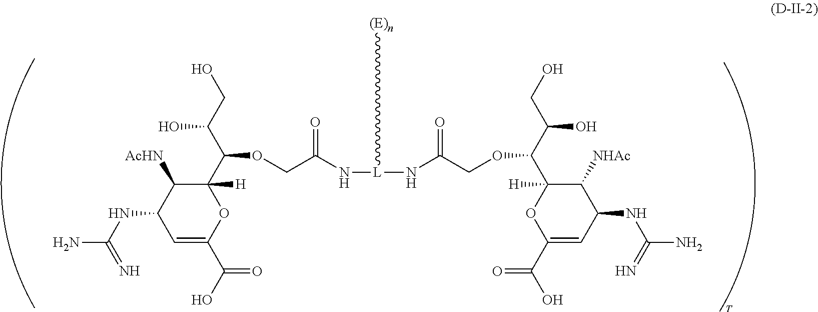

or a pharmaceutically acceptable salt thereof.

In some embodiments, the conjugate is described by formula (D-II-3):

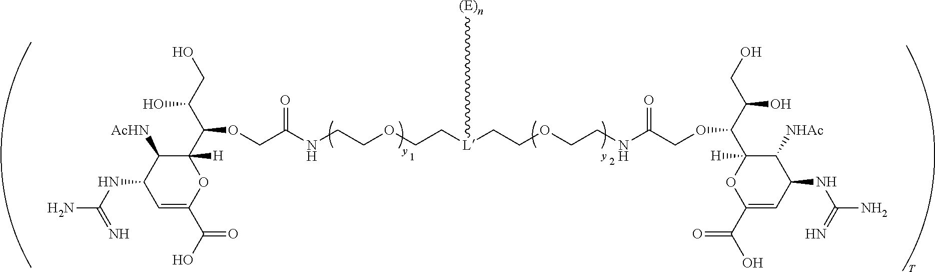

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure selected from:

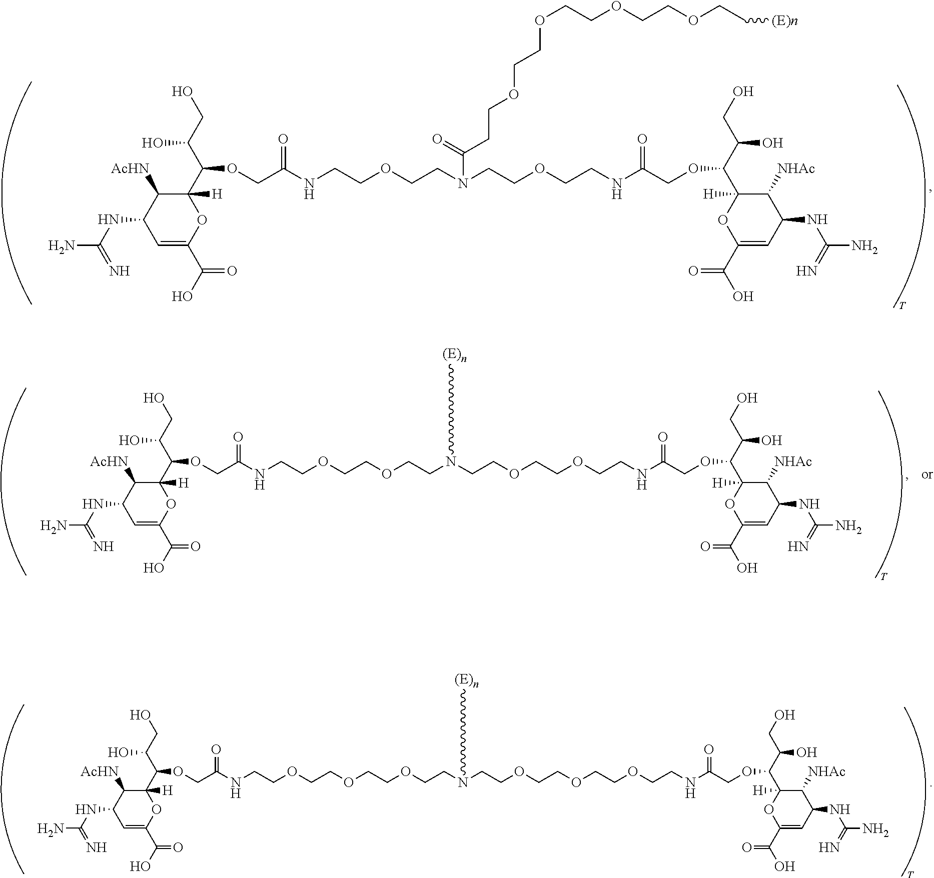

In some embodiments, the conjugate is described by formula (D-II-4):

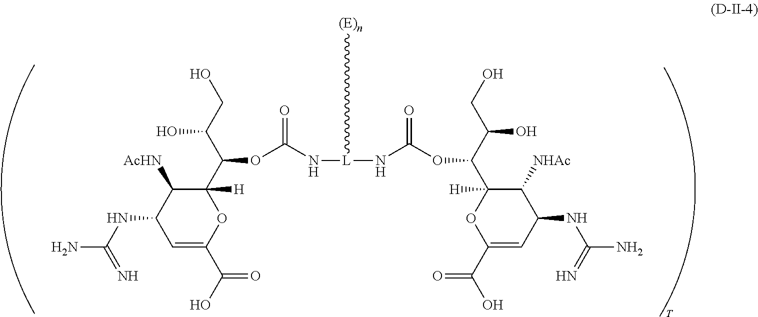

or a pharmaceutically acceptable salt thereof.

In some embodiments, the conjugate is described by formula (D-II-5):

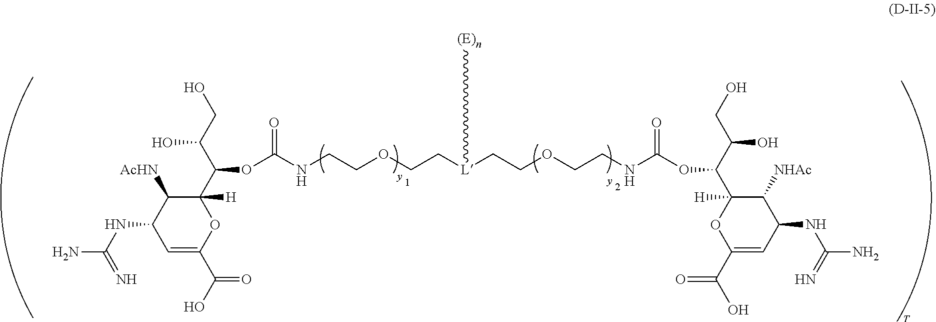

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure selected from:

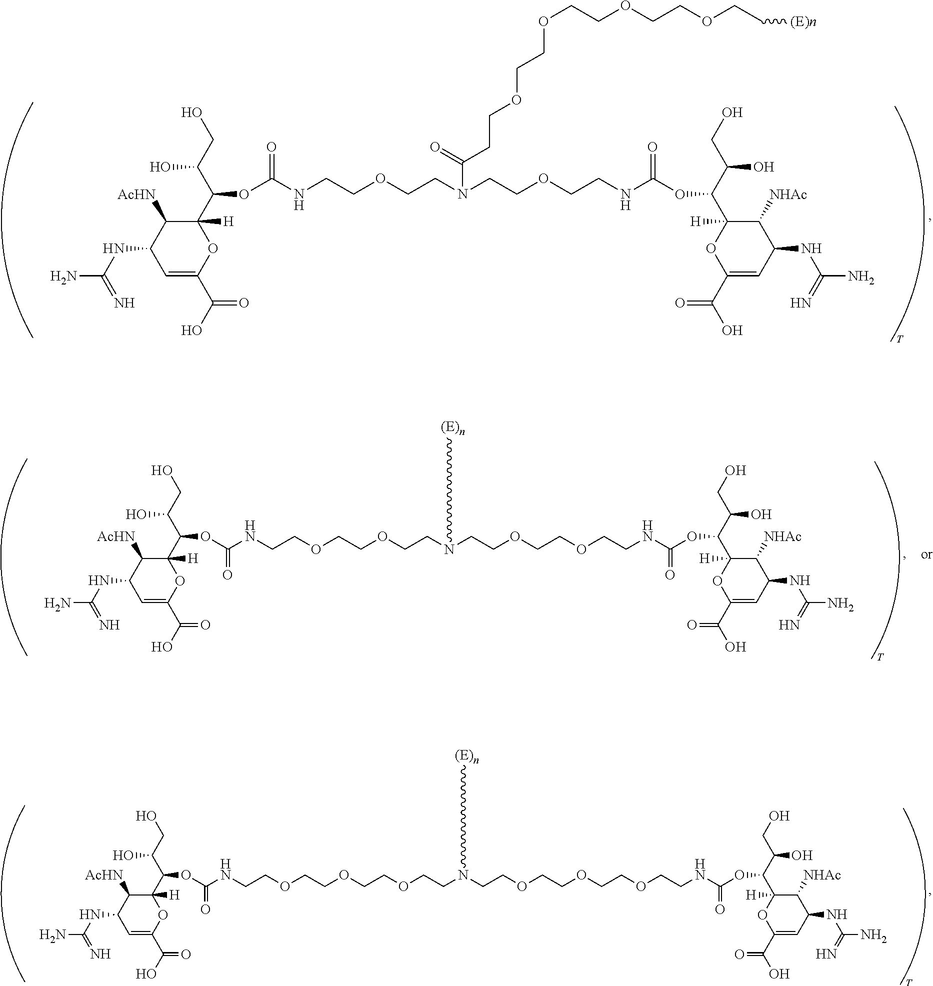

or a pharmaceutically acceptable salt thereof.

In some embodiments, the conjugate has the structure selected from:

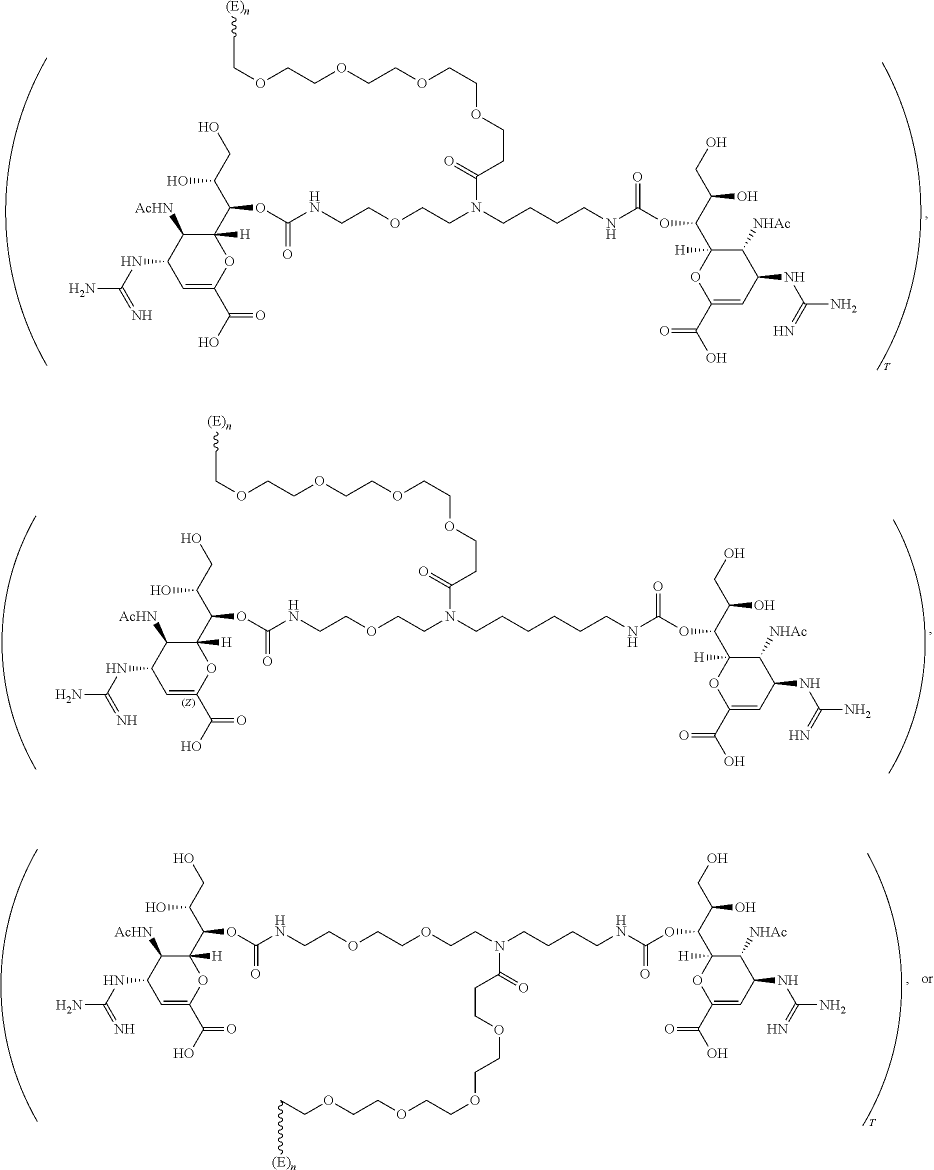

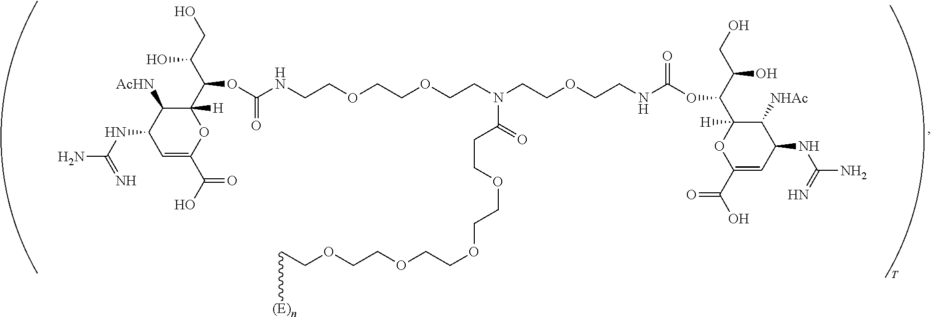

or a pharmaceutically acceptable salt thereof.

In some embodiments, the conjugate is described by formula (D-II-6):

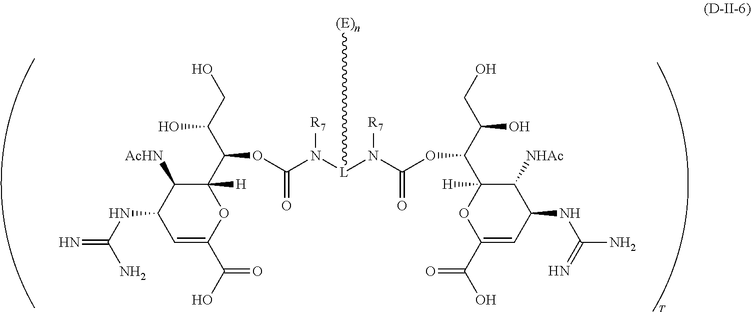

wherein R₇ is selected from H, C1-C20 alkyl, C3-C20 cycloalkyl, C3-C20 heterocycloalkyl; C5-C15 aryl, and C2-C15 heteroaryl; or a pharmaceutically acceptable salt thereof. In some embodiments, R₇ is selected from C1-C20 alkyl (e.g., methyl, ethyl, propyl, or butyl).

In some embodiments, the conjugate is described by formula (D-II-7):

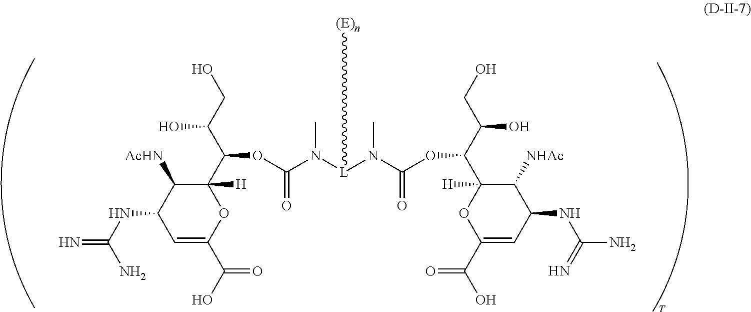

or a pharmaceutically acceptable salt thereof.

In some embodiments, the conjugate is described by formula (D-II-8):

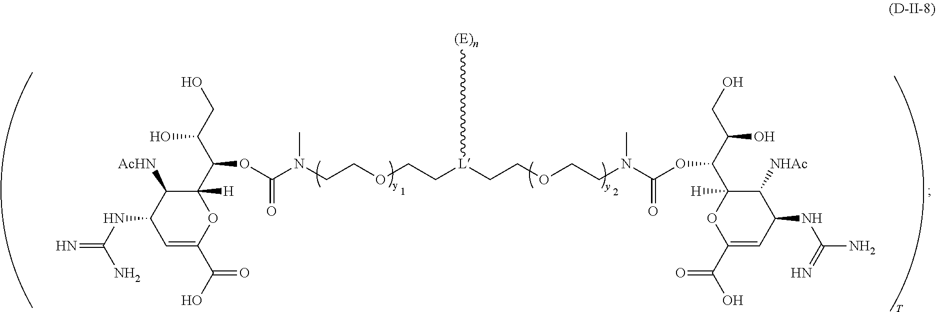

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure

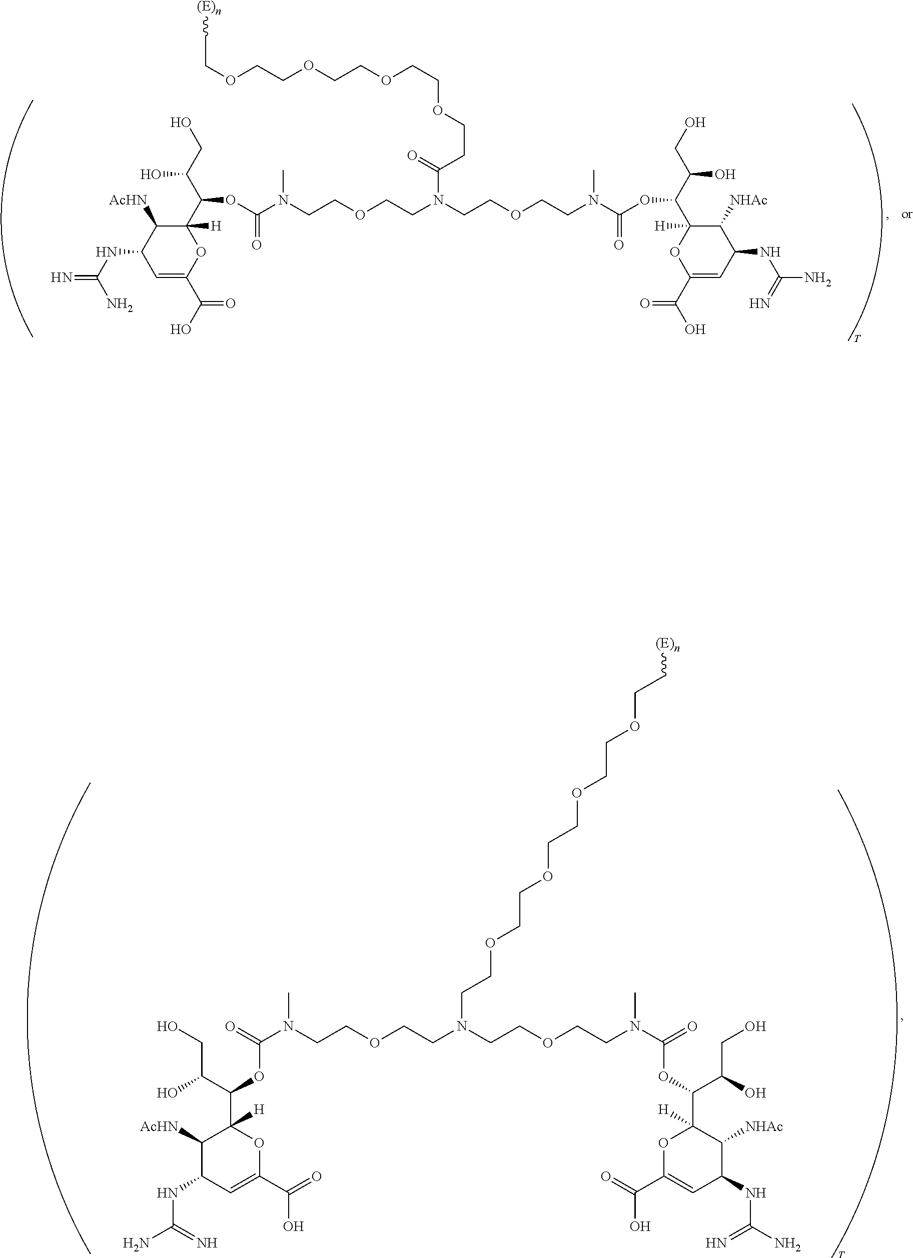

or a pharmaceutically acceptable salt thereof.

In some embodiments, the conjugate has the structure

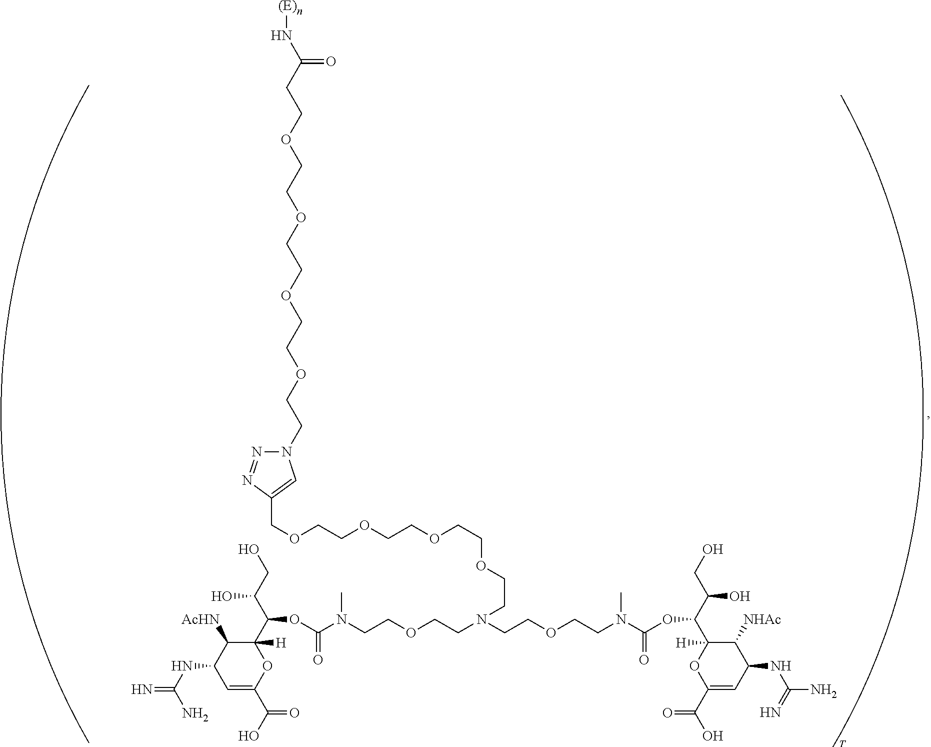

or a pharmaceutically acceptable salt thereof.

In some embodiments, the conjugate has the structure

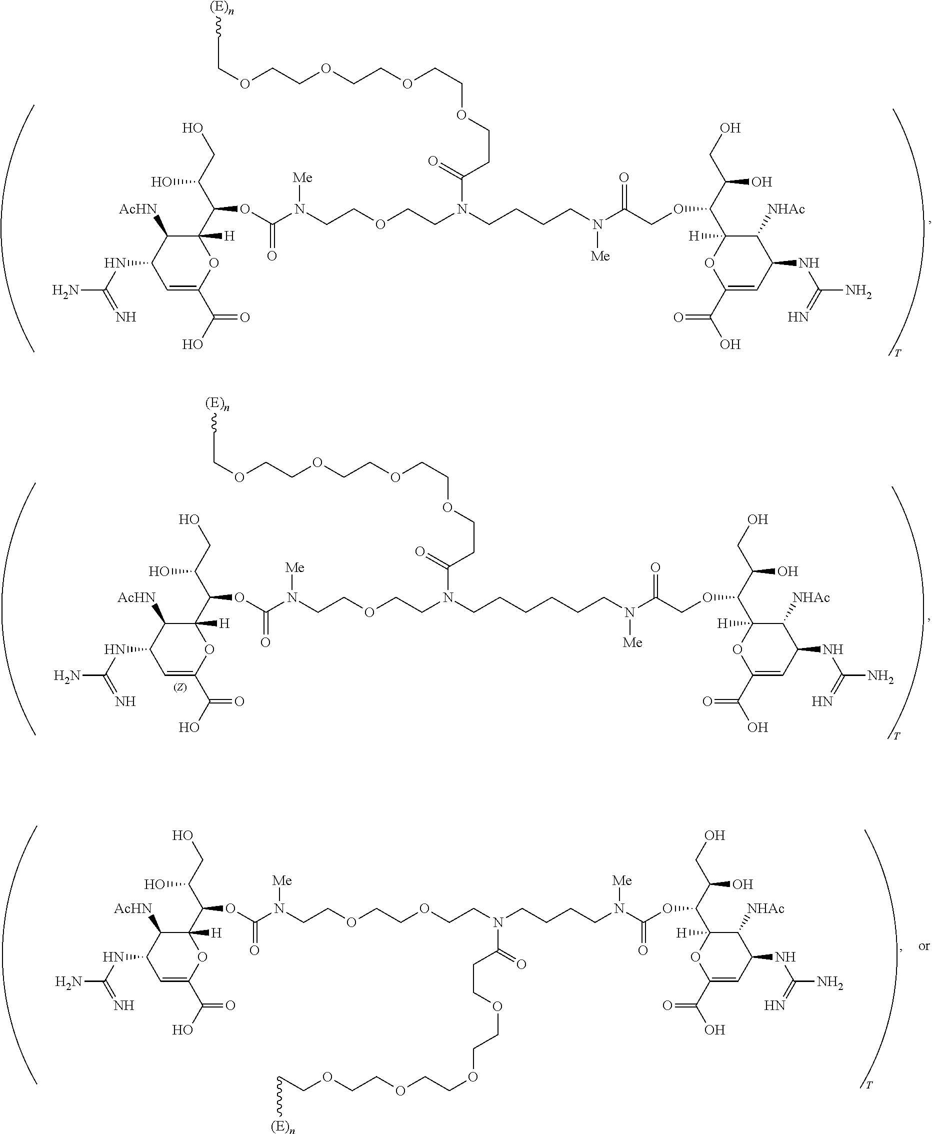

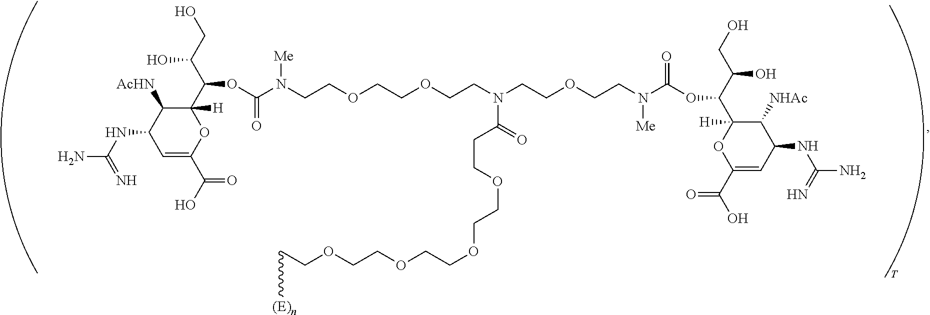

or a pharmaceutically acceptable salt thereof.

In some embodiments, the conjugate has the structure

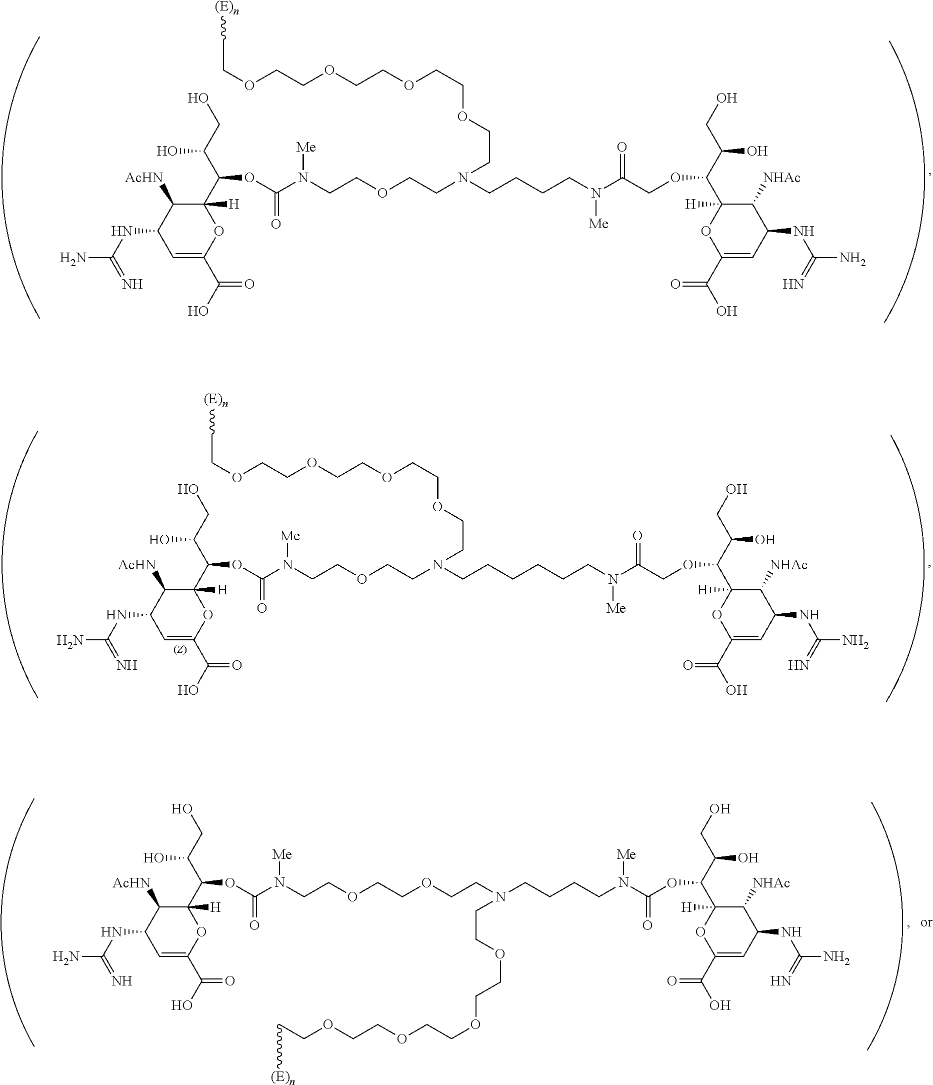

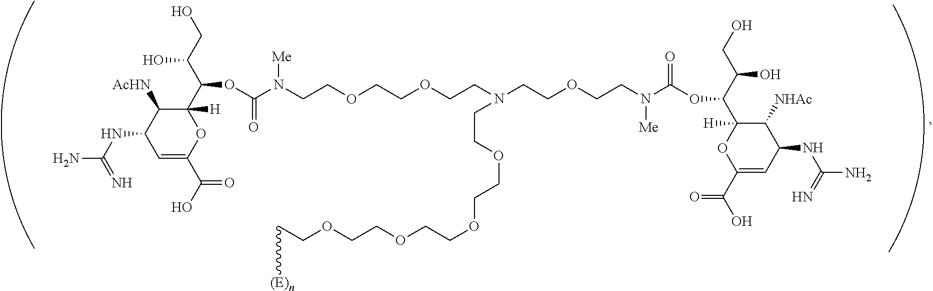

or a pharmaceutically acceptable salt thereof.

In some embodiments, the conjugate is described by formula (D-II-9):

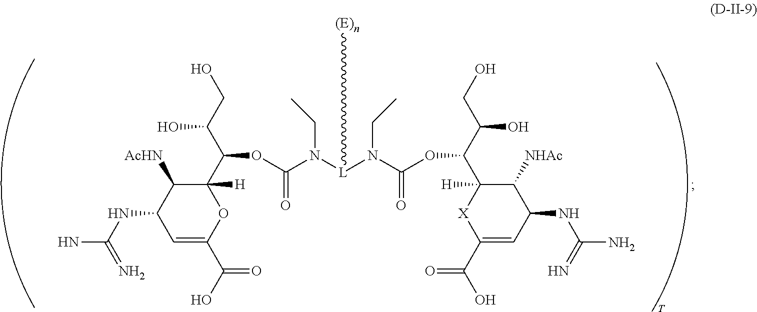

or a pharmaceutically acceptable salt thereof.

In some embodiments, the conjugate is described by formula (D-II-10):

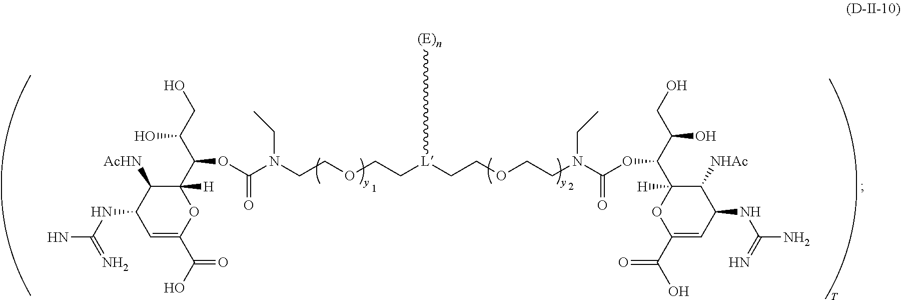

wherein L′ is the remainder of L, and y₁ and y₂ are each independently an integer from 1-20 (e.g., y₁ and y₂ are each independently 1, 2, 3, 4, 5, 6, 7, 8, 9, 10, 11, 12, 13, 14, 15, 16, 17, 18, 19, or 20), or a pharmaceutically acceptable salt thereof. In some embodiments, L′ is a nitrogen atom.

In some embodiments, the conjugate has the structure

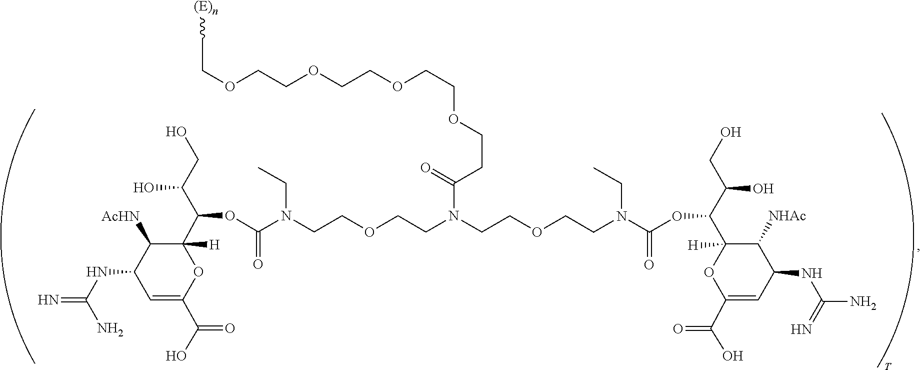

or a pharmaceutically acceptable salt thereof.

In some embodiments, the conjugate is described by formula (D-III):

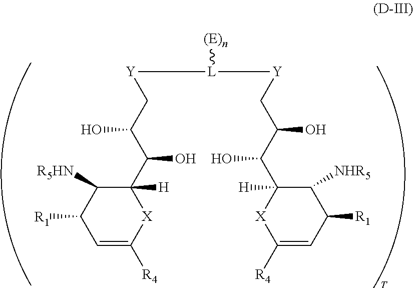

or a pharmaceutically acceptable salt thereof.

In some embodiments, the conjugate is described by formula (D-IV):

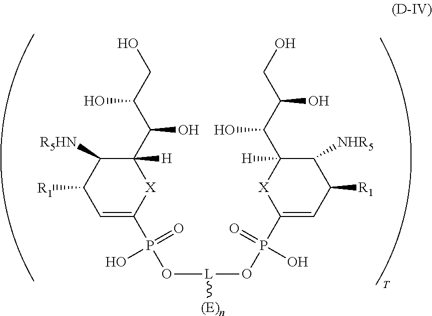

or a pharmaceutically acceptable salt thereof.

In some embodiments, the conjugate is described by formula (D-V):

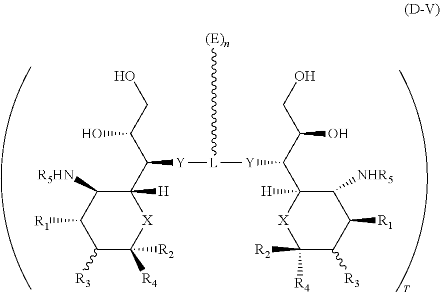

or a pharmaceutically acceptable salt thereof.

In some embodiments, the conjugate is described by formula (D-VI):

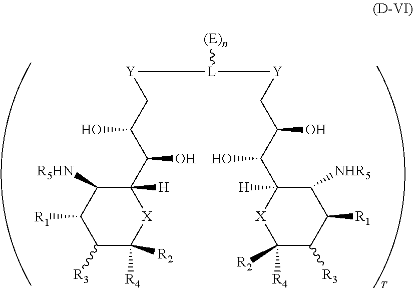

or a pharmaceutically acceptable salt thereof.

In some embodiments, the conjugate is described by formula (D-VII):

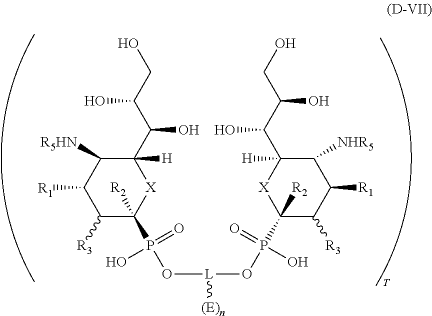

or a pharmaceutically acceptable salt thereof.

In some embodiments, the conjugate is described by formula (D-VIII):

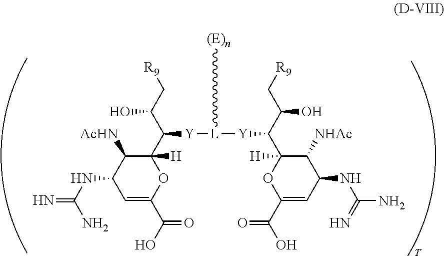

or a pharmaceutically acceptable salt thereof.

In some embodiments, the conjugate is any conjugate described by WO 2020/051498 and WO 2021/046549, each of which is hereby incorporated by reference.

In some embodiments of any of the aspects described herein, R₁ is OH. In some embodiments of any of the aspects described herein, R₁ is NH₂. In some embodiments of any of the aspects described herein, R₁ is —NHC(═NH)NH₂. In some embodiments of any of the aspects described herein, R₂ is —F. In some embodiments of any of the aspects described herein, R₃ is —F. In some embodiments of any of the aspects described herein, R₄ is —CO₂H. In some embodiments of any of the aspects described herein, R₅ is —COCH₃.

In some embodiments of any of the aspects described herein, L or L′ includes one or more optionally substituted C1-C20 alkylene, optionally substituted C1-C20 heteroalkylene, optionally substituted C2-C20 alkenylene, optionally substituted C2-C20 heteroalkenylene, optionally substituted C2-C20 alkynylene, optionally substituted C2-C20 heteroalkynylene, optionally substituted C3-C20 cycloalkylene, optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene, optionally substituted C2-C15 heteroarylene, O, S, NRⁱ, P, carbonyl, thiocarbonyl, sulfonyl, phosphate, phosphoryl, or imino, wherein Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl.

In some embodiments of any of the aspects described herein, L or L′ is oxo substituted. In some embodiments, the backbone of L or L′ comprises no more than 250 atoms. In some embodiments, L or L′ is capable of forming an amide, a carbamate, a sulfonyl, or a urea linkage. In some embodiments L or L′ is a bond. In some embodiments, L or L′ is an atom. In some embodiments, L or L′ is a nitrogen atom.

In some embodiments of any of the aspects described herein, L includes a polyethylene glycol (PEG) linker. A PEG linker includes a linker having the repeating unit structure (—CH₂CH₂O—)ₙ, wherein n is an integer from 2 to 100. A polyethylene glycol linker may covalently join a first anti-influenza moiety (e.g., neuraminidase inhibitor) and a second anti-influenza moiety (e.g., neuraminidase inhibitor) (e.g., in a conjugate of any one of formulas (D-I)-(D-VIII)). A polyethylene glycol linker may covalently join a anti-influenza moiety (e.g., neuraminidase inhibitor) dimer and E (e.g., in a conjugate of any one of formulas (D-I)-(D-VIII)). A polyethylene glycol linker may be selected any one of PEG₂ to PEG₁₀₀ (e.g., PEG₂, PEG₃, PEG₄, PEG₅, PEG₅-PEG₁₀, PEG₁₀-PEG₂₀, PEG₂₀-PEG₃₀, PEG₃₀-PEG₄₀, PEG₅₀-PEG₆₀, PEG₆₀-PEG₇₀, PEG₇₀-PEG₈₀, PEG₈₀-PEG₉₀, PEG₉₀-PEG₁₀₀). In some embodiments, L^{c} includes a PEG linker, where L^{c} is covalently attached to each of Q and E.

Intermediates of Table 1 may be conjugated to an Fc domain or Fc domain monomer (e.g., by way of a linker) by any suitable methods known to those of skill in the art, including any of the methods described or exemplified herein. In some embodiments, one or more nitrogen atoms of one or more surface exposed lysine residues of E or one or more sulfur atoms of one or more surface exposed cysteines in E is covalently conjugated to a linker (e.g., a PEG₂-PEG₂₀ linker). The linker conjugated to E may be functionalized such that it may react to form a covalent bond with any of the Ints described herein (e.g., an Int of Table 1). In some embodiments, E is conjugated to a linker functionalized with an azido group and the Int (e.g., an Int of Table 1) is functionalized with an alkyne group. Conjugation (e.g., by click chemistry) of the linker-azido of E and linker-alkyne of the Int forms a conjugate of the disclosure, for example a conjugate described by formula (5). In yet other embodiments, E is conjugated to a linker functionalized with an alkyne group and the Int (e.g., an Int of Table 1) is functionalized with an azido group. Conjugation of the linker-alkyne of E and linker-azido of the Int forms a conjugate of the disclosure, for example a conjugate described by any one of formulas (D-I)-(D-VIII). In yet other embodiments, the Int (e.g., an Int of Table 1) is functionalized with a phenyl ester group (e.g., a trifluorophenyl ester group or a tetrafluorophenyl ester group). Conjugation (e.g., by acylation) of E and the linker-phenyl ester (e.g., trifluorophenyl ester or tetrafluorophenyl ester) of the Int forms a conjugate of the invention, for example a conjugate described by any one of formulas (D-I)-(D-VIII). Conjugation (e.g., by acylation) of E and the linker-phenyl ester (e.g., trifluorophenyl ester or tetrafluorophenyl ester) of the Int is conducted, e.g., by methods described herein.

TABLE 1
Intermediates

| Inter- | |
|---|---|
| mediate | Structure |
| Int-1 | 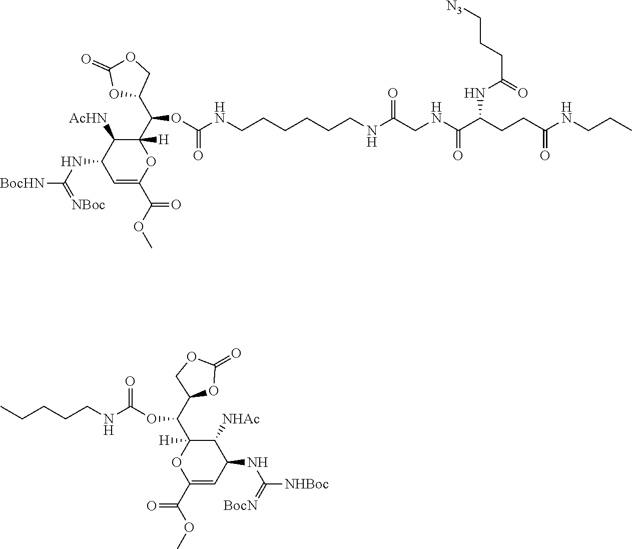 |
| Int-2 | 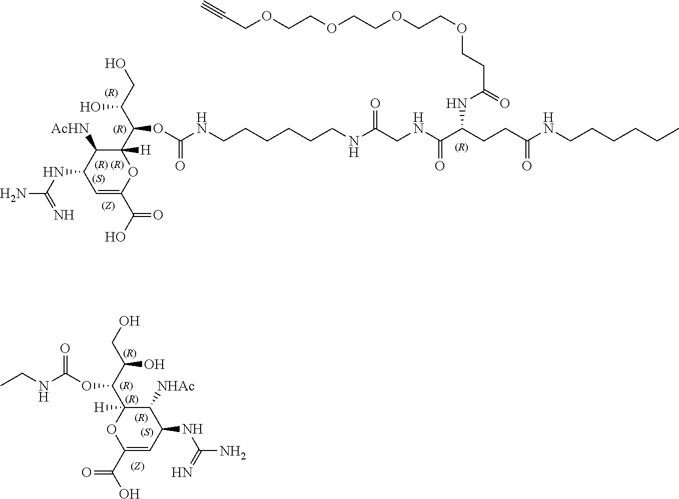 |
| Int-3 | 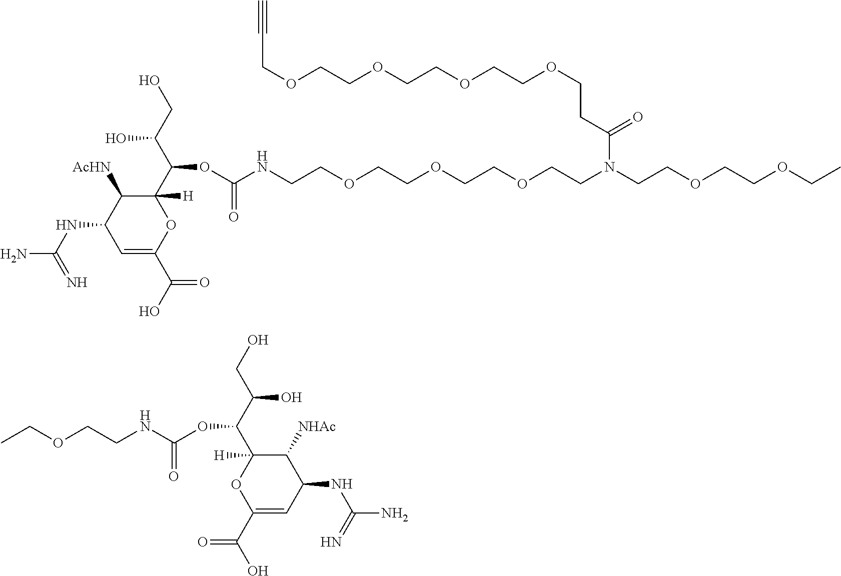 |
| Int-7 | 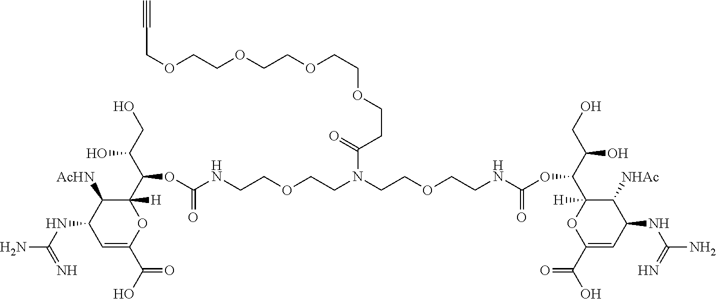 [SMILES: C#CCOCCOCCOCCOCCC(=O)N(CCOCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| Int-9 | 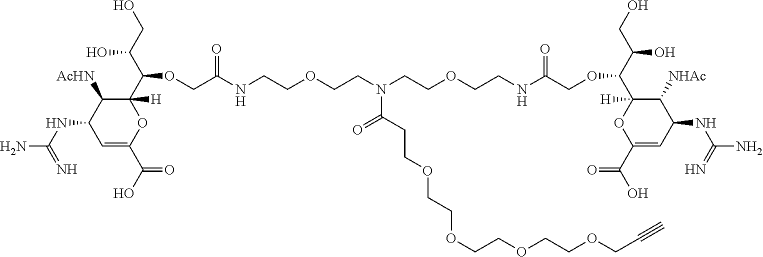 [SMILES: C#CCOCCOCCOCCOCCC(=O)N(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| Int-12 | 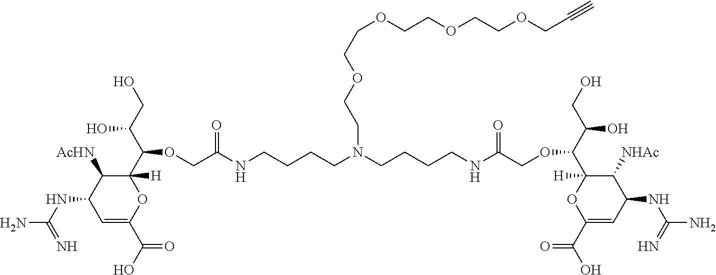 [SMILES: C#CCOCCOCCOCCOCCN(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| Int-17 | 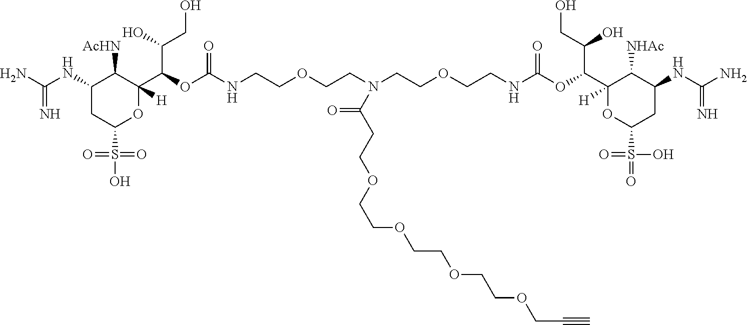 [SMILES: C#CCOCCOCCOCCOCCC(=O)N(CCOCCNC(=O)O[C@@H]([C@@H]1O[C@H](S(=O)(=O)O)C[C@H](NC(=N)N)[C@H]1NC(C)=O)[C@H](O)CO)CCOCCNC(=O)O[C@@H]([C@@H]1O[C@@H](S(=O)(=O)O)C[C@H](NC(=N)N)[C@H]1NC(C)=O)[C@H](O)CO] |
| Int-18 | 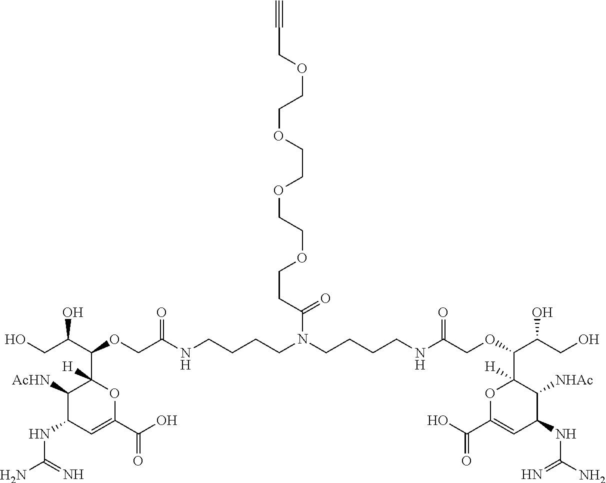 [SMILES: C#CCOCCOCCOCCOCCC(=O)N(CCCCNC(=O)CO[C@@H]([C@@H]1OC(C(=O)O)=C[C@H](NC(=N)N)[C@H]1NC(C)=O)[C@H](O)CO)CCCCNC(=O)CO[C@@H]([C@@H]1OC(C(=O)O)=C[C@H](NC(=N)N)[C@H]1NC(C)=O)[C@H](O)CO] |
| Int-19 | 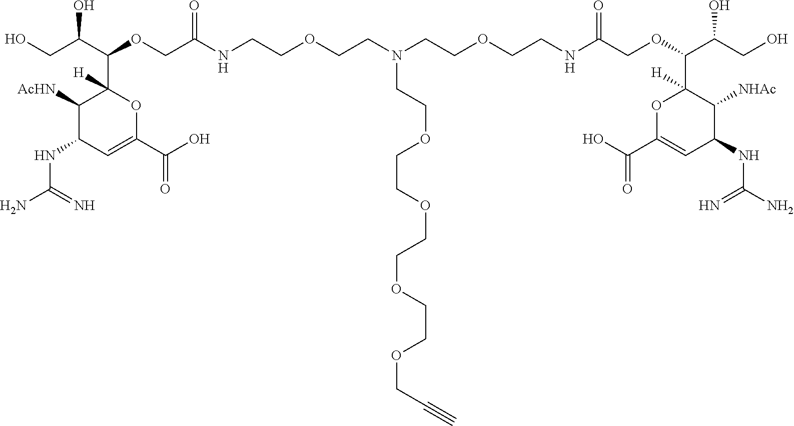 [SMILES: C#CCOCCOCCOCCOCCN(CCOCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| Int-20 | 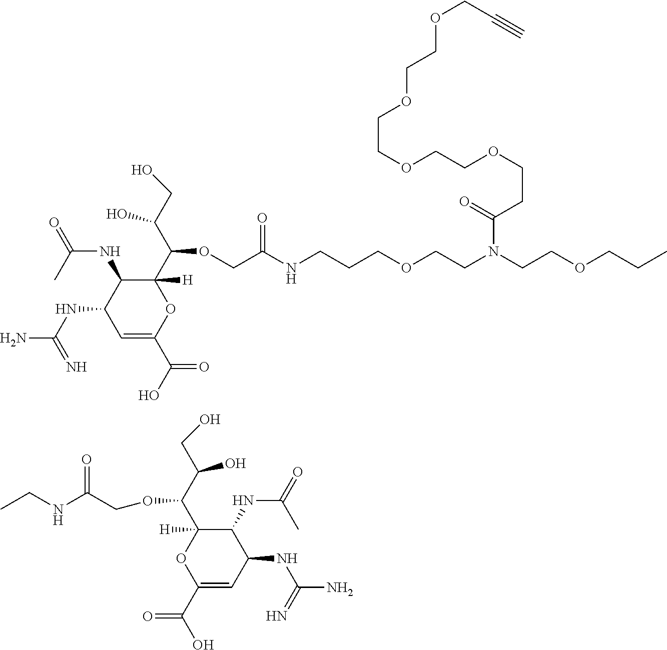 |
| Int-21 | 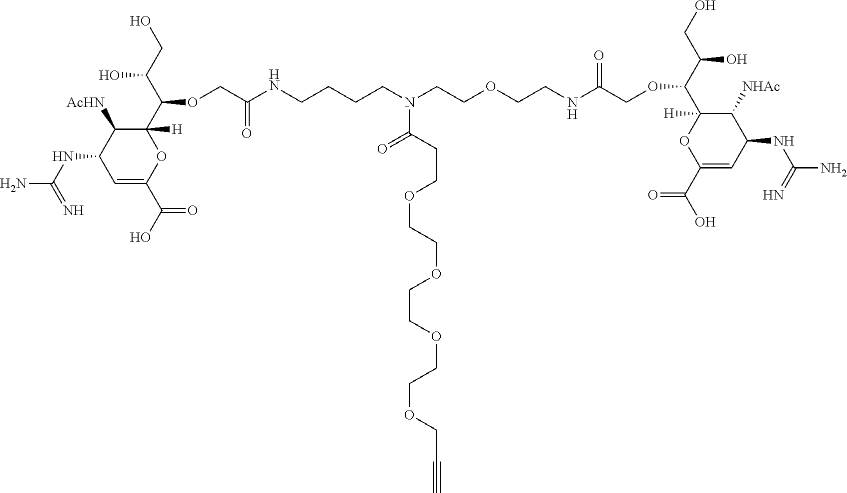 [SMILES: C#CCOCCOCCOCCOCCC(=O)N(CCCCNC(=O)CO[C@@H]([C@@H]1OC(C(=O)O)=C[C@H](NC(=N)N)[C@H]1NC(C)=O)[C@H](O)CO)CCOCCNC(=O)CO[C@@H]([C@@H]1OC(C(=O)O)=C[C@H](NC(=N)N)[C@H]1NC(C)=O)[C@H](O)CO] |
| Int-23 | 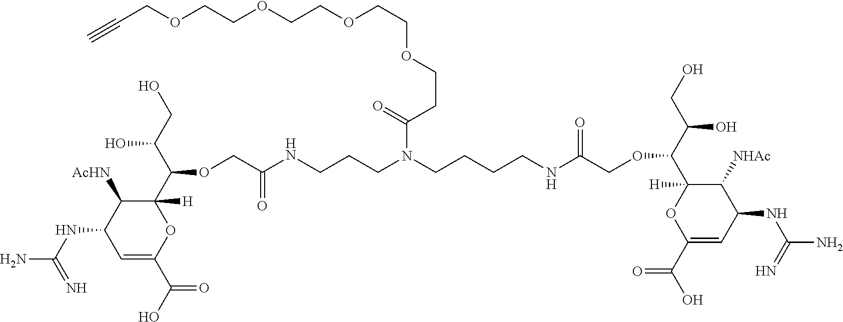 [SMILES: C#CCOCCOCCOCCOCCC(=O)N(CCCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| Int-24 | 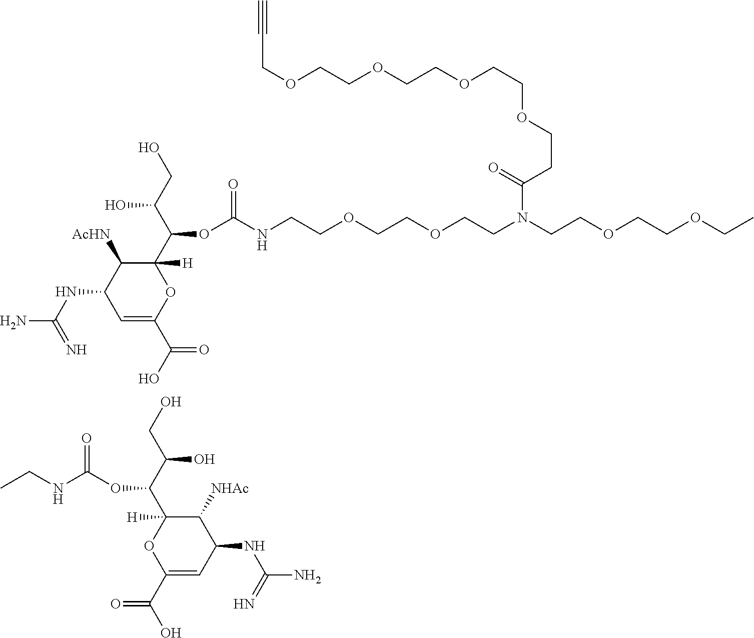 |
| Int-25 | 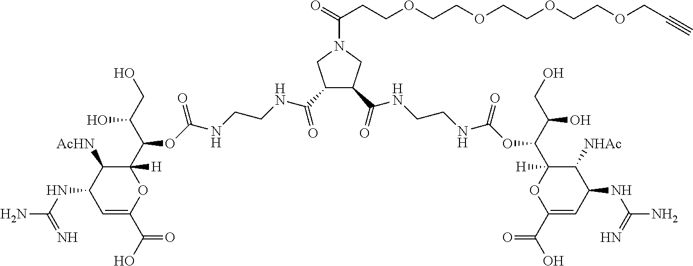 [SMILES: C#CCOCCOCCOCCOCCC(=O)N1C[C@@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| Int-26 | 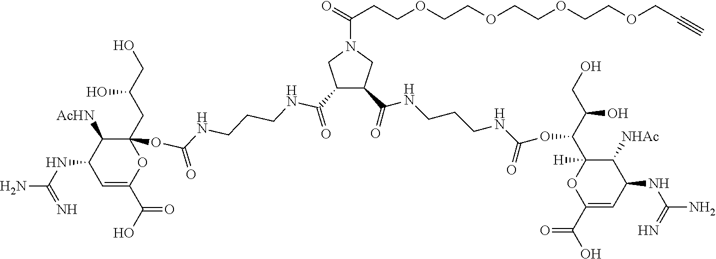 [SMILES: C#CCOCCOCCOCCOCCC(=O)N1C[C@@H](C(=O)NCCCNC(=O)O[C@@H]([C@@H]2OC(C(=O)O)=C[C@H](NC(=N)N)[C@H]2NC(C)=O)[C@H](O)CO)[C@H](C(=O)NCCCNC(=O)O[C@@]2(C[C@H](O)CO)OC(C(=O)O)=C[C@H](NC(=N)N)[C@H]2NC(C)=O)C1] |
| Int-27 | 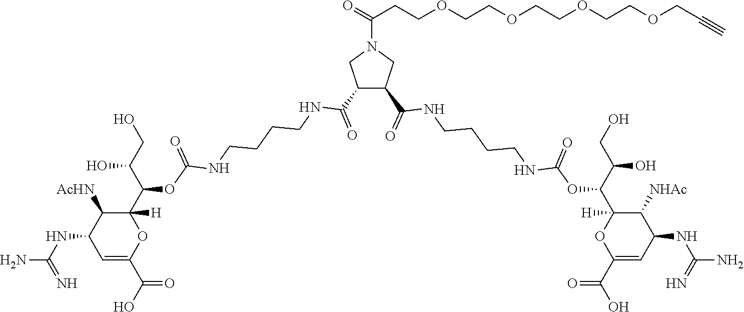 [SMILES: C#CCOCCOCCOCCOCCC(=O)N1C[C@@H](C(=O)NCCCCNC(=O)O[C@@H]([C@@H]2OC(C(=O)O)=C[C@H](NC(=N)N)[C@H]2NC(C)=O)[C@H](O)CO)[C@H](C(=O)NCCCCNC(=O)O[C@@H]([C@@H]2OC(C(=O)O)=C[C@H](NC(=N)N)[C@H]2NC(C)=O)[C@H](O)CO)C1] |
| Int-28 | 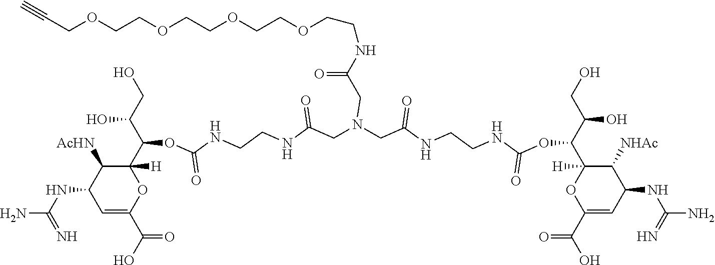 [SMILES: C#CCOCCOCCOCCOCCNC(=O)CN(CC(=O)NCCNC(=O)O[C@@H]([C@@H]1OC(C(=O)O)=C[C@H](NC(=N)N)[C@H]1NC(C)=O)[C@H](O)CO)CC(=O)NCCNC(=O)O[C@@H]([C@@H]1OC(C(=O)O)=C[C@H](NC(=N)N)[C@H]1NC(C)=O)[C@H](O)CO] |
| Int-31 | 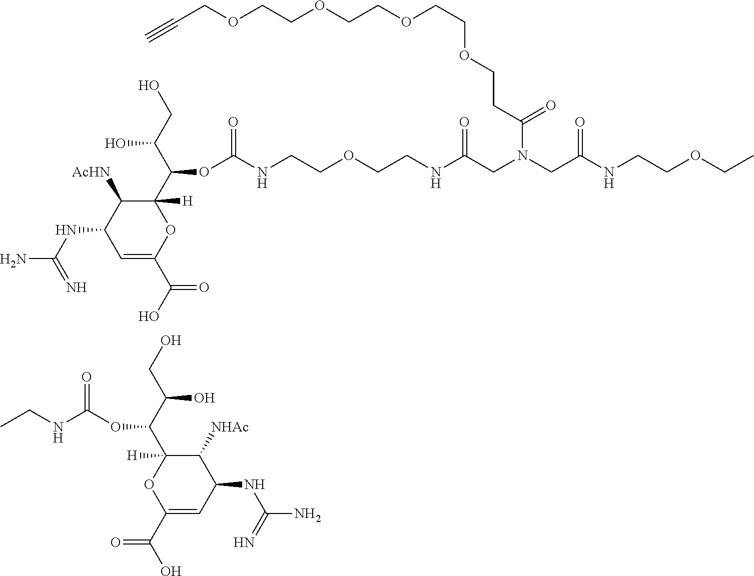 |
| Int-34 | 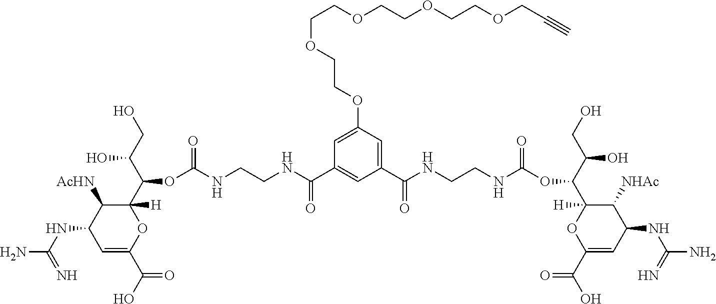 [SMILES: C#CCOCCOCCOCCOCCOc1cc(C(=O)NCCNC(=O)O[C@@H]([C@@H]2OC(C(=O)O)=C[C@H](NC(=N)N)[C@H]2NC(C)=O)[C@H](O)CO)cc(C(=O)NCCNC(=O)O[C@@H]([C@@H]2OC(C(=O)O)=C[C@H](NC(=N)N)[C@H]2NC(C)=O)[C@H](O)CO)c1] |
| Int-38 | 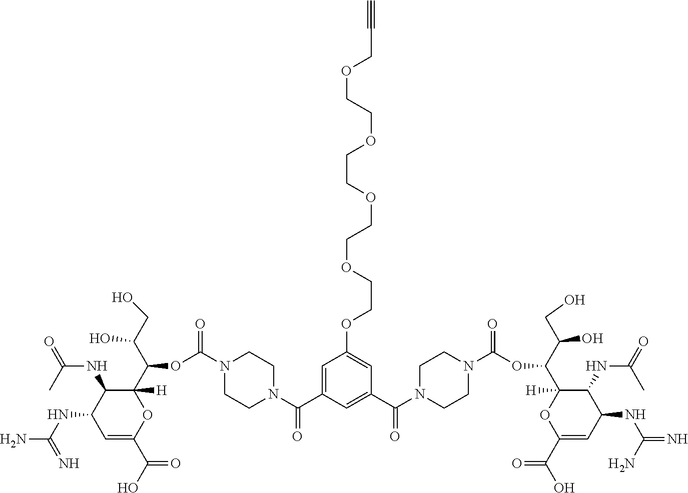 [SMILES: C#CCOCCOCCOCCOCCOc1cc(C(=O)N2CCN(C(=O)O[C@@H]([C@@H]3OC(C(=O)O)=C[C@H](NC(=N)N)[C@H]3NC(C)=O)[C@H](O)CO)CC2)cc(C(=O)N2CCN(C(=O)O[C@@H]([C@@H]3OC(C(=O)O)=C[C@H](NC(=N)N)[C@H]3NC(C)=O)[C@H](O)CO)CC2)c1] |
| Int-39 | 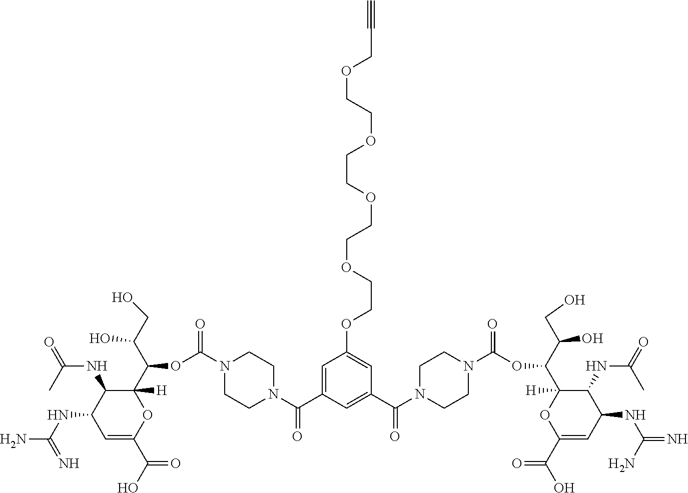 [SMILES: C#CCOCCOCCOCCOCCOc1cc(C(=O)N2CCN(C(=O)O[C@@H]([C@@H]3OC(C(=O)O)=C[C@H](NC(=N)N)[C@H]3NC(C)=O)[C@H](O)CO)CC2)cc(C(=O)N2CCN(C(=O)O[C@@H]([C@@H]3OC(C(=O)O)=C[C@H](NC(=N)N)[C@H]3NC(C)=O)[C@H](O)CO)CC2)c1] |
| Int-42 | 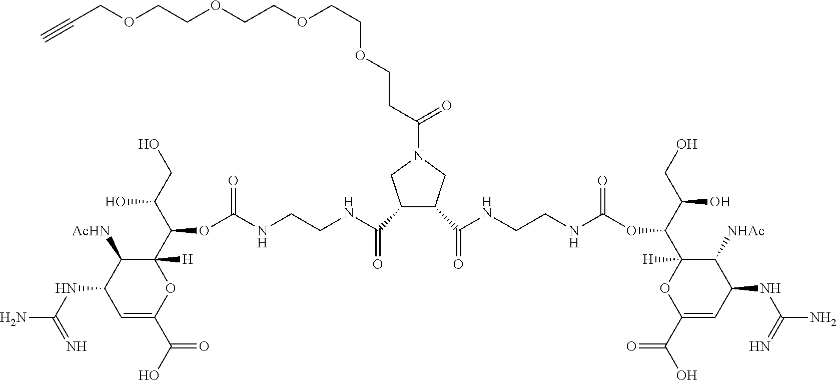 [SMILES: C#CCOCCOCCOCCOCCC(=O)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| Int-43 | 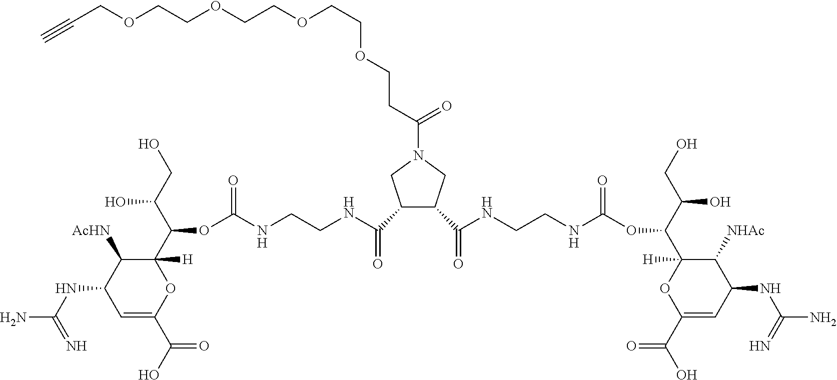 [SMILES: C#CCOCCOCCOCCOCCC(=O)N1C[C@H](C(=O)NCCNC(=O)O[C@@H]([C@@H]2OC(C(=O)O)=C[C@H](NC(=N)N)[C@H]2NC(C)=O)[C@H](O)CO)[C@H](C(=O)NCCNC(=O)O[C@@H]([C@@H]2OC(C(=O)O)=C[C@H](NC(=N)N)[C@H]2NC(C)=O)[C@H](O)CO)C1] |
| Int-44 | 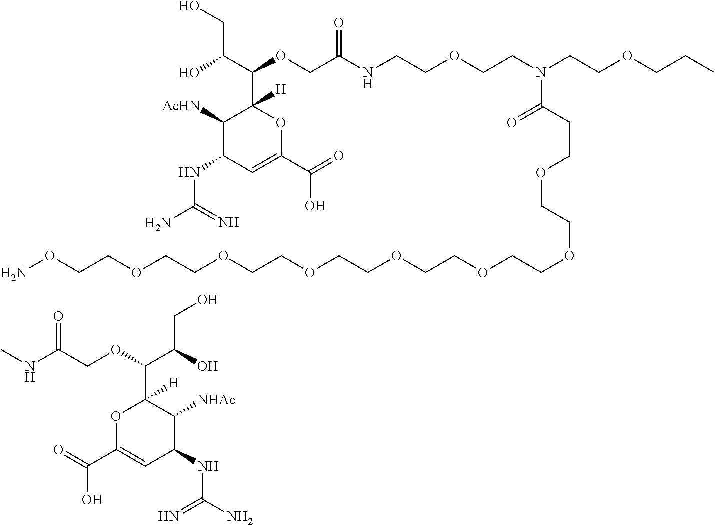 |
| Int-45 | 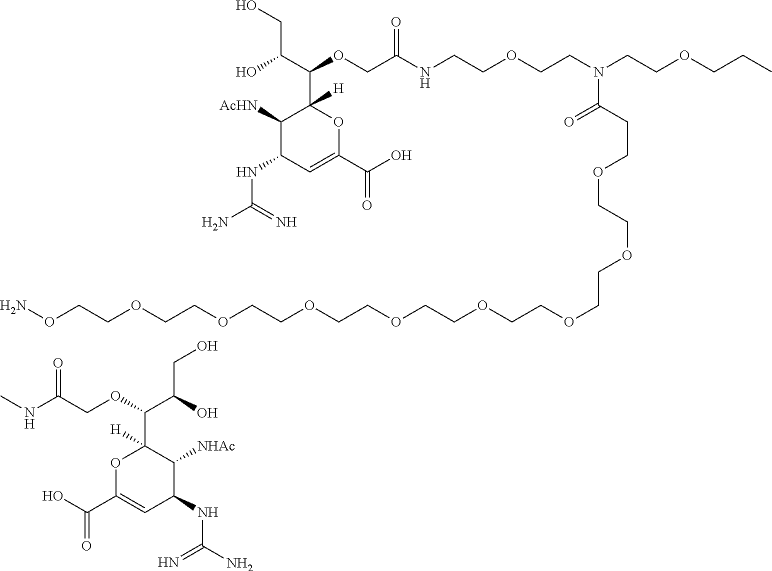 |
| Int-46 | 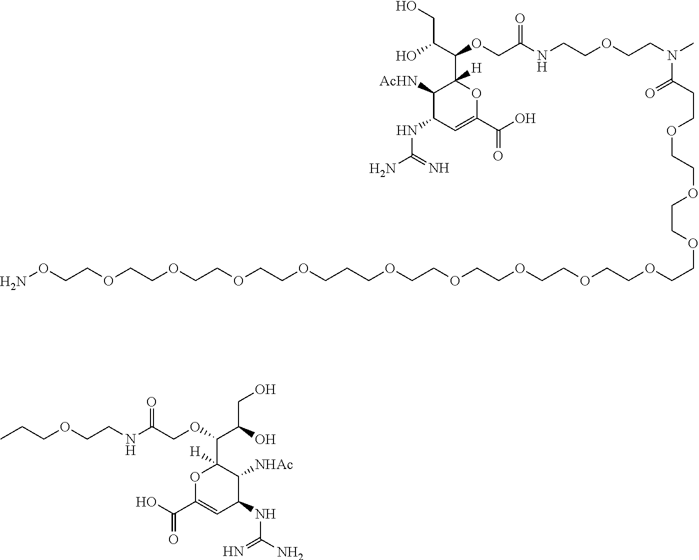 |
| Int-47 | 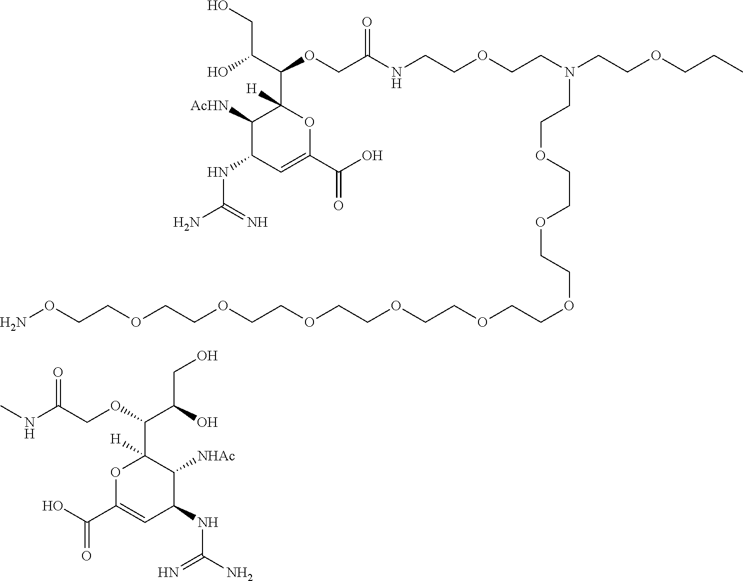 |
| Int-48 | 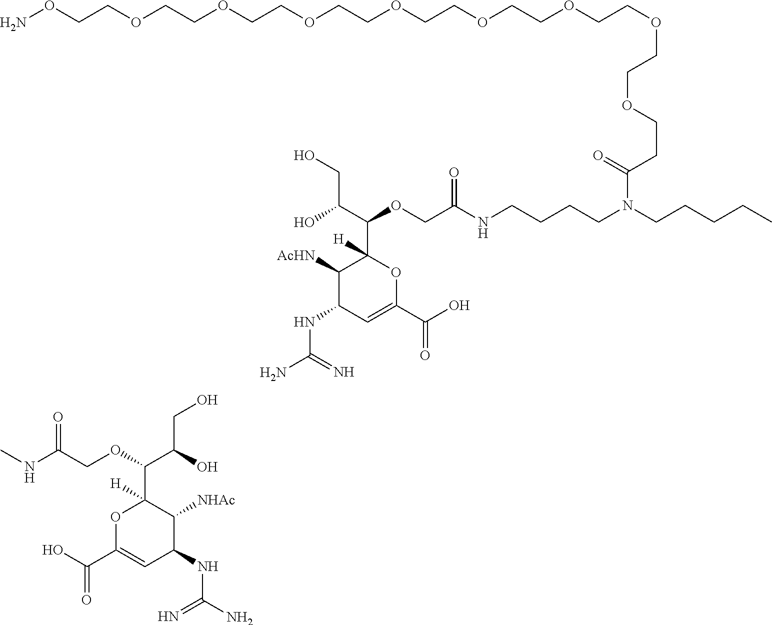 |
| Int-49 | 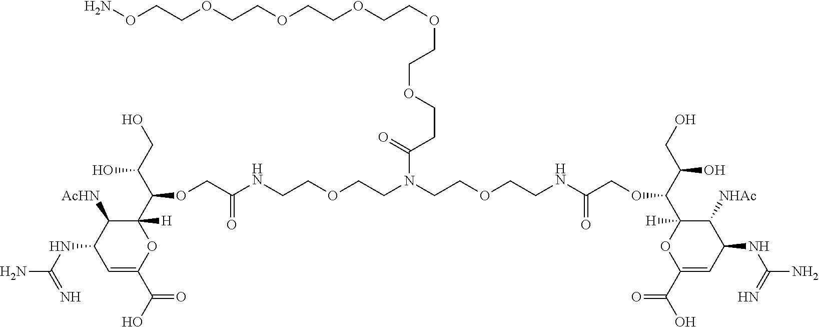 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCON)[C@H](O)CO)OC(C(=O)O)=C[C@@H]1NC(=N)N] |
| Int-50 | 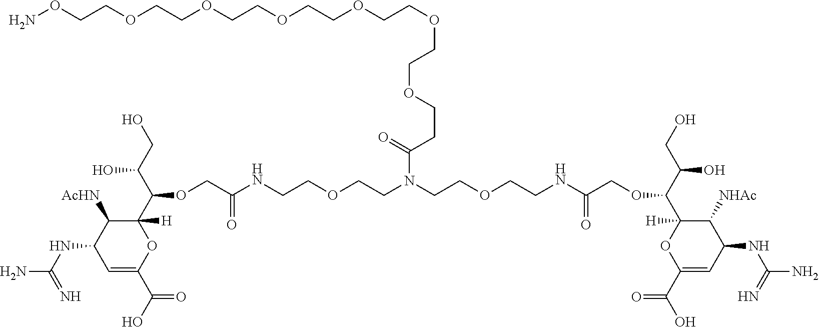 [SMILES: CC(=O)N[C@H]1[C@H]([C@H](OCC(=O)NCCOCCN(CCOCCNC(=O)CO[C@@H]([C@@H]2OC(C(=O)O)=C[C@H](NC(=N)N)[C@H]2NC(C)=O)[C@H](O)CO)C(=O)CCOCCOCCOCCOCCOCCOCCON)[C@H](O)CO)OC(C(=O)O)=C[C@@H]1NC(=N)N] |
| Int-52 | 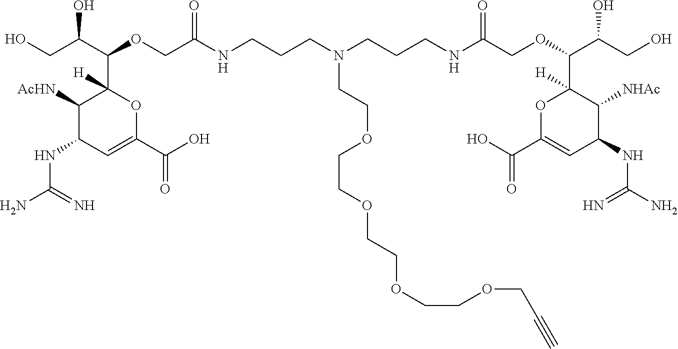 [SMILES: C#CCOCCOCCOCCOCCN(CCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| Int-53 | 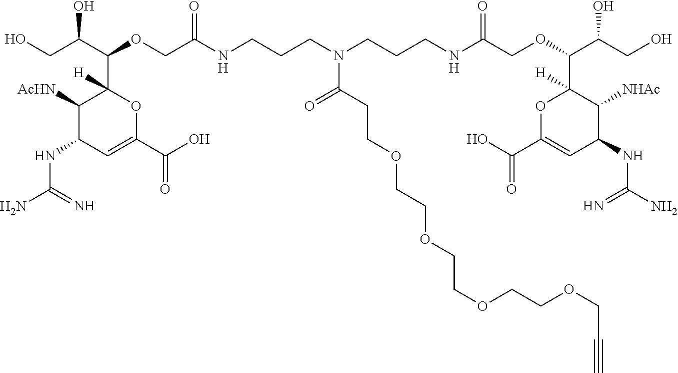 [SMILES: C#CCOCCOCCOCCOCCC(=O)N(CCCNC(=O)CO[C@@H]([C@@H]1OC(C(=O)O)=C[C@H](NC(=N)N)[C@H]1NC(C)=O)[C@H](O)CO)CCCNC(=O)CO[C@@H]([C@@H]1OC(C(=O)O)=C[C@H](NC(=N)N)[C@H]1NC(C)=O)[C@H](O)CO] |
| Int-54 | 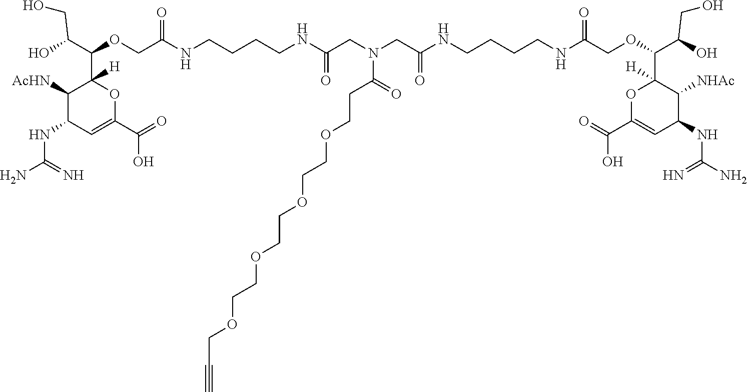 [SMILES: C#CCOCCOCCOCCOCCC(=O)N(CC(=O)NCCCCNC(=O)CO[C@@H]([C@@H]1OC(C(=O)O)=C[C@H](NC(=N)N)[C@H]1NC(C)=O)[C@H](O)CO)CC(=O)NCCCCNC(=O)CO[C@@H]([C@@H]1OC(C(=O)O)=C[C@H](NC(=N)N)[C@H]1NC(C)=O)[C@H](O)CO] |
| Int-55 | 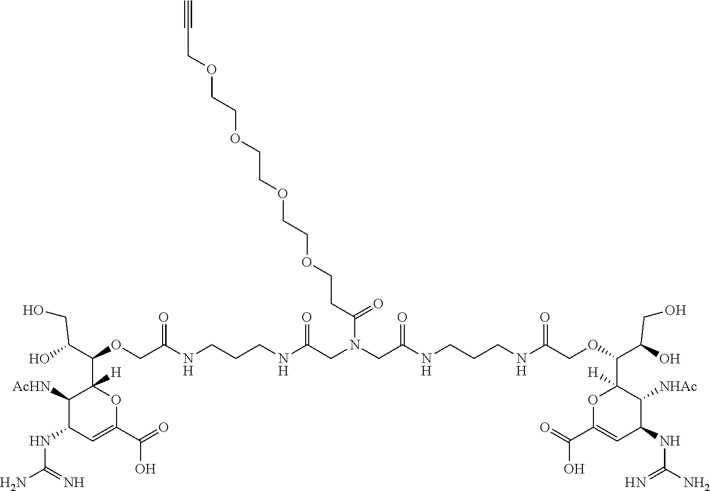 [SMILES: C#CCOCCOCCOCCOCCC(=O)N(CC(=O)NCCCNC(=O)CO[C@@H]([C@@H]1OC(C(=O)O)=C[C@H](NC(=N)N)[C@H]1NC(C)=O)[C@H](O)CO)CC(=O)NCCCNC(=O)CO[C@@H]([C@@H]1OC(C(=O)O)=C[C@H](NC(=N)N)[C@H]1NC(C)=O)[C@H](O)CO] |
| Int-57 | 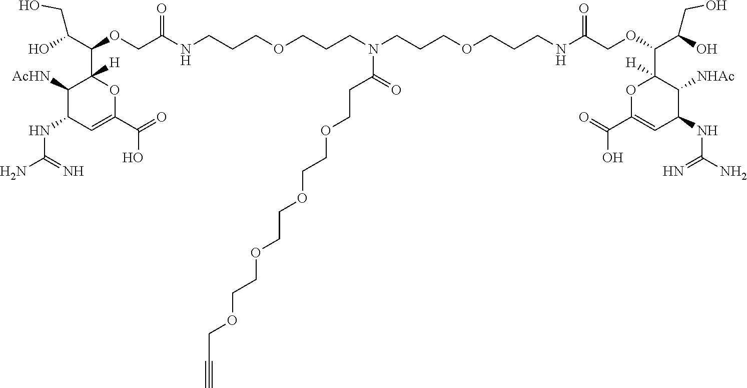 [SMILES: C#CCOCCOCCOCCOCCC(=O)N(CCCOCCCNC(=O)CO[C@@H]([C@@H]1OC(C(=O)O)=C[C@H](NC(=N)N)[C@H]1NC(C)=O)[C@H](O)CO)CCCOCCCNC(=O)CO[C@@H]([C@@H]1OC(C(=O)O)=C[C@H](NC(=N)N)[C@H]1NC(C)=O)[C@H](O)CO] |
| Int-58 | 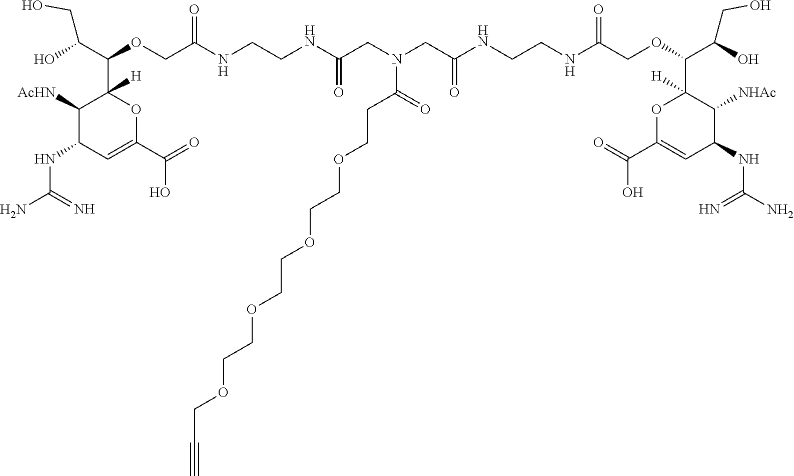 |
| Int-59 | 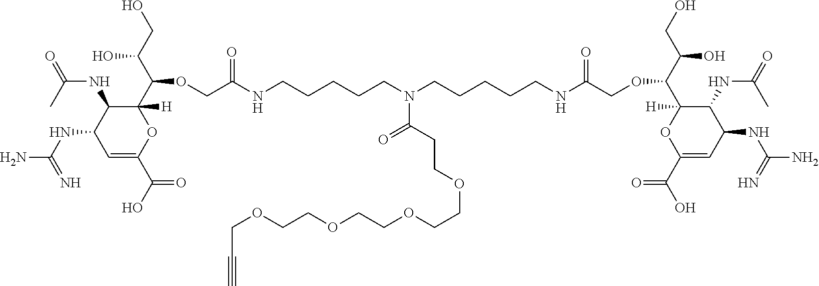 [SMILES: C#CCOCCOCCOCCOCCC(=O)N(CCCCCNC(=O)CO[C@@H]([C@@H]1OC(C(=O)O)=C[C@H](NC(=N)N)[C@H]1NC(C)=O)[C@H](O)CO)CCCCCNC(=O)CO[C@@H]([C@@H]1OC(C(=O)O)=C[C@H](NC(=N)N)[C@H]1NC(C)=O)[C@H](O)CO] |
| Int-60 | 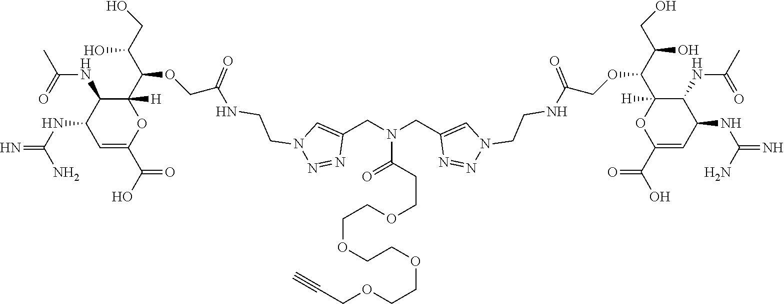 [SMILES: C#CCOCCOCCOCCOCCC(=O)N(Cc1cn(CCNC(=O)CO[C@@H]([C@@H]2OC(C(=O)O)=C[C@H](NC(=N)N)[C@H]2NC(C)=O)[C@H](O)CO)nn1)Cc1cn(CCNC(=O)CO[C@@H]([C@@H]2OC(C(=O)O)=C[C@H](NC(=N)N)[C@H]2NC(C)=O)[C@H](O)CO)nn1] |
| Int-61 | 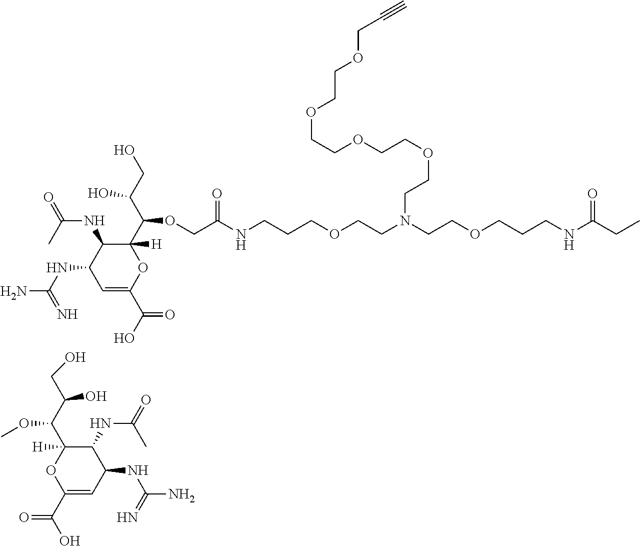 |
| Int-62 | 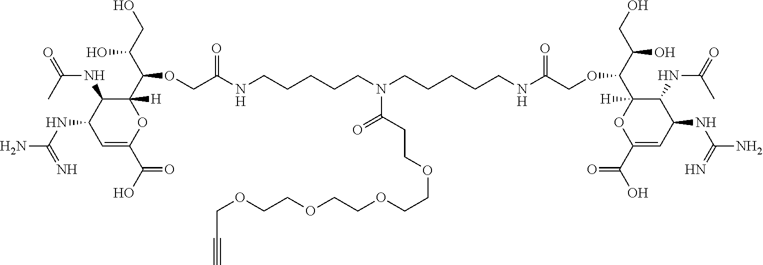 [SMILES: C#CCOCCOCCOCCOCCC(=O)N(CCCCCNC(=O)CO[C@@H]([C@@H]1OC(C(=O)O)=C[C@H](NC(=N)N)[C@H]1NC(C)=O)[C@H](O)CO)CCCCCNC(=O)CO[C@@H]([C@@H]1OC(C(=O)O)=C[C@H](NC(=N)N)[C@H]1NC(C)=O)[C@H](O)CO] |
| Int-63 | 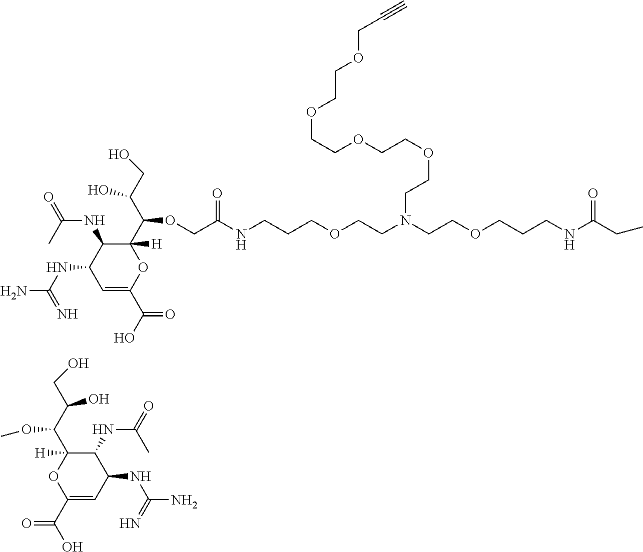 |
| Int-64 | 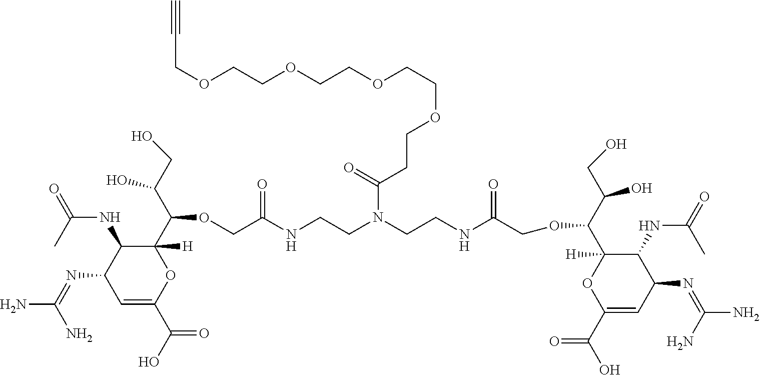 [SMILES: C#CCOCCOCCOCCOCCC(=O)N(CCNC(=O)CO[C@@H]([C@@H]1OC(C(=O)O)=C[C@H](N=C(N)N)[C@H]1NC(C)=O)[C@H](O)CO)CCNC(=O)CO[C@@H]([C@@H]1OC(C(=O)O)=C[C@H](N=C(N)N)[C@H]1NC(C)=O)[C@H](O)CO] |
| Int-65 | 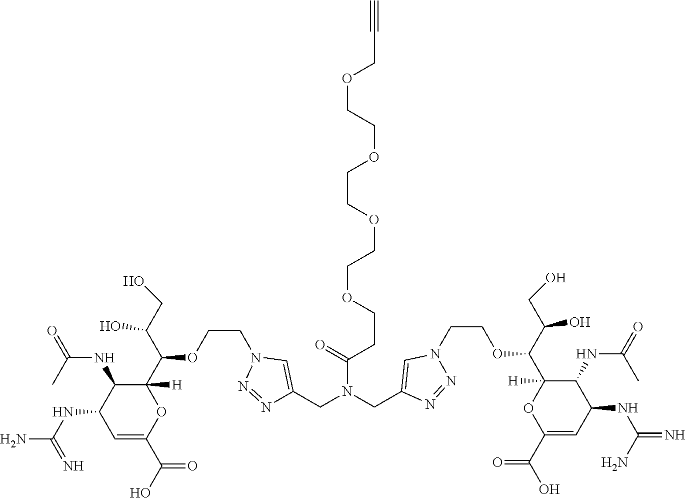 [SMILES: C#CCOCCOCCOCCOCCC(=O)N(Cc1cn(CCO[C@@H]([C@@H]2OC(C(=O)O)=C[C@H](NC(=N)N)[C@H]2NC(C)=O)[C@H](O)CO)nn1)Cc1cn(CCO[C@@H]([C@@H]2OC(C(=O)O)=C[C@H](NC(=N)N)[C@H]2NC(C)=O)[C@H](O)CO)nn1] |
| Int-69 | 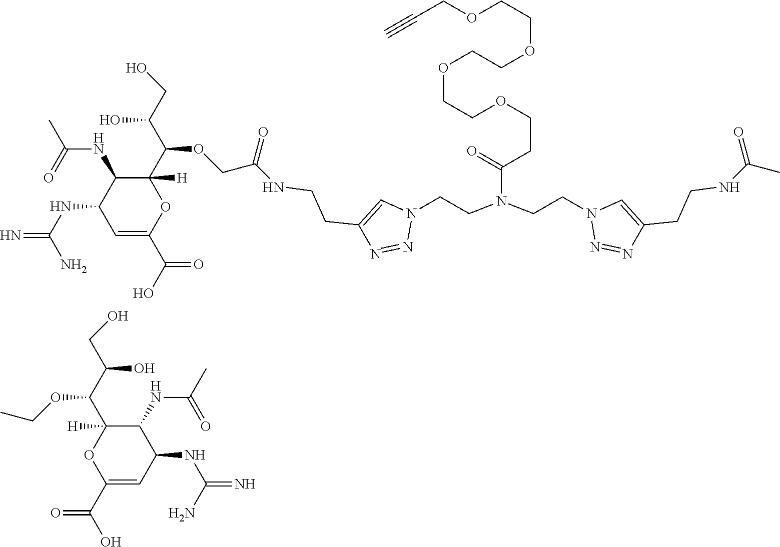 |
| Int-70 | 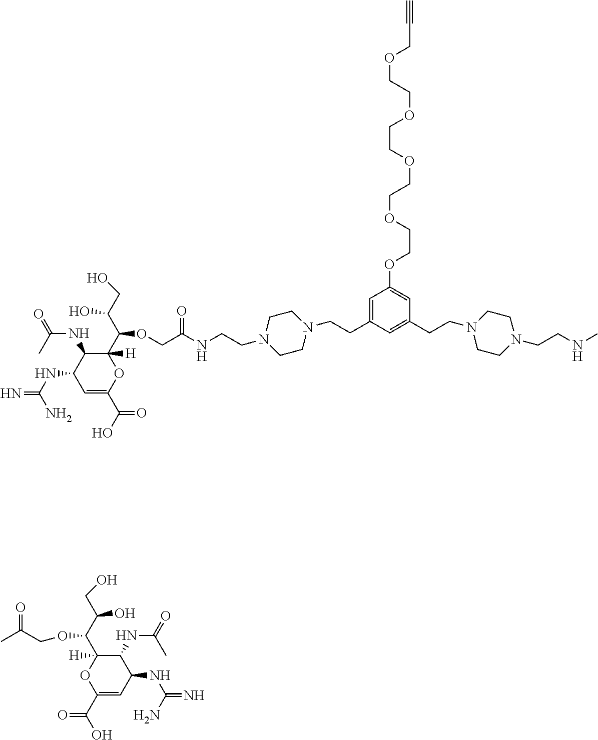 |
| Int-71 | 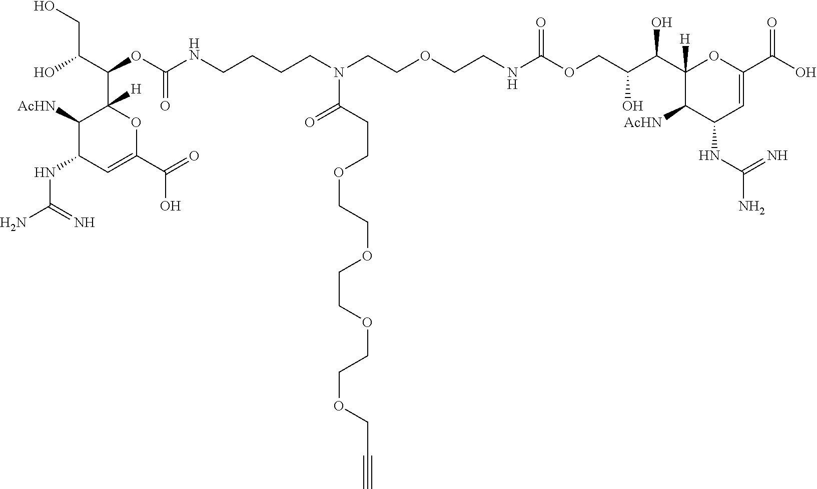 [SMILES: C#CCOCCOCCOCCOCCC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)OC[C@@H](O)[C@@H](O)[C@@H]1OC(C(=O)O)=C[C@H](NC(=N)N)[C@H]1NC(C)=O] |
| Int-72 | 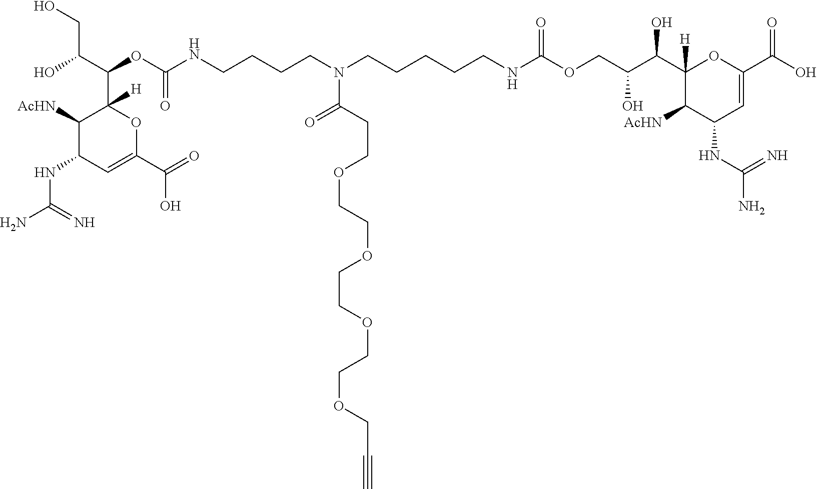 [SMILES: C#CCOCCOCCOCCOCCC(=O)N(CCCCCNC(=O)OC[C@@H](O)[C@@H](O)[C@@H]1OC(C(=O)O)=C[C@H](NC(=N)N)[C@H]1NC(C)=O)CCCCNC(=O)O[C@@H]([C@@H]1OC(C(=O)O)=C[C@H](NC(=N)N)[C@H]1NC(C)=O)[C@H](O)CO] |
| Int-73 | 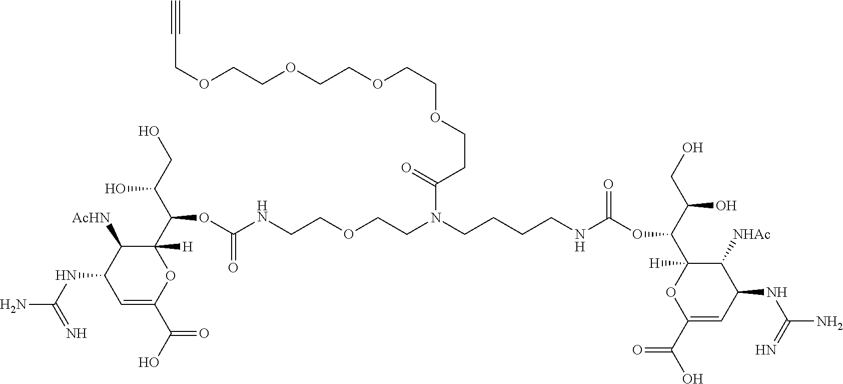 [SMILES: C#CCOCCOCCOCCOCCC(=O)N(CCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| Int-74 | 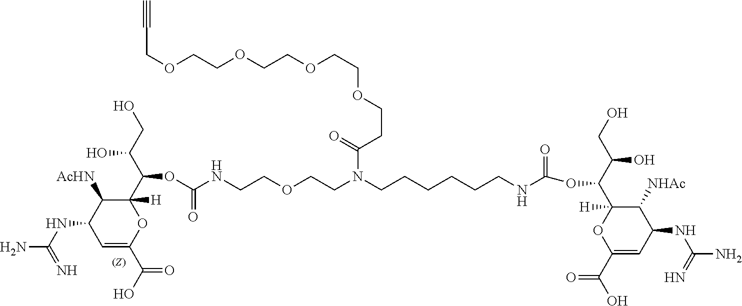 [SMILES: C#CCOCCOCCOCCOCCC(=O)N(CCCCCCNC(=O)O[C@@H]([C@@H]1OC(C(=O)O)=C[C@H](NC(=N)N)[C@H]1NC(C)=O)[C@H](O)CO)CCOCCNC(=O)O[C@@H]([C@@H]1OC(C(=O)O)=C[C@H](NC(=N)N)[C@H]1NC(C)=O)[C@H](O)CO] |
| Int-75 | 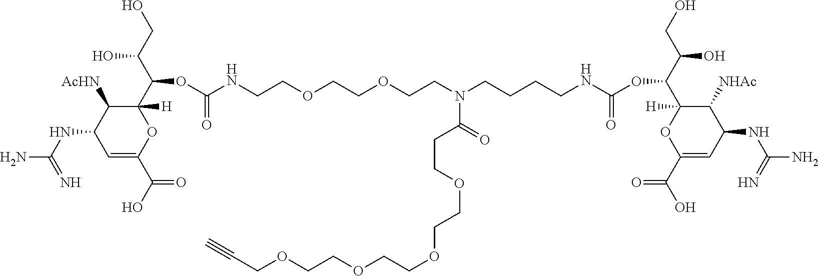 [SMILES: C#CCOCCOCCOCCOCCC(=O)N(CCCCNC(=O)O[C@@H]([C@@H]1OC(C(=O)O)=C[C@H](NC(=N)N)[C@H]1NC(C)=O)[C@H](O)CO)CCOCCOCCNC(=O)O[C@@H]([C@@H]1OC(C(=O)O)=C[C@H](NC(=N)N)[C@H]1NC(C)=O)[C@H](O)CO] |
| Int-76 | 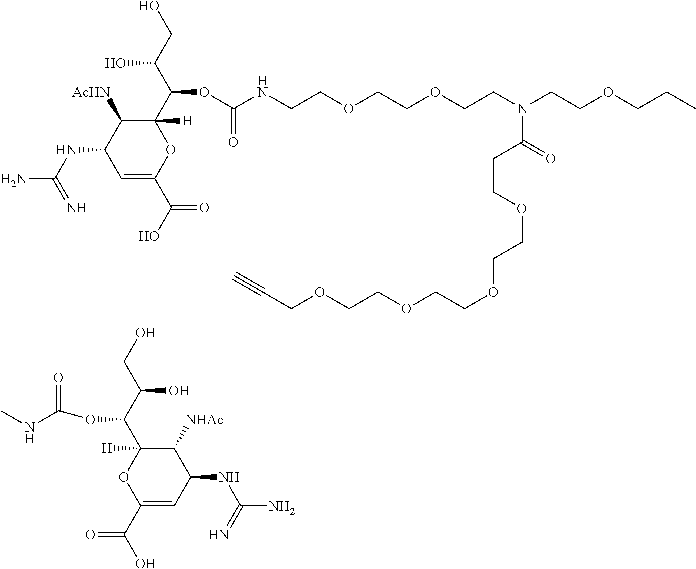 |
| Int-77 | 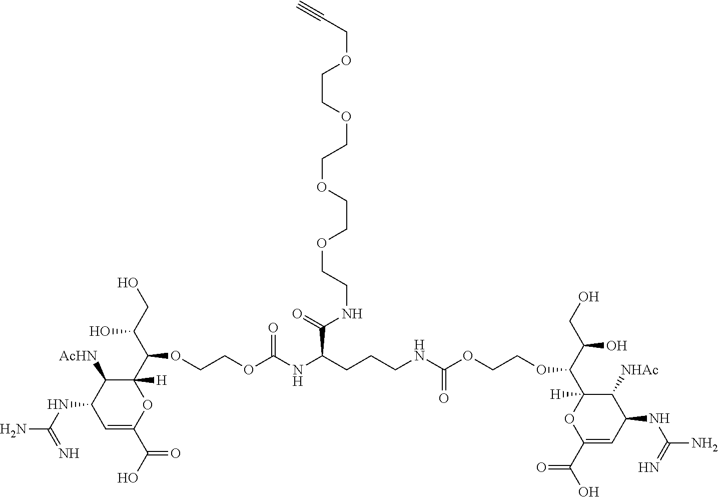 [SMILES: C#CCOCCOCCOCCOCCNC(=O)[C@@H](CCCNC(=O)OCCO[C@@H]([C@@H]1OC(C(=O)O)=C[C@H](NC(=N)N)[C@H]1NC(C)=O)[C@H](O)CO)NC(=O)OCCO[C@@H]([C@@H]1OC(C(=O)O)=C[C@H](NC(=N)N)[C@H]1NC(C)=O)[C@H](O)CO] |
| Int-78 | 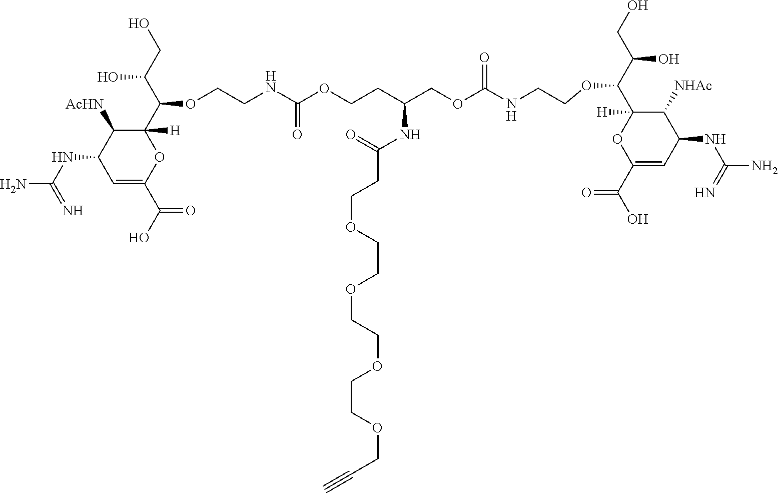 [SMILES: C#CCOCCOCCOCCOCCC(=O)N[C@@H](CCOC(=O)NCCO[C@@H]([C@@H]1OC(C(=O)O)=C[C@H](NC(=N)N)[C@H]1NC(C)=O)[C@H](O)CO)COC(=O)NCCO[C@@H]([C@@H]1OC(C(=O)O)=C[C@H](NC(=N)N)[C@H]1NC(C)=O)[C@H](O)CO] |
| Int-79 | 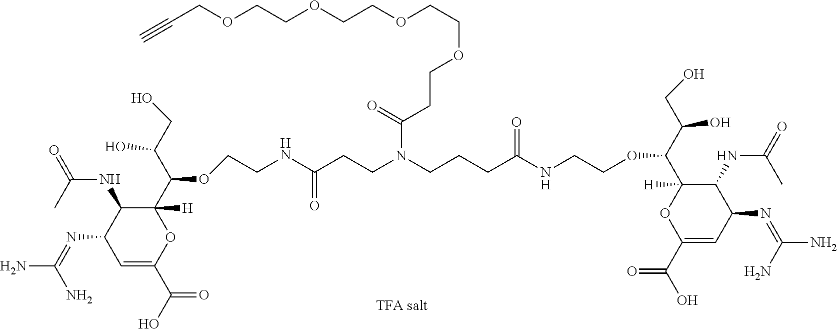 [SMILES: C#CCOCCOCCOCCOCCC(=O)N(CCCC(=O)NCCO[C@@H]([C@@H]1OC(C(=O)O)=C[C@H](N=C(N)N)[C@H]1NC(C)=O)[C@H](O)CO)CCC(=O)NCCO[C@@H]([C@@H]1OC(C(=O)O)=C[C@H](N=C(N)N)[C@H]1NC(C)=O)[C@H](O)CO] |
| Int-81 | 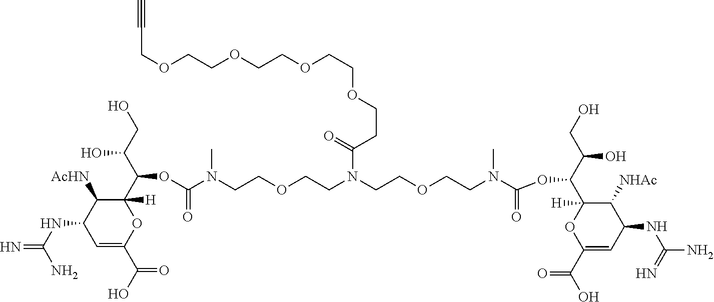 [SMILES: C#CCOCCOCCOCCOCCC(=O)N(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-82 | 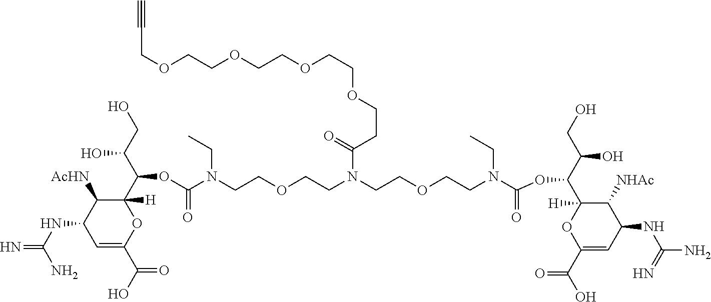 [SMILES: C#CCOCCOCCOCCOCCC(=O)N(CCOCCN(CC)C(=O)O[C@@H]([C@@H]1OC(C(=O)O)=C[C@H](NC(=N)N)[C@H]1NC(C)=O)[C@H](O)CO)CCOCCN(CC)C(=O)O[C@@H]([C@@H]1OC(C(=O)O)=C[C@H](NC(=N)N)[C@H]1NC(C)=O)[C@H](O)CO] |
| Int-83 | 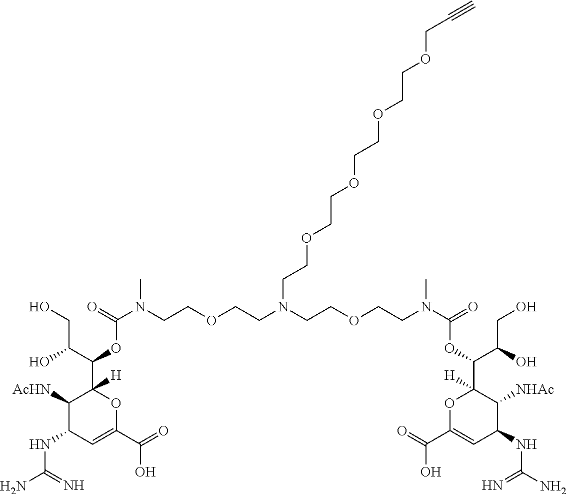 [SMILES: C#CCOCCOCCOCCOCCN(CCOCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-84 | 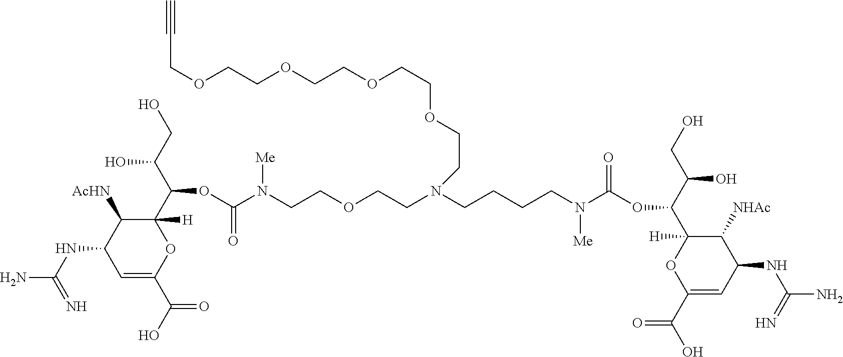 [SMILES: C#CCOCCOCCOCCOCCN(CCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-85 | 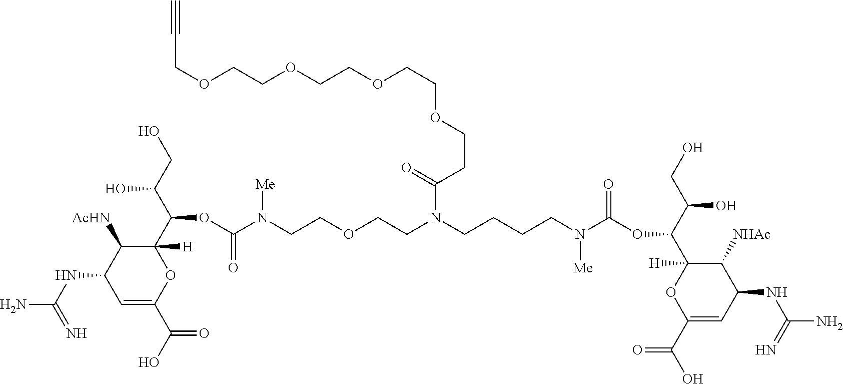 [SMILES: C#CCOCCOCCOCCOCCC(=O)N(CCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-86 | 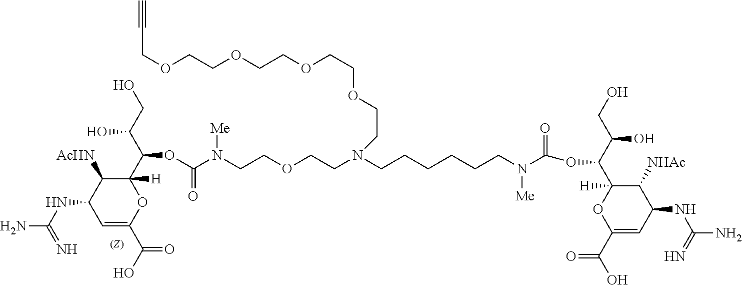 [SMILES: C#CCOCCOCCOCCOCCN(CCCCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-87 | 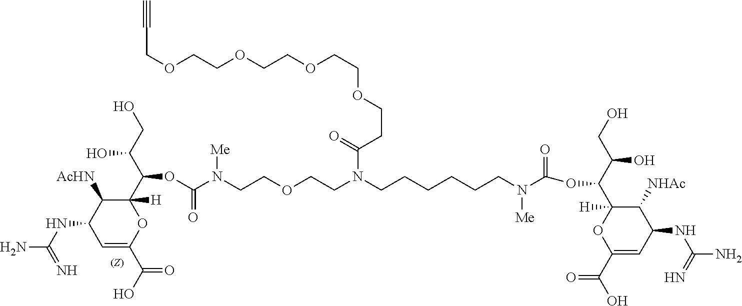 [SMILES: C#CCOCCOCCOCCOCCC(=O)N(CCCCCCN(C)C(=O)O[C@@H]([C@@H]1OC(C(=O)O)=C[C@H](NC(=N)N)[C@H]1NC(C)=O)[C@H](O)CO)CCOCCN(C)C(=O)O[C@@H]([C@@H]1OC(C(=O)O)=C[C@H](NC(=N)N)[C@H]1NC(C)=O)[C@H](O)CO] |
| Int-88 | 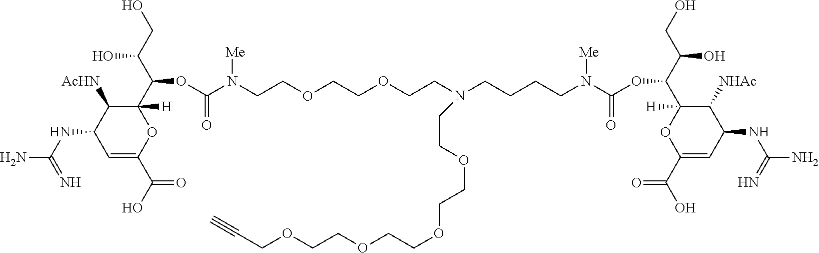 [SMILES: C#CCOCCOCCOCCOCCN(CCCCN(C)C(=O)O[C@@H]([C@@H]1OC(C(=O)O)=C[C@H](NC(=N)N)[C@H]1NC(C)=O)[C@H](O)CO)CCOCCOCCN(C)C(=O)O[C@@H]([C@@H]1OC(C(=O)O)=C[C@H](NC(=N)N)[C@H]1NC(C)=O)[C@H](O)CO] |
| Int-89 | 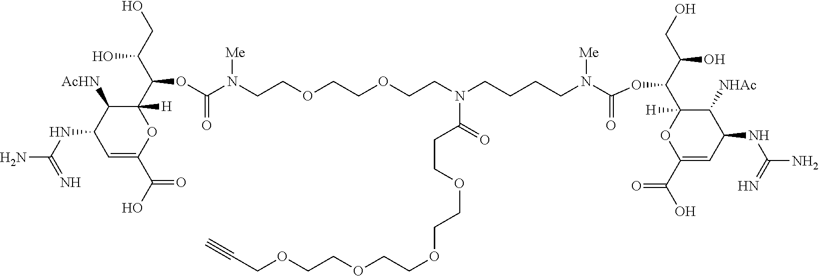 [SMILES: C#CCOCCOCCOCCOCCC(=O)N(CCCCN(C)C(=O)O[C@@H]([C@@H]1OC(C(=O)O)=C[C@H](NC(=N)N)[C@H]1NC(C)=O)[C@H](O)CO)CCOCCOCCN(C)C(=O)O[C@@H]([C@@H]1OC(C(=O)O)=C[C@H](NC(=N)N)[C@H]1NC(C)=O)[C@H](O)CO] |
| Int-90 | 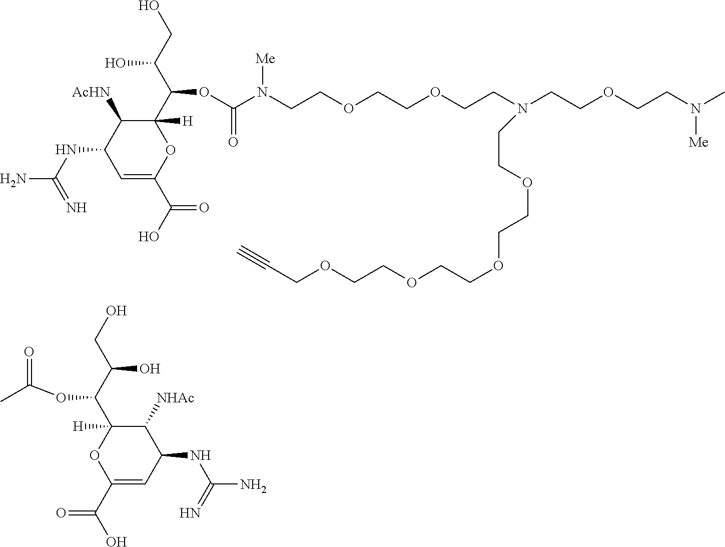 |
| Int-93 | 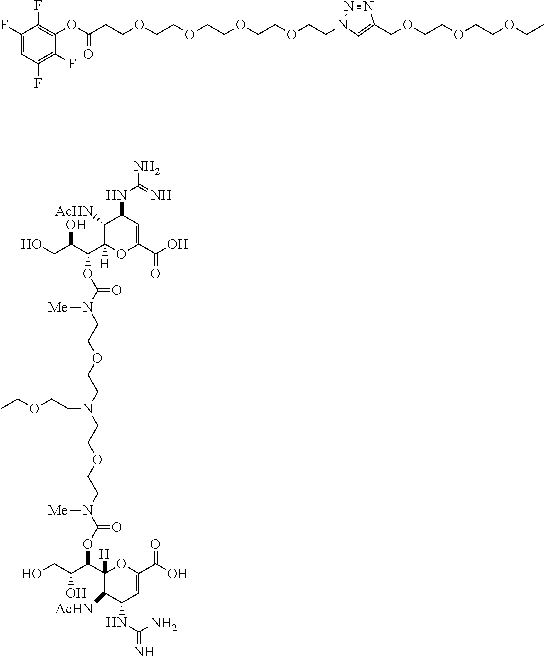 |
| Int-94 | 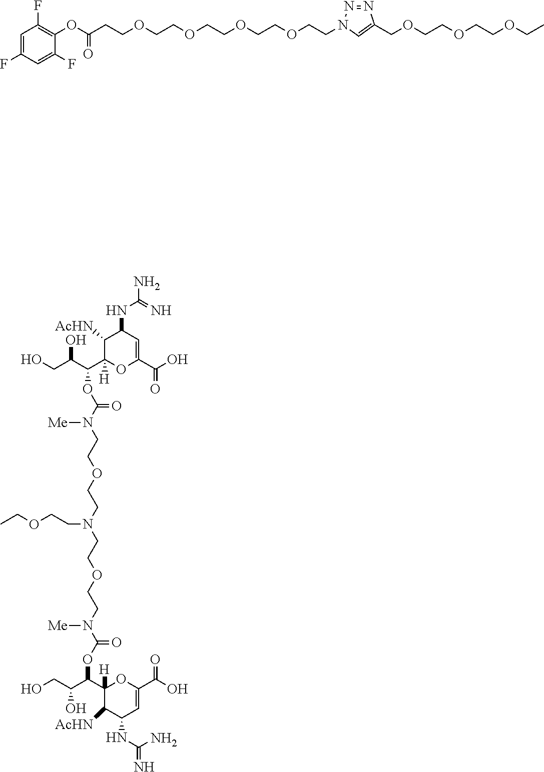 |

In some embodiments, E is an Fc domain monomer. In some embodiments, n is 2 and each E dimerizes to form an Fc domain. In some embodiments, the Fc domain monomer is human IgG1 or human IgG2, or a variant (e.g., mutational variant comprising between 1 and 10 amino acid substitutions) thereof.

In some embodiments, the squiggly line connected to E indicates that the L of each A₁-L or A₁-L-A₂ is covalently attached to a nitrogen atom of a solvent-exposed lysine of E.

In some embodiments, the squiggly line connected to E indicates that the L of each A₁-L or A₁-L-A₂ L is covalently attached to a sulfur atom of a solvent-exposed cysteine of E.

In some embodiments of any of the aspects described herein, the squiggly line of any one of formulas (M-I) or (D-I)-(D-VIII) may represent a covalent bond between E and the L of A₁-L or A₂-L-A₁. In some embodiments of any of the aspects described herein, the squiggly line of any one of formulas (M-I) or (D-I)-(D-VIII) may represent that one or more amino acid side chains of E (e.g., one or more nitrogen atoms of one or more surface exposed lysine residues of E or one or more sulfur atoms of one or more surface exposed cysteines in E) have been conjugated to a linker (e.g., a PEG₂-PEG₂₀ linker) wherein the linker has been functionalized with a reactive moiety, such that the reactive moiety forms a covalent bond with the L of any A₁-L or any A₂-L-A₁ described herein.

In some embodiments, T is an integer from 1 to 20 (e.g., 1, 2, 3, 4, 5, 6, 7, 8, 9, 10, 11, 12, 13, 14, 15, 16, 17, 18, 19, or 20).

In another aspect, the disclosure provides a population of conjugates having the structure of any of the conjugates described herein (e.g., a population of conjugates having the formula of any one of formulas (M-I) or (D-I)-(D-VIII)), wherein the average value of T is 1 to 20 (e.g., the average value of T is 1 to 2, 1 to 3, 1 to 4, 1 to 5, 5 to 10, 10 to 15, 15 to 20, 1.5 to 3.5, 2.5 to 4.5, 3.5 to 5.5, 4.5 to 6.5, 5.5 to 7.5, 6.5 to 8.5, 7.5 to 9.5, or 8.5 to 10.5).

In some embodiments of any of the aspects described herein, A₁ and/or A₂ have the structure described by (A-I):

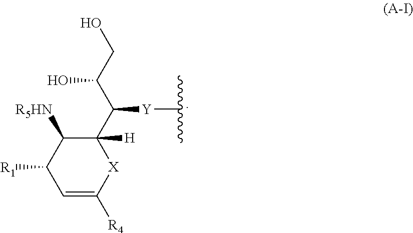

In preferred embodiments, wherein A₁ and/or A₂ have the structure described by (A-I): R₁ is —NHC(═NH)NH₂, R₄ is —CO₂H, R₅ is —COCH₃, and/or X is —O—. In preferred embodiments, A₁ and/or A₂ have the structure of zanamivir described by:

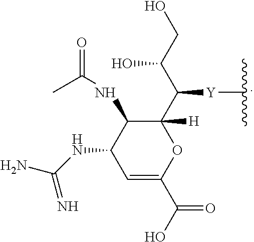

In preferred embodiments, A₁ and/or A₂ have the structure of zanamivir, where Y is —O(C═O)NCH₃—, described by:

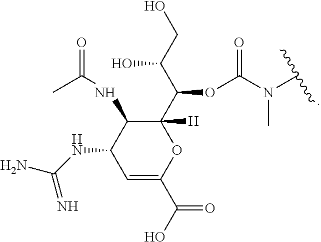 [SMILES: *N(C)C(=O)O[C@@H]([C@@H]1OC(C(=O)O)=C[C@H](NC(=N)N)[C@H]1NC(C)=O)[C@H](O)CO]

In some embodiments of any of the aspects described herein, A₁ and/or A₂ have the structure described by (A-II):

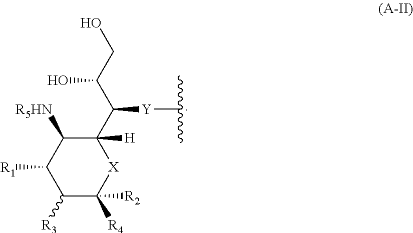

In preferred embodiments, wherein A₁ and/or A₂ have the structure described by (A-II): R₁ is —NHC(═NH)NH₂, R₂ is H or F, R₃ is H or F, R₄ is —CO₂H, R₅ is —COCH₃, and/or X is —O—. In preferred embodiments, A₁ and/or A₂ have the structure described by:

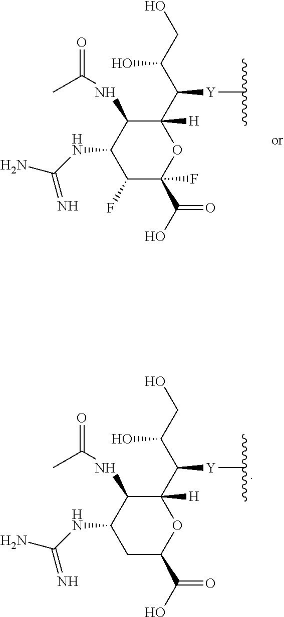

In some embodiments of any of the aspects described herein, A₁ and/or A₂ have the structure described by (A-III):

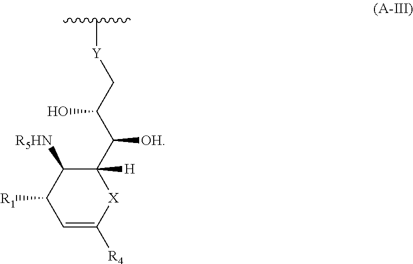

In preferred embodiments, wherein A₁ and/or A₂ have the structure described by (A-III): R₁ is —NHC(═NH)NH₂, R₄ is —CO₂H, R₅ is —COCH₃, and/or X is —O—. In preferred embodiments, A₁ and/or A₂ have the structure of zanamivir described by:

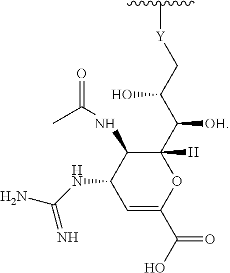

In some embodiments of any of the aspects described herein, A₁ and/or A₂ have the structure described by (A-IV):

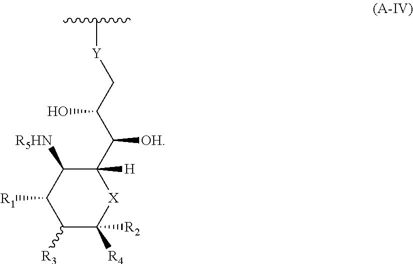

In preferred embodiments, wherein A₁ and/or A₂ have the structure described by (A-IV): R₁ is —NHC(═NH)NH₂, R₂ is H or F, R₃ is H or F, R₄ is —CO₂H, R₅ is —COCH₃, and/or X is —O—. In preferred embodiments, A₁ and/or A₂ have the structure described by:

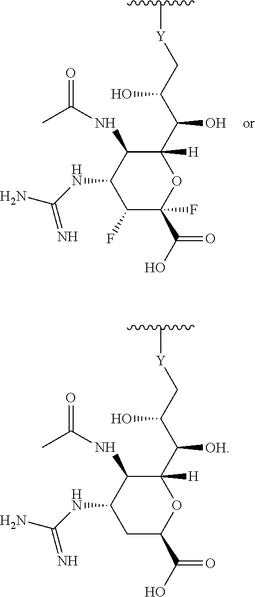

In some embodiments of any of the aspects described herein, A₁ and/or A₂ have the structure described by (A-V):

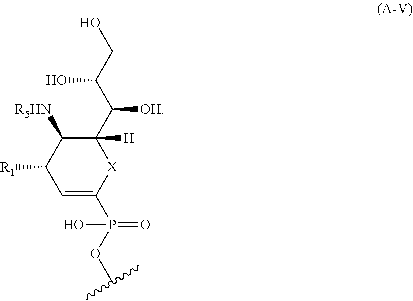

In preferred embodiments, wherein A₁ and/or A₂ have the structure described by (A-V): R₁ is —NHC(═NH)NH₂, R₅ is —COCH₃, and/or X is —O—. In preferred embodiments, A₁ and/or A₂ have the structure described by:

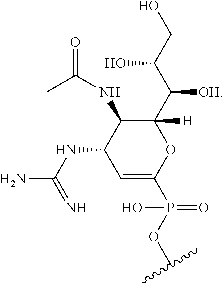 [SMILES: *OP(=O)(O)C1=C[C@H](NC(=N)N)[C@@H](NC(C)=O)[C@H]([C@H](O)[C@H](O)CO)O1]

In some embodiments of any of the aspects described herein, A₁ and/or A₂ have the structure described by (A-VI):

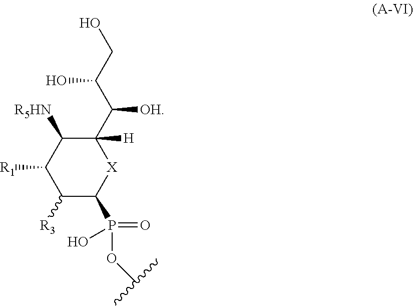

In preferred embodiments, wherein A₁ and/or A₂ have the structure described by (A-VI): R₁ is —NHC(═NH)NH₂, R₂ is H or F, R₃ is H or F, R₅ is —COCH₃, and/or X is —O—. In preferred embodiments, A₁ and/or A₂ have the structure described by:

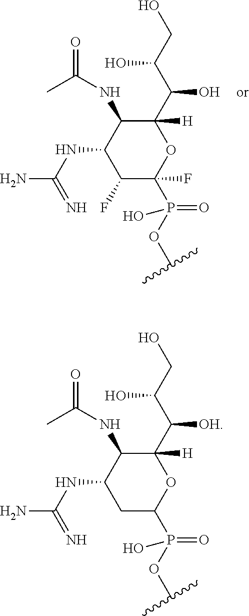 [SMILES: *OP(=O)(O)[C@]1(F)O[C@@H]([C@H](O)[C@H](O)CO)[C@H](NC(C)=O)[C@@H](NC(=N)N)[C@H]1F ; *OP(=O)(O)C1C[C@H](NC(=N)N)[C@@H](NC(C)=O)[C@H]([C@H](O)[C@H](O)CO)O1]

In some embodiments of any of the aspects described herein, A₁ and/or A₂ have the structure described by (A-VII):

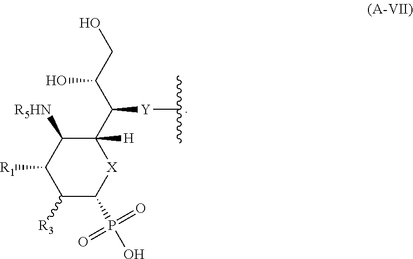

In preferred embodiments, wherein A₁ and/or A₂ have the structure described by (A-VII): R₁ is —NHC(═NH)NH₂, R₃ is H, R₅ is —COCH₃, and/or X is —O—. In preferred embodiments, A₁ and/or A₂ have the structure of sulfozanamivir described by:

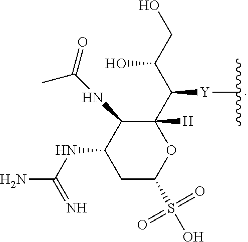

In some embodiments of any of the aspects described herein, A₁ and/or A₂ have the structure described by (A-VIII):

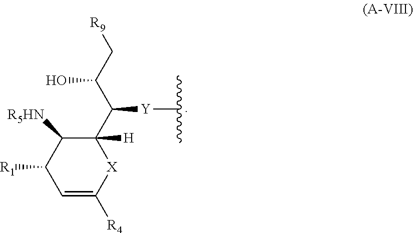

In some embodiments, each A₁ and each A₂ is described by formula (A-VIII-1):

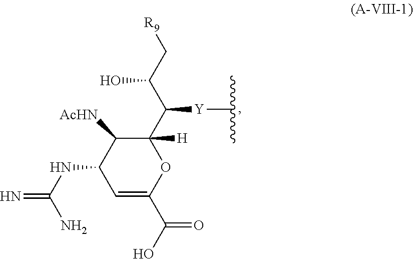

In some embodiments, each A₁ and each A₂ is independently selected from any one of formulas (A-VIII-1a)-(A-VIII-1d):

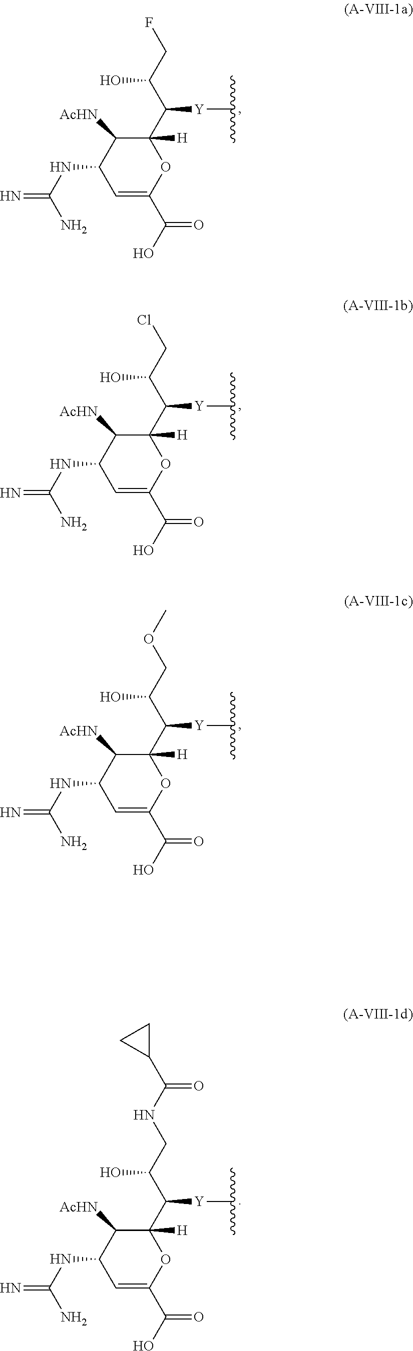

### Definitions

To facilitate the understanding of this disclosure, a number of terms are defined below. Terms defined herein have meanings as commonly understood by a person of ordinary skill in the areas relevant to the present disclosure. Terms such as “a”, “an,” and “the” are not intended to refer to only a singular entity, but include the general class of which a specific example may be used for illustration. The terminology herein is used to describe specific embodiments of the disclosure, but their usage does not delimit the disclosure, except as outlined in the claims.

The term “anti-influenza moiety,” as used herein, refers to a compound or composition that inhibits a function of the influenza virus. For example, and anti-influenza moiety may inhibit viral entry into a host cell or inhibit viral replication or proliferation. An anti-influenza moiety may target the virus or the host cell (e.g., a viral protein or a receptor on the host cell). In some embodiments, the anti-influenza moiety is a small molecule. Small molecule inhibitors of influenza (e.g., inhibitors of host cell entry or viral replication of influenza) are known in the art and include, for example, pimovidir, oseltamivir, zanamivir, sulfozanamivir, peramivir, laninamivir, amantadine, rimantadine, baloxavir (e.g., baloxavir acid or baloxavir marboxil), and chemical analogs thereof. Methods for measuring anti-viral activity are known in the art. An anti-influenza moiety may inhibit influenza A, B, C, parainfluenza, or any combination thereof.

The term “small molecule,” as used herein, refers to a low molecular weight compound (e.g., a compound (e.g., an organic compound) having less than 900 Da, that may regulate a biological process, with a size on the order of 1 nm. In some instances, a therapeutic agent is a small molecule therapeutic agent. In some instances, the small molecule agent is between about 300 and about 700 Da (e.g., about 325 Da, about 350 Da, about 375 Da, about 400 Da, about 425 Da, about 450 Da, about 475 Da, about 500 Da, about 525 Da, about 550 Da, about 575 Da, about 600 Da, about 625 Da, about 650 Da, or about 675 Da).

The term “neuraminidase inhibitor” or “viral neuraminidase inhibitor,” as used herein, refers to compounds that decreases the activity of the enzyme influenza virus neuraminidase (e.g., from influenza virus A, B, or C). A neuraminidase inhibitor may be identified by methods known to those of skill in the art, for example, by reduction of viral replication in an influenza viral plaque reduction assay, e.g., at concentrations less than 20 μM (e.g., less than 10 μM, 5 μM, 2 μM, 1 μM, 500 nM or 100 nM). Viral neuraminidase inhibitors known to those of skill in the art include zanamivir, sulfozanamivir, and analogs thereof (see, for example, Hadházi et al. A sulfozanamivir analogue has potent anti-influenza virus activity. Chem Med Chem Comm. 13:785-789 (2018)). In particular, zanamivir and analogs thereof include viral neuraminidase inhibitors of formulas (A-I)-(A-XIII).

The term “inhibits neuraminidase activity,” as used herein refers to an IC₅₀ of less than or equal to 1,000 nM. Specifically, the IC₅₀ represents the concentration of the influenza virus neuraminidase inhibitor that is required for 50% inhibition in vitro. In some aspects, an IC₅₀ of less than or equal to 100 nM or less than or equal to 10 nM in accordance with neuraminidase inhibition assay is indicative of a compound inhibiting neuraminidase activity.

By “viral infection” is meant the pathogenic growth of a virus (e.g., the influenza virus) in a host organism (e.g., a human subject). A viral infection can be any situation in which the presence of a viral population(s) is damaging to a host body. Thus, a subject is “suffering” from a viral infection when an excessive amount of a viral population is present in or on the subject's body, or when the presence of a viral population(s) is damaging the cells or other tissue of the subject.

As used herein, the term “Fc domain monomer” refers to a polypeptide chain that includes at least a hinge domain and second and third antibody constant domains (C_{H}2 and C_{H}3) or functional fragments thereof (e.g., fragments that that capable of (i) dimerizing with another Fc domain monomer to form an Fc domain, and (ii) binding to an Fc receptor. The Fc domain monomer can be any immunoglobulin antibody isotype, including IgG, IgE, IgM, IgA, or IgD (e.g., IgG). Additionally, the Fc domain monomer can be an IgG subtype (e.g., IgG1, IgG2a, IgG2b, IgG3, or IgG4) (e.g., IgG1). An Fc domain monomer does not include any portion of an immunoglobulin that is capable of acting as an antigen-recognition region, e.g., a variable domain or a complementarity determining region (CDR). Fc domain monomers in the conjugates as described herein can contain one or more changes from a wild-type Fc domain monomer sequence (e.g., 1-10, 1-8, 1-6, 1-4 amino acid substitutions, additions, or deletions) that alter the interaction between an Fc domain and an Fc receptor. Examples of suitable changes are known in the art. In some embodiments, the N-terminus of the variant Fc domain monomer is any one of amino acid residues 198-205. In some embodiments, the N-terminus of the variant Fc domain monomer is amino acid residue 201 (e.g., Asn 201). In some embodiments, the N-terminus of the variant Fc domain monomer is amino acid residue 202 (e.g., Val 202). In some embodiments, the C-terminus of the variant Fc domain monomer is any one of amino acid residues 437-447. In some embodiments, the C-terminus of the variant Fc domain monomer is amino acid residue 446 (e.g., Gly 446). In some embodiments, the C-terminus of the variant Fc domain monomer is amino acid residue 447 (e.g. Lys 447). C-terminal Lys447 of the Fc region may or may not be present, without affecting the structure or stability of the Fc region. C-terminal Lys 447 may be proteolytically cleaved upon expression of the polypeptide. Unless otherwise specified herein, numbering of amino acid residues in the IgG or Fc domain monomer is according to the EU numbering system for antibodies, also called the Kabat EU index, as described, for example, in Kabat et al., Sequences of Proteins of Immunological Interest, 5th Ed. Public Health Service, National Institutes of Health, Bethesda, MD, 1991.

As used herein, the term “Fc domain” refers to a dimer of two Fc domain monomers that is capable of binding an Fc receptor. In the wild-type Fc domain, the two Fc domain monomers dimerize by the interaction between the two C_{H}3 antibody constant domains, in some embodiments, one or more disulfide bonds form between the hinge domains of the two dimerizing Fc domain monomers. The term “covalently attached” refers to two parts of a conjugate that are linked to each other by a covalent bond formed between two atoms in the two parts of the conjugate.

As used-herein, a “surface exposed amino acid” or “solvent-exposed amino acid,” such as a surface exposed cysteine or a surface exposed lysine refers to an amino acid that is accessible to the solvent surrounding the protein. A surface exposed amino acid may be a naturally-occurring or an engineered variant (e.g., a substitution or insertion) of the protein. In some embodiments, a surface exposed amino acid is an amino acid that when substituted does not substantially change the three-dimensional structure of the protein.

The terms “linker,” “L,” and “L′,” as used herein, refer to a covalent linkage or connection between two or more components in a conjugate (e.g., between two neuraminidase inhibitors in a conjugate described herein, between a anti-influenza moiety (e.g., neuraminidase inhibitor) and an Fc domain in a conjugate described herein, and between a dimer of two neuraminidase inhibitors and an Fc domain in a conjugate described herein). In some embodiments, a conjugate described herein may contain a linker that has a trivalent structure (e.g., a trivalent linker). A trivalent linker has three arms, in which each arm is covalently linked to a component of the conjugate (e.g., a first arm conjugated to a first neuraminidase inhibitor, a second arm conjugated to a second neuraminidase inhibitor, and a third arm conjugated to an Fc domain).

Molecules that may be used as linkers include at least two functional groups, which may be the same or different, e.g., two carboxylic acid groups, two amine groups, two sulfonic acid groups, a carboxylic acid group and a maleimide group, a carboxylic acid group and an alkyne group, a carboxylic acid group and an amine group, a carboxylic acid group and a sulfonic acid group, an amine group and a maleimide group, an amine group and an alkyne group, or an amine group and a sulfonic acid group. The first functional group may form a covalent linkage with a first component in the conjugate and the second functional group may form a covalent linkage with the second component in the conjugate. In some embodiments of a trivalent linker, two arms of a linker may contain two dicarboxylic acids, in which the first carboxylic acid may form a covalent linkage with the first anti-influenza moiety (e.g., neuraminidase inhibitor) in the conjugate and the second carboxylic acid may form a covalent linkage with the second anti-influenza moiety (e.g., neuraminidase inhibitor) in the conjugate, and the third arm of the linker may for a covalent linkage with an Fc domain in the conjugate. Examples of dicarboxylic acids are described further herein. In some embodiments, a molecule containing one or more maleimide groups may be used as a linker, in which the maleimide group may form a carbon-sulfur linkage with a cysteine in a component (e.g., an Fc domain) in the conjugate. In some embodiments, a molecule containing one or more alkyne groups may be used as a linker, in which the alkyne group may form a 1,2,3-triazole linkage with an azide in a component (e.g., an Fc domain) in the conjugate. In some embodiments, a molecule containing one or more azide groups may be used as a linker, in which the azide group may form a 1,2,3-triazole linkage with an alkyne in a component (e.g., an Fc domain) in the conjugate. In some embodiments, a molecule containing one or more bis-sulfone groups may be used as a linker, in which the bis-sulfone group may form a linkage with an amine group a component (e.g., an Fc domain) in the conjugate. In some embodiments, a molecule containing one or more sulfonic acid groups may be used as a linker, in which the sulfonic acid group may form a sulfonamide linkage with a component in the conjugate. In some embodiments, a molecule containing one or more isocyanate groups may be used as a linker, in which the isocyanate group may form a urea linkage with a component in the conjugate. In some embodiments, a molecule containing one or more haloalkyl groups may be used as a linker, in which the haloalkyl group may form a covalent linkage, e.g., C—N and C—O linkages, with a component in the conjugate.

In some embodiments, a linker provides space, rigidity, and/or flexibility between the two or more components. In some embodiments, a linker may be a bond, e.g., a covalent bond. The term “bond” refers to a chemical bond, e.g., an amide bond, a disulfide bond, a C—O bond, a C—N bond, a N—N bond, a C—S bond, or any kind of bond created from a chemical reaction, e.g., chemical conjugation. In some embodiments, a linker includes no more than 250 atoms. In some embodiments, a linker includes no more than 250 non-hydrogen atoms. In some embodiments, the backbone of a linker includes no more than 250 atoms. The “backbone” of a linker refers to the atoms in the linker that together form the shortest path from one part of a conjugate to another part of the conjugate (e.g., the shortest path linking a first anti-influenza moiety (e.g., neuraminidase inhibitor) and a second neuraminidase inhibitor). The atoms in the backbone of the linker are directly involved in linking one part of a conjugate to another part of the conjugate (e.g., linking a first anti-influenza moiety (e.g., neuraminidase inhibitor) and a second neuraminidase inhibitor). For examples, hydrogen atoms attached to carbons in the backbone of the linker are not considered as directly involved in linking one part of the conjugate to another part of the conjugate.

In some embodiments, a linker may comprise a synthetic group derived from, e.g., a synthetic polymer (e.g., a polyethylene glycol (PEG) polymer). In some embodiments, a linker may comprise one or more amino acid residues, such as D- or L-amino acid residues. In some embodiments, a linker may be a residue of an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker may comprise one or more, e.g., 1-100, 1-50, 1-25, 1-10, 1-5, or 1-3, optionally substituted alkylene, optionally substituted heteroalkylene (e.g., a PEG unit), optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted cycloalkenylene, optionally substituted heterocycloalkenylene, optionally substituted cycloalkynylene, optionally substituted heterocycloalkynylene, optionally substituted arylene, optionally substituted heteroarylene (e.g., pyridine), O, S, NRⁱ (Rⁱ is H, optionally substituted alkyl, optionally substituted heteroalkyl, optionally substituted alkenyl, optionally substituted heteroalkenyl, optionally substituted alkynyl, optionally substituted heteroalkynyl, optionally substituted cycloalkyl, optionally substituted heterocycloalkyl, optionally substituted cycloalkenyl, optionally substituted heterocycloalkenyl, optionally substituted cycloalkynyl, optionally substituted heterocycloalkynyl, optionally substituted aryl, or optionally substituted heteroaryl), P, carbonyl, thiocarbonyl, sulfonyl, phosphate, phosphoryl, or imino. For example, a linker may comprise one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

The terms “alkyl,” “alkenyl,” and “alkynyl,” as used herein, include straight-chain and branched-chain monovalent substituents, as well as combinations of these, containing only C and H when unsubstituted. When the alkyl group includes at least one carbon-carbon double bond or carbon-carbon triple bond, the alkyl group can be referred to as an “alkenyl” or “alkynyl” group respectively. The monovalency of an alkyl, alkenyl, or alkynyl group does not include the optional substituents on the alkyl, alkenyl, or alkynyl group. For example, if an alkyl, alkenyl, or alkynyl group is attached to a compound, monovalency of the alkyl, alkenyl, or alkynyl group refers to its attachment to the compound and does not include any additional substituents that may be present on the alkyl, alkenyl, or alkynyl group. In some embodiments, the alkyl or heteroalkyl group may contain, e.g., 1-20. 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, the alkenyl, heteroalkenyl, alkynyl, or heteroalkynyl group may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12, C2-C10, C2-C8, C2-C6, or C2-C4). Examples include, but are not limited to, methyl, ethyl, isobutyl, sec-butyl, tert-butyl, 2-propenyl, and 3-butynyl.

The term “cycloalkyl,” as used herein, represents a monovalent saturated or unsaturated non-aromatic cyclic alkyl group. A cycloalkyl may have, e.g., three to twenty carbons (e.g., a C3-C7, C3-C8, C3-C9, C3-C10, C3-C11, C3-C12, C3-C14, C3-C16, C3-C18, or C3-C20 cycloalkyl). Examples of cycloalkyls include, but are not limited to, cyclopropyl, cyclobutyl, cyclopentyl, cyclohexyl, and cycloheptyl. When the cycloalkyl group includes at least one carbon-carbon double bond, the cycloalkyl group can be referred to as a “cycloalkenyl” group. A cycloalkenyl may have, e.g., four to twenty carbons (e.g., a C4-C7, C4-C8, C4-C9, C4-C10, C4-C11, C4-C12, C4-C14, C4-C16, C4-C18, or C4-C20 cycloalkenyl). Exemplary cycloalkenyl groups include, but are not limited to, cyclopentenyl, cyclohexenyl, and cycloheptenyl. When the cycloalkyl group includes at least one carbon-carbon triple bond, the cycloalkyl group can be referred to as a “cycloalkynyl” group. A cycloalkynyl may have, e.g., eight to twenty carbons (e.g., a C8-C9, C8-C10, C8-C11, C8-C12, C8-C14, C8-C16, C8-C18, or C8-C20 cycloalkynyl). The term “cycloalkyl” also includes a cyclic compound having a bridged multicyclic structure in which one or more carbons bridges two non-adjacent members of a monocyclic ring, e.g., bicyclo[2.2.1.]heptyl and adamantane. The term “cycloalkyl” also includes bicyclic, tricyclic, and tetracyclic fused ring structures, e.g., decalin and spiro cyclic compounds.

The term “aryl,” as used herein, refers to any monocyclic or fused ring bicyclic or tricyclic system which has the characteristics of aromaticity in terms of electron distribution throughout the ring system, e.g., phenyl, naphthyl, or phenanthrene. In some embodiments, a ring system contains 5-15 ring member atoms or 5-10 ring member atoms. An aryl group may have, e.g., five to fifteen carbons (e.g., a C5-C6, C5-C7, C5-C8, C5-C9, C5-C10, C5-C11, C5-C12, C5-C13, C5-C14, or C5-C15 aryl). The term “heteroaryl” also refers to such monocyclic or fused bicyclic ring systems containing one or more, e.g., 1-4, 1-3, 1, 2, 3, or 4, heteroatoms selected from O, S and N. A heteroaryl group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroaryl). The inclusion of a heteroatom permits inclusion of 5-membered rings to be considered aromatic as well as 6-membered rings. Thus, typical heteroaryl systems include, e.g., pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, isoxazolyl, benzoxazolyl, benzoisoxazolyl, and imidazolyl. Because tautomers are possible, a group such as phthalimido is also considered heteroaryl. In some embodiments, the aryl or heteroaryl group is a 5- or 6-membered aromatic rings system optionally containing 1-2 nitrogen atoms. In some embodiments, the aryl or heteroaryl group is an optionally substituted phenyl, pyridyl, indolyl, pyrimidyl, pyridazinyl, benzothiazolyl, benzimidazolyl, pyrazolyl, imidazolyl, isoxazolyl, thiazolyl, or imidazopyridinyl. In some embodiments, the aryl group is phenyl. In some embodiments, an aryl group may be optionally substituted with a substituent such an aryl substituent, e.g., biphenyl.

The term “alkaryl,” refers to an aryl group that is connected to an alkylene, alkenylene, or alkynylene group. In general, if a compound is attached to an alkaryl group, the alkylene, alkenylene, or alkynylene portion of the alkaryl is attached to the compound. In some embodiments, an alkaryl is C6-C35 alkaryl (e.g., C6-C16, C6-C14, C6-C12, C6-C10, C6-C9, C6-C8, C7, or C6 alkaryl), in which the number of carbons indicates the total number of carbons in both the aryl portion and the alkylene, alkenylene, or alkynylene portion of the alkaryl. Examples of alkaryls include, but are not limited to, (C1-C8)alkylene(C6-C12)aryl, (C2-C8)alkenylene(C6-C12)aryl, or (C2-C8)alkynylene(C6-C12)aryl. In some embodiments, an alkaryl is benzyl or phenethyl. In a heteroalkaryl, one or more heteroatoms selected from N, O, and S may be present in the alkylene, alkenylene, or alkynylene portion of the alkaryl group and/or may be present in the aryl portion of the alkaryl group. In an optionally substituted alkaryl, the substituent may be present on the alkylene, alkenylene, or alkynylene portion of the alkaryl group and/or may be present on the aryl portion of the alkaryl group.

The term “amino,” as used herein, represents —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino group is —NH₂.

The term “alkamino,” as used herein, refers to an amino group, described herein, that is attached to an alkylene (e.g., C1-C5 alkylene), alkenylene (e.g., C2-C5 alkenylene), or alkynylene group (e.g., C2-C5 alkenylene). In general, if a compound is attached to an alkamino group, the alkylene, alkenylene, or alkynylene portion of the alkamino is attached to the compound. The amino portion of an alkamino refers to —N(R^{x})₂ or —N⁺(R^{x})₃, where each R^{x} is, independently, H, alkyl, alkenyl, alkynyl, aryl, alkaryl, cycloalkyl, or two R^{x} combine to form a heterocycloalkyl. In some embodiment, the amino portion of an alkamino is —NH₂. An example of an alkamino group is C1-C5 alkamino, e.g., C2 alkamino (e.g., CH₂CH₂NH₂ or CH₂CH₂N(CH₃)₂). In a heteroalkamino group, one or more, e.g., 1-4, 1-3, 1, 2, 3, or 4, heteroatoms selected from N, O, and S may be present in the alkylene, alkenylene, or alkynylene portion of the heteroalkamino group. In some embodiments, an alkamino group may be optionally substituted. In a substituted alkamino group, the substituent may be present on the alkylene, alkenylene, or alkynylene portion of the alkamino group and/or may be present on the amino portion of the alkamino group. The term “alkamide,” as used herein, refers to an amide group that is attached to an alkylene (e.g., C1-C5 alkylene), alkenylene (e.g., C2-C5 alkenylene), or alkynylene (e.g., C2-C5 alkenylene) group. In general, if a compound is attached to an alkamide group, the alkylene, alkenylene, or alkynylene portion of the alkamide is attached to the compound. The amide portion of an alkamide refers to —C(O)—N(R^{x})₂, where each R^{x} is, independently, H, alkyl, alkenyl, alkynyl, aryl, alkaryl, cycloalkyl, or two R^{x} combine to form a heterocycloalkyl. In some embodiment, the amide portion of an alkamide is —C(O)NH₂. An alkamide group may be —(CH₂)₂—C(O)NH₂ or —CH₂—C(O)NH₂. In a heteroalkamide group, one or more, e.g., 1-4, 1-3, 1, 2, 3, or 4, heteroatoms selected from N, O, and S may be present in the alkylene, alkenylene, or alkynylene portion of the heteroalkamide group. In some embodiments, an alkamide group may be optionally substituted. In a substituted alkamide group, the substituent may be present on the alkylene, alkenylene, or alkynylene portion of the alkamide group and/or may be present on the amide portion of the alkamide group.

The terms “alkylene,” “alkenylene,” and “alkynylene,” as used herein, refer to divalent groups having a specified size. In some embodiments, an alkylene may contain, e.g., 1-20, 1-18, 1-16, 1-14, 1-12, 1-10, 1-8, 1-6, 1-4, or 1-2 carbon atoms (e.g., C1-C20, C1-C18, C1-C16, C1-C14, C1-C12, C1-C10, C1-C8, C1-C6, C1-C4, or C1-C2). In some embodiments, an alkenylene or alkynylene may contain, e.g., 2-20, 2-18, 2-16, 2-14, 2-12, 2-10, 2-8, 2-6, or 2-4 carbon atoms (e.g., C2-C20, C2-C18, C2-C16, C2-C14, C2-C12, C2-C10, C2-C8, C2-C6, or C2-C4). Alkylene, alkenylene, and/or alkynylene includes straight-chain and branched-chain forms, as well as combinations of these. The divalency of an alkylene, alkenylene, or alkynylene group does not include the optional substituents on the alkylene, alkenylene, or alkynylene group. For example, two neuraminidase inhibitors may be attached to each other by way of a linker that includes alkylene, alkenylene, and/or alkynylene, or combinations thereof. Each of the alkylene, alkenylene, and/or alkynylene groups in the linker is considered divalent with respect to the two attachments on either end of alkylene, alkenylene, and/or alkynylene group. For example, if a linker includes -(optionally substituted alkylene)-(optionally substituted alkenylene)-(optionally substituted alkylene)-, the alkenylene is considered divalent with respect to its attachments to the two alkylenes at the ends of the linker. The optional substituents on the alkenylene are not included in the divalency of the alkenylene. The divalent nature of an alkylene, alkenylene, or alkynylene group (e.g., an alkylene, alkenylene, or alkynylene group in a linker) refers to both of the ends of the group and does not include optional substituents that may be present in an alkylene, alkenylene, or alkynylene group. Because they are divalent, they can link together multiple (e.g., two) parts of a conjugate, e.g., a first anti-influenza moiety (e.g., neuraminidase inhibitor) and a second neuraminidase inhibitor. Alkylene, alkenylene, and/or alkynylene groups can be substituted by the groups typically suitable as substituents for alkyl, alkenyl and alkynyl groups as set forth herein. For example, C═O is a C1 alkylene that is substituted by an oxo (═O). For example, —HCR—C≡C— may be considered as an optionally substituted alkynylene and is considered a divalent group even though it has an optional substituent, R. Heteroalkylene, heteroalkenylene, and/or heteroalkynylene groups refer to alkylene, alkenylene, and/or alkynylene groups including one or more, e.g., 1-4, 1-3, 1, 2, 3, or 4, heteroatoms, e.g., N, O, and S. For example, a polyethylene glycol (PEG) polymer or a PEG unit —(CH₂)₂—O— in a PEG polymer is considered a heteroalkylene containing one or more oxygen atoms.

The term “cycloalkylene,” as used herein, refers to a divalent cyclic group linking together two parts of a compound. For example, one carbon within the cycloalkylene group may be linked to one part of the compound, while another carbon within the cycloalkylene group may be linked to another part of the compound. A cycloalkylene group may include saturated or unsaturated non-aromatic cyclic groups. A cycloalkylene may have, e.g., three to twenty carbons in the cyclic portion of the cycloalkylene (e.g., a C3-C7, C3-C8, C3-C9, C3-C10, C3-C11, C3-C12, C3-C14, C3-C16, C3-C18, or C3-C20 cycloalkylene). When the cycloalkylene group includes at least one carbon-carbon double bond, the cycloalkylene group can be referred to as a “cycloalkenylene” group. A cycloalkenylene may have, e.g., four to twenty carbons in the cyclic portion of the cycloalkenylene (e.g., a C4-C7, C4-C8, C4-C9. C4-C10, C4-C11, C4-C12, C4-C14, C4-C16, C4-C18, or C4-C20 cycloalkenylene). When the cycloalkylene group includes at least one carbon-carbon triple bond, the cycloalkylene group can be referred to as a “cycloalkynylene” group. A cycloalkynylene may have, e.g., four to twenty carbons in the cyclic portion of the cycloalkynylene (e.g., a C4-C7, C4-C8, C4-C9. C4-C10, C4-C11, C4-C12, C4-C14, C4-C16, C4-C18, or C8-C20 cycloalkynylene). A cycloalkylene group can be substituted by the groups typically suitable as substituents for alkyl, alkenyl and alkynyl groups as set forth herein. Heterocycloalkylene refers to a cycloalkylene group including one or more, e.g., 1-4, 1-3, 1, 2, 3, or 4, heteroatoms, e.g., N, O, and S. Examples of cycloalkylenes include, but are not limited to, cyclopropylene and cyclobutylene. A tetrahydrofuran may be considered as a heterocycloalkylene.

The term “arylene,” as used herein, refers to a multivalent (e.g., divalent or trivalent) aryl group linking together multiple (e.g., two or three) parts of a compound. For example, one carbon within the arylene group may be linked to one part of the compound, while another carbon within the arylene group may be linked to another part of the compound. An arylene may have, e.g., five to fifteen carbons in the aryl portion of the arylene (e.g., a C5-C6, C5-C7, C5-C8, C5-C9. C5-C10, C5-C11, C5-C12, C5-C13, C5-C14, or C5-C15 arylene). An arylene group can be substituted by the groups typically suitable as substituents for alkyl, alkenyl and alkynyl groups as set forth herein. Heteroarylene refers to an aromatic group including one or more, e.g., 1-4, 1-3, 1, 2, 3, or 4, heteroatoms, e.g., N, O, and S. A heteroarylene group may have, e.g., two to fifteen carbons (e.g., a C2-C3, C2-C4, C2-C5, C2-C6, C2-C7, C2-C8, C2-C9. C2-C10, C2-C11, C2-C12, C2-C13, C2-C14, or C2-C15 heteroarylene).

The term “optionally substituted,” as used herein, refers to having 0, 1, or more substituents, such as 0-25, 0-20, 0-10 or 0-5 substituents. Substituents include, but are not limited to, alkyl, alkenyl, alkynyl, aryl, alkaryl, acyl, heteroaryl, heteroalkyl, heteroalkenyl, heteroalkynyl, heteroalkaryl, halogen, oxo, cyano, nitro, amino, alkamino, hydroxy, alkoxy, alkanoyl, carbonyl, carbamoyl, guanidinyl, ureido, amidinyl, any of the groups or moieties described above, and hetero versions of any of the groups or moieties described above. Substituents include, but are not limited to, F, Cl, methyl, phenyl, benzyl, OR, NR₂, SR, SOR, SO₂R, OCOR, NRCOR, NRCONR₂, NRCOOR, OCONR₂, RCO, COOR, alkyl-OOCR, SO₃R, CONR₂, SO₂NR₂, NRSO₂NR₂, CN, CF₃, OCF₃, SiR₃, and NO₂, wherein each R is, independently, H, alkyl, alkenyl, aryl, heteroalkyl, heteroalkenyl, or heteroaryl, and wherein two of the optional substituents on the same or adjacent atoms can be joined to form a fused, optionally substituted aromatic or nonaromatic, saturated or unsaturated ring which contains 3-8 members, or two of the optional substituents on the same atom can be joined to form an optionally substituted aromatic or nonaromatic, saturated or unsaturated ring which contains 3-8 members.

An optionally substituted group or moiety refers to a group or moiety (e.g., any one of the groups or moieties described above) in which one of the atoms (e.g., a hydrogen atom) is optionally replaced with another substituent. For example, an optionally substituted alkyl may be an optionally substituted methyl, in which a hydrogen atom of the methyl group is replaced by, e.g., OH. As another example, a substituent on a heteroalkyl or its divalent counterpart, heteroalkylene, may replace a hydrogen on a carbon or a hydrogen on a heteroatom such as N. For example, the hydrogen atom in the group —R—NH—R— may be substituted with an alkamide substituent, e.g., —R—N[(CH₂C(O)N(CH₃)₂]—R. Generally, an optional substituent is a noninterfering substituent. A “noninterfering substituent” refers to a substituent that leaves the ability of the conjugates described herein (e.g., conjugates of any one of formulas (M-I) or (D-I)-(D-VIII)) to either bind to viral neuraminidase or to inhibit the proliferation of influenza virus. Thus, in some embodiments, the substituent may alter the degree of such activity. However, as long as the conjugate retains the ability to bind to viral neuraminidase or to inhibitor viral proliferation, the substituent will be classified as “noninterfering.” For example, the noninterfering substituent would leave the ability of the compound to provide antiviral efficacy based on an IC50 value of 10 μM or less in a viral plaque reduction assay. Thus, the substituent may alter the degree of inhibition based on plaque reduction or influenza virus neuraminidase inhibition. However, as long as the compounds herein such as compounds of formulas (A-I)-(A-XIII) retain the ability to inhibit influenza virus neuraminidase activity, the substituent will be classified as “noninterfering.” A number of assays for determining viral plaque reduction or the ability of any compound to inhibit influenza virus neuraminidase are available in the art, and some are exemplified in the Examples below.

The term “hetero,” when used to describe a chemical group or moiety, refers to having at least one heteroatom that is not a carbon or a hydrogen, e.g., N, O, and S. Any one of the groups or moieties described above may be referred to as hetero if it contains at least one heteroatom. For example, a heterocycloalkyl, heterocycloalkenyl, or heterocycloalkynyl group refers to a cycloalkyl, cycloalkenyl, or cycloalkynyl group that has one or more heteroatoms independently selected from, e.g., N, O, and S. An example of a heterocycloalkenyl group is a maleimido. For example, a heteroaryl group refers to an aromatic group that has one or more heteroatoms independently selected from, e.g., N, O, and S. One or more heteroatoms may also be included in a substituent that replaced a hydrogen atom in a group or moiety as described herein. For example, in an optionally substituted heteroaryl group, if one of the hydrogen atoms in the heteroaryl group is replaced with a substituent (e.g., methyl), the substituent may also contain one or more heteroatoms (e.g., methanol).

The term “acyl,” as used herein, refers to a group having the structure:

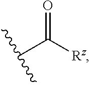

wherein R^{z} is an optionally substituted alkyl, alkenyl, alkynyl, cycloalkyl, cycloalkenyl, cycloalkynyl, aryl, alkaryl, alkamino, heteroalkyl, heteroalkenyl, heteroalkynyl, heterocycloalkyl, heterocycloalkenyl, heterocycloalkynyl, heteroaryl, heteroalkaryl, or heteroalkamino.

The term “halo” or “halogen,” as used herein, refers to any halogen atom, e.g., F, Cl, Br, or I. Any one of the groups or moieties described herein may be referred to as a “halo moiety” if it contains at least one halogen atom, such as haloalkyl.

The term “hydroxyl,” as used herein, represents an —OH group.

The term “oxo,” as used herein, refers to a substituent having the structure ═O, where there is a double bond between an atom and an oxygen atom.

The term “carbonyl,” as used herein, refers to a group having the structure:

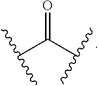 [SMILES: *C(*)=O]

The term “thiocarbonyl,” as used herein, refers to a group having the structure:

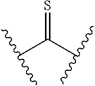 [SMILES: *C(*)=S]

The term “phosphate,” as used herein, represents the group having the structure:

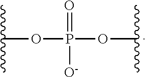 [SMILES: *OP(=O)([O-])O*]

The term “phosphoryl,” as used herein, represents the group having the structure:

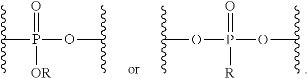

The term “sulfonyl,” as used herein, represents the group having the structure:

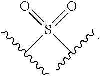 [SMILES: *S(*)(=O)=O]

The term “imino,” as used herein, represents the group having the structure:

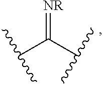

wherein R is an optional substituent.

The term “N-protecting group,” as used herein, represents those groups intended to protect an amino group against undesirable reactions during synthetic procedures. Commonly used N-protecting groups are disclosed in Greene, “Protective Groups in Organic Synthesis,” 5th Edition (John Wiley & Sons, New York, 2014), which is incorporated herein by reference. N-protecting groups include, e.g., acyl, aryloyl, and carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, carboxybenzyl (CBz), 4-chlorobenzoyl, 4-bromobenzoyl, 4-nitrobenzoyl, and chiral auxiliaries such as protected or unprotected D, L or D, L-amino acid residues such as alanine, leucine, phenylalanine; sulfonyl-containing groups such as benzenesulfonyl and p-toluenesulfonyl; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl (BOC), diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl (Fmoc), cyclopentyloxycarbonyl, adamantyloxycarbonyl, cyclohexyloxycarbonyl, and phenylthiocarbonyl; alkaryl groups such as benzyl, triphenylmethyl, and benzyloxymethyl; and silyl groups such as trimethylsilyl.

The term “amino acid,” as used herein, means naturally occurring amino acids and non-naturally occurring amino acids.

The term “naturally occurring amino acids,” as used herein, means amino acids including Ala, Arg, Asn, Asp, Cys, Gln, Glu, Gly, His, lie, Leu, Lys, Met, Phe, Pro, Ser, Thr, Trp, Tyr, and Val.

The term “non-naturally occurring amino acid,” as used herein, means an alpha amino acid that is not naturally produced or found in a mammal.

As used herein, the term “percent (%) identity” refers to the percentage of amino acid residues of a candidate sequence, e.g., an Fc-IgG, or fragment thereof, that are identical to the amino acid residues of a reference sequence after aligning the sequences and introducing gaps, if necessary, to achieve the maximum percent identity (i.e., gaps can be introduced in one or both of the candidate and reference sequences for optimal alignment and non-homologous sequences can be disregarded for comparison purposes). Alignment for purposes of determining percent identity can be achieved in various ways that are within the skill in the art, for instance, using publicly available computer software such as BLAST, ALIGN, or Megalign (DNASTAR) software. Those skilled in the art can determine appropriate parameters for measuring alignment, including any algorithms needed to achieve maximal alignment over the full length of the sequences being compared. In some embodiments, the percent amino acid sequence identity of a given candidate sequence to, with, or against a given reference sequence (which can alternatively be phrased as a given candidate sequence that has or includes a certain percent amino acid sequence identity to, with, or against a given reference sequence) is calculated as follows:

100×(fraction of A/B)

where A is the number of amino acid residues scored as identical in the alignment of the candidate sequence and the reference sequence, and where B is the total number of amino acid residues in the reference sequence. In some embodiments where the length of the candidate sequence does not equal to the length of the reference sequence, the percent amino acid sequence identity of the candidate sequence to the reference sequence would not equal to the percent amino acid sequence identity of the reference sequence to the candidate sequence.

Two polynucleotide or polypeptide sequences are said to be “identical” if the sequence of nucleotides or amino acids in the two sequences is the same when aligned for maximum correspondence as described above. Comparisons between two sequences are typically performed by comparing the sequences over a comparison window to identify and compare local regions of sequence similarity. A “comparison window” as used herein, refers to a segment of at least about 15 contiguous positions, about 20 contiguous positions, about 25 contiguous positions, or more (e.g., about 30 to about 75 contiguous positions, or about 40 to about 50 contiguous positions), in which a sequence may be compared to a reference sequence of the same number of contiguous positions after the two sequences are optimally aligned.

The term “treating” or “to treat,” as used herein, refers to a therapeutic treatment of a viral infection in a subject. In some embodiments, a therapeutic treatment may slow the progression of the viral infection, improve the subject's outcome, and/or eliminate the infection. In some embodiments, a therapeutic treatment of a viral infection in a subject may alleviate or ameliorate of one or more symptoms or conditions associated with the viral infection, diminish the extent of the viral, stabilize (i.e., not worsening) the state of the viral infection, prevent the spread of the viral infection, and/or delay or slow the progress of the viral infection, as compare the state and/or the condition of the viral infection in the absence of the therapeutic treatment.

The term “average value of T,” as used herein, refers to the mean number of dimers of neuraminidase inhibitors conjugated to an Fc domain within a population of conjugates. In some embodiments, within a population of conjugates, the average number of dimers of neuraminidase inhibitors conjugated to an Fc domain monomer may be from 1 to 20 (e.g., the average value of T is 1 to 2, 1 to 3, 1 to 4, 1 to 5, 5 to 10, 10 to 5, 15 to 20, 1.5 to 3.5, 2.5 to 4.5, 3.5 to 5.5, 4.5 to 6.5, 5.5 to 7.5, 6.5 to 8.5, 7.5 to 9.5, or 8.5 to 10.5). In some embodiments, the average value of T is 1, 2, 3, 4, 5, 6, 7, 8, 9, 10, 11, 12, 13, 14, 15, 16, 17, 18, 19, or 20.

The term “subject,” as used herein, can be a human, non-human primate, or other animal, such as but not limited to dog, cat, horse, cow, pig, turkey, goat, fish, monkey, chicken, rat, mouse, and sheep.

The term “therapeutically effective amount,” as used herein, refers to an amount, e.g., pharmaceutical dose, effective in inducing a desired effect in a subject or in treating a subject having a condition or disorder described herein (e.g., a viral infection, such as an influenza infection). It is also to be understood herein that a “therapeutically effective amount” may be interpreted as an amount giving a desired therapeutic and/or preventative effect, taken in one or more doses or in any dosage or route, and/or taken alone or in combination with other therapeutic agents (e.g., an antiviral agent described herein). For example, in the context of administering a conjugate described herein (e.g., a conjugate of any one of formulas (M-I) or (D-I)-(D-VIII)) that is used for the treatment of a viral infection, an effective amount of a conjugate is, for example, an amount sufficient to prevent, slow down, or reverse the progression of the viral infection as compared to the response obtained without administration of the conjugate.

As used herein, the term “pharmaceutical composition” refers to a medicinal or pharmaceutical formulation that contains at least one active ingredient (e.g., a conjugate of any one of formulas (M-I) or (D-I)-(D-VIII)) as well as one or more excipients and diluents to enable the active ingredient suitable for the method of administration. The pharmaceutical composition of the present disclosure includes pharmaceutically acceptable components that are compatible with a conjugate described herein (e.g., a conjugate of any one of formulas (M-I) or (D-I)-(D-VIII)).

As used herein, the term “pharmaceutically acceptable carrier” refers to an excipient or diluent in a pharmaceutical composition. For example, a pharmaceutically acceptable carrier may be a vehicle capable of suspending or dissolving the active conjugate (e.g., a conjugate of any one of formulas (M-I) or (D-I)-(D-VIII)). The pharmaceutically acceptable carrier must be compatible with the other ingredients of the formulation and not deleterious to the recipient. In the present disclosure, the pharmaceutically acceptable carrier must provide adequate pharmaceutical stability to a conjugate described herein. The nature of the carrier differs with the mode of administration. For example, for oral administration, a solid carrier is preferred; for intravenous administration, an aqueous solution carrier (e.g., WFI, and/or a buffered solution) is generally used.

The term “pharmaceutically acceptable salt,” as used herein, represents salts of the conjugates described herein (e.g., conjugates of any one of formulas (M-I) or (D-I)-(D-VIII)) that are, within the scope of sound medical judgment, suitable for use in methods described herein without undue toxicity, irritation, and/or allergic response. Pharmaceutically acceptable salts are well known in the art. For example, pharmaceutically acceptable salts are described in: Pharmaceutical Salts: Properties, Selection, and Use (Eds. P. H. Stahl and C. G. Wermuth), Wiley-VCH, 2008. The salts can be prepared in situ during the final isolation and purification of the conjugates described herein or separately by reacting the free base group with a suitable organic acid.

The term “drug-to-antibody ratio” or “DAR” refers to the average number of small molecule drug moieties (e.g., the average number of small molecule drug dimers) conjugated to an antibody or Fc domain, described herein. In some embodiments described herein, the DAR is represented by “T” (e.g., in formulas (M-I) or (D-I)-(D-VIII)). As used herein, each dimer moiety (e.g., each zanamivir dimer) conjugated to the Fc domain or antibody corresponds to a DAR or “T” value of 1.0. DAR values may affect the efficacy, potency, pharmacokinetics, or toxicity of the drug.

The term “secondary infection,” as used herein, refers to an infection that occurs in a subject during or after another (referred to as primary) infection in that subject (e.g., during or after a primary influenza infection). A secondary infections may be caused by the primary infection or may be caused by treatment of the primary infection. In some cases, primary infections alter the immune system making the subject more susceptible to a secondary infection. In some cases, treatment of the primary infection makes the subject more susceptible to a secondary infection. For example, the influenza virus has been associated with secondary infections (e.g., increased risk of developing a secondary infection), such as bacterial secondary infections, for example of the respiratory tract. Secondary infections associated with influenza infection increase the morbidity and mortality of influenza. Secondary infections include co-infections. The terms “secondary infection” and “co-infection” are used interchangeably herein.

The term “about,” as used herein, indicates a deviation of up to ±5%. For example, about 10% refers to from 9.5% to 10.5%.

Any values provided in a range of values include both the upper and lower bounds, and any values contained within the upper and lower bounds.

The term “(D-I)-(D-VIII)”, as used herein, represents the formulas of any one of (D-I), (D-II), (D-II-1), (D-II-2), (D-II-3), (D-II-4), (D-II-5), (D-II-6), (D-II-7), (D-II-8), (D-II-9), (D-II-10), (D-Ill), (D-IV), (D-V), (D-VI), (D-VII), and (D-VIII).

Other features and advantages of the conjugates described herein will be apparent from the following Detailed Description and the claims.

### DETAILED DESCRIPTION

The disclosure features methods and intermediates for synthesizing conjugates useful for the treatment of viral infections (e.g., influenza viral infections) and related conditions. The conjugates disclosed herein include dimers of viral neuraminidase inhibitors (e.g., zanamivir or analogs thereof) conjugated to Fc monomers, Fc domains, or albumin protein. The anti-influenza moiety (e.g., neuraminidase inhibitor) (e.g., zanamivir or analogs thereof) in the conjugates targets neuraminidase on the surface of the viral particle. The Fc monomers or Fc domains in the conjugates bind to FcγRs (e.g., FcRn, FcγRI, FcγRIIa, FcγRIIc, FcγRIIIa, and FcγRIIIb) on immune cells, e.g., neutrophils, to activate phagocytosis and effector functions, such as antibody-dependent cell-mediated cytotoxicity (ADCC), thus leading to the engulfment and destruction of viral particles by immune cells and further enhancing the antiviral activity of the conjugates. The albumin or albumin-binding peptide may extend the half-life of the conjugate, for example, by binding of albumin to the recycling neonatal Fc receptor. Such compositions are useful in methods for the inhibition of viral growth and in methods for the treatment of viral infections, such as those caused by an influenza virus A, influenza virus B and influenza virus C.

The compounds and methods described herein are valuable in generating conjugates useful for the treatment of viral infections (e.g. influenza infections) and conditions thereof.

The methods disclosed herein can provide a number of advantages, such as higher overall yield and higher purity (e.g., efficient elimination of impurities) of the final product (e.g., a conjugate of formula (D-I) or (M-I)), as well as reduced waste stream (e.g., reducing the total number of reaction steps or reducing loss of starting material (e.g., E and/or compound of formula (DF-I), (DF-II), (MF-I), or (MF-II)) and mild reaction conditions (e.g., step (c) or step (e) of the methods described herein). The methods of the disclosure can also enable reliable synthesis of the final product (e.g., a conjugate of formula (D-I) or (M-I)) having preferred characteristics, e.g., drug-to-antibody ratio (DAR).

### I. Viral Infections

The conjugates described herein (e.g., a conjugate of formula (D-I) or (M-I)) can be used to treat a viral infection (e.g., an influenza viral infection, such as influenza A, B, C, or parainfluenza).

Viral infection refers to the pathogenic growth of a virus (e.g., the influenza virus) in a host organism (e.g., a human subject). A viral infection can be any situation in which the presence of a viral population(s) is damaging to a host body. Thus, a subject is suffering from a viral infection when an excessive amount of a viral population is present in or on the subject's body, or when the presence of a viral population(s) is damaging the cells or other tissue of the subject.

Influenza, commonly known as “the flu”, is an infectious disease caused by an influenza virus. Symptoms can be mild to severe. The most common symptoms include: a high fever, runny nose, sore throat, muscle pains, headache, coughing, and feeling tired. These symptoms typically begin two days after exposure to the virus and most last less than a week. The cough, however, may last for more than two weeks. In children, there may be nausea and vomiting, but these are less common in adults. Complications of influenza may include viral pneumonia, secondary bacterial pneumonia, sinus infections, and worsening of previous health problems such as asthma or heart failure. Severe complications may occur in subjects having weakened immune systems, such as the young, the old, those with illnesses that weaken the immune system, and those undergoing therapy treatment resulting in a weakening of the immune system.

Subjects infected with influenza are also at increased risk of developing secondary infections (e.g., secondary bacterial, viral, or fungal infections), in particular, bacterial infections such as methicillin-resistant Staphylococcus aureus (MRSA), Streptococcus pneumoniae, Pseudomonas aeruginosa, and/or Haemophilus influenzae. Bacterial secondary infections further increase morbidity and mortality of influenza infection.

Three types of influenza viruses affect human subjects, namely Type A, Type B, and Type C. Usually, the virus is spread through the air from coughs or sneezes. This is believed to occur mostly over relatively short distances. It can also be spread by touching surfaces contaminated by the virus and then touching the mouth or eyes. A person may be infectious to others both before and during the time they are showing symptoms. The infection may be confirmed by testing the throat, sputum, or nose for the virus. A number of rapid tests are available; however, people may still have the infection if the results are negative. A type of polymerase chain reaction that detects the virus's RNA may be used to diagnose influenza infection.

### II. Conjugates of the Disclosure

Described herein are synthetic conjugates useful in the treatment of viral infections (e.g., influenza infections). The conjugates described herein include an Fc domain or an albumin protein conjugated to one or more anti-influenza moieties.

In some embodiments, the conjugates described herein include an Fc domain or albumin protein conjugated to one or more dimers of two anti-influenza moieties (e.g., neuraminidase inhibitors selected from zanamivir or analogs thereof). For example, the dimers of two anti-influenza moieties includes a first anti-influenza moiety (e.g., of formula (A-I)-(A-XIII)) and a second anti-influenza moiety (e.g., of formula (A-I)-(A-XIII)). The first and second neuraminidase inhibitors are linked to each other by way of a linker.

In some embodiments, the conjugates described herein include an Fc domain or albumin protein conjugated to one or more monomers of an anti-influenza moiety (e.g., of formula (A-I)-(A-XIII)).

Without being bound by theory, in some aspects, conjugates described herein bind to the surface of a viral particle (e.g., bind to viral neuraminidase enzyme on the surface on an influenza viral particle) through the interactions between the neuraminidase inhibitor moieties in the conjugates and proteins on the surface of the viral particle. The neuraminidase inhibitor disrupts neuraminidase, an envelope glycoprotein that cleaves sialic acids, i.e., terminal neuraminic acid residues, from glycan structures on the surface of infected host cells, releasing progeny viruses and allowing the spread of the virus from the host cell to uninfected surrounding cells.

Conjugates of the disclosure include neuraminidase inhibitor dimers conjugated to an Fc domain or Fc monomer. The Fc domain in the conjugates described herein binds to the FcγRs (e.g., FcRn, FcγRI, FcγRIIa, FcγRIIc, FcγRIIIa, and FcγRIIIb) on immune cells. The binding of the Fc domain in the conjugates described herein to the FcγRs on immune cells activates phagocytosis and effector functions, such as antibody-dependent cell-mediated cytotoxicity (ADCC), thus leading to the engulfment and destruction of viral particles by immune cells and further enhancing the antiviral activity of the conjugates.

In some embodiments, the conjugates described herein include one or more dimers of neuraminidase inhibitors conjugated to an Fc domain. In some embodiments, when n is 2, E (an Fc domain monomer) dimerizes to form an Fc domain.

Conjugates described herein may be synthesized using available chemical synthesis techniques in the art. In cases where a functional group is not available for conjugation, a molecule may be derivatized using conventional chemical synthesis techniques that are well known in the art. In some embodiments, the conjugates described herein contain one or more chiral centers. The conjugates include each of the isolated stereoisomeric forms as well as mixtures of stereoisomers in varying degrees of chiral purity, including racemic mixtures. It also encompasses the various diastereomers, enantiomers, and tautomers that can be formed.

### Anti-Influenza Moieties

In some embodiments, a component of the conjugates described herein is an anti-influenza moiety. An anti-influenza moiety is a compound or composition that inhibits a function of the influenza virus. For example, and anti-influenza moiety may inhibit viral entry into a host cell or inhibit viral replication or proliferation. An anti-influenza moiety may target the virus or the host cell (e.g., a viral protein or a receptor on the host cell). In some embodiments, the anti-influenza moiety is a small molecule. Small molecule inhibitors of influenza (e.g., inhibitors of host cell entry or viral replication of influenza) are known in the art and include, for example, pimovidir, oseltamivir, zanamivir, sulfozanamivir, peramivir, Ianinamivir, amantadine, rimantadine, baloxavir (e.g., baloxavir acid or baloxavir marboxil), and chemical analogs thereof. Methods for measuring anti-viral activity are known in the art. An anti-influenza moiety may inhibit influenza A, B, C, parainfluenza, or any combination thereof.

### Neuraminidase Inhibitors

In some embodiments, a component of the conjugates described herein is an influenza virus neuraminidase inhibitor moiety. An influenza virus neuraminidase inhibitor disrupts neuraminidase, an envelope glycoprotein that cleaves sialic acids, i.e., terminal neuraminic acid residues, from glycan structures on the surface of infected host cells, releasing progeny viruses and allowing the spread of the virus from the host cell to uninfected surrounding cells. Examples of an influenza virus neuraminidase inhibitor include zanamivir (Relenza), sulfozanamivir, peramivir, and analogs thereof that retain neuraminidase inhibitor binding and/or activity.

Viral neuraminidase inhibitors of the disclosure include zanamivir, sulfozanamivir, peramivir, and analogs thereof, such as the viral neuraminidase inhibitors of formulas (A-I)-(A-XIII).

Preferably the viral neuraminidase inhibitor is selected from zanamivir or sulfozanamivir:

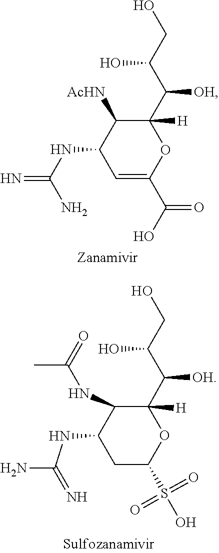 [SMILES: CC(=O)N[C@H]1[C@H]([C@H](O)[C@H](O)CO)OC(C(=O)O)=C[C@@H]1NC(=N)N ; CC(=O)N[C@H]1[C@H]([C@H](O)[C@H](O)CO)O[C@H](S(=O)(=O)O)C[C@@H]1NC(=N)N]

### Conjugates of Monomers of Anti-Influenza Moieties (e.g., Neuraminidase Inhibitors) Linked to an Fc Domain or an Albumin Protein

In some embodiments, the conjugates described herein include an Fc domain monomer, Fc domain, Fc-binding peptide, albumin protein, or albumin protein-binding peptide covalently linked to one or more monomers of an anti-influenza moiety. Conjugates of an Fc domain monomer or albumin protein and one or more monomers of an anti-influenza moiety may be formed by linking the Fc domain or albumin protein to each of the monomers of RSV F protein inhibitors through a linker, such as any of the linkers described herein.

In the conjugates having an Fc domain or albumin protein covalently linked to one or more monomers of an anti-influenza moiety described herein, the squiggly line connected to E indicates that one or more (e.g., 1, 2, 3, 4, 5, 6, 7, 8, 9, 10, 11, 12, 13, 14, 15, 16, 17, 18, 19, or 20) monomers of an anti-influenza moiety may be attached to an Fc domain monomer, Fc domain, Fc-binding peptide, albumin protein, or albumin protein-binding peptide. In some embodiments, when n is 1, one or more (e.g., 1, 2, 3, 4, 5, 6, 7, 8, 9, or 10) monomers of an anti-influenza moiety may be attached to an Fc domain monomer or an albumin protein. In some embodiments, when n is 2, one or more (e.g., 1, 2, 3, 4, 5, 6, 7, 8, 9, 10, 11, 12, 13, 14, 15, 16, 17, 18, 19, or 20) monomers of an anti-influenza moiety may be attached to an Fc domain. The squiggly line in the conjugates described herein is not to be construed as a single bond between one or more monomers of an anti-influenza moiety and an atom in the Fc domain monomer, Fc domain, Fc-binding peptide, albumin protein, or albumin protein-binding peptide. In some embodiments, when T is 1, one monomer of an anti-influenza moiety may be attached to an atom in the Fc domain monomer, Fc domain, Fc-binding peptide, albumin protein, or albumin protein-binding peptide. In some embodiments, when T is 2, two monomers of an anti-influenza moiety may be attached to an atom in the Fc domain monomer, Fc domain, Fc-binding peptide, albumin protein, or albumin protein-binding peptide.

In some embodiments, when T is greater than 1 (e.g., T is 2, 3, 4, 5, 6, 7, 8, 9, 10, 11, 12, 13, 14, 15, 16, 17, 18, 19, or 20), each A₁-L may be independently selected (e.g., independently selected from any of the A₁-L structures described herein). In some embodiments, E may be conjugated to 2, 3, 4, 5, 6, 7, 8, 9, 10, or more different A₁-L moieties. In some embodiments, E is conjugated to a first A₁-L moiety, and a second A₁-L, moiety. In some embodiments, A₁ of each of the first A₁-L moiety and the second A₁-L moiety is independently selected from any one of formulas (A-I)-(A-XIII).

In some embodiments, the first A₁-L moiety is conjugated specifically to lysine residues of E (e.g., the nitrogen atoms of surface exposed lysine residues of E), and the second A₁-L moiety is conjugated specifically to cysteine residues of E (e.g., the sulfur atoms of surface exposed cysteine residues of E). In some embodiments, the first A₁-L moiety is conjugated specifically to cysteine residues of E (e.g., the sulfur atoms of surface exposed cysteine residues of E), and the second A₁-L moiety is conjugated specifically to lysine residues of E (e.g., the nitrogen atoms of surface exposed lysine residues of E).

As described further herein, a linker in a conjugate having an Fc domain monomer, Fc domain, Fc-binding peptide, albumin protein, or albumin protein-binding peptide covalently linked to one or more monomers of an anti-influenza moiety described herein (e.g., L or L′) may be a divalent structure having two arms. One arm in a divalent linker may be attached to the monomer of an anti-influenza moiety and the other arm may be attached to the Fc domain monomer, Fc domain, Fc-binding peptide, albumin protein, or albumin protein-binding peptide.

In conjugates having an Fc domain covalently linked to one or more monomers of an anti-influenza moiety, as represented by the formulae above, when n is 2, two Fc domain monomers (each Fc domain monomer is represented by E) dimerize to form an Fc domain.

### Conjugates of Dimers of Anti-Influenza Moieties (e.g., Neuraminidase Inhibitors) Linked to an Fc Domain or an Albumin Protein

The conjugates described herein include an Fc domain or an Fc monomer covalently linked to one or more dimers of an anti-influenza moiety. The dimers of two anti-influenza moieties include a first anti-influenza moiety (e.g., a first viral neuraminidase inhibitor of formulas (A-I)-(A-VIII)) and a second anti-influenza moiety (e.g., a second viral neuraminidase inhibitor of formulas (A-I)-(A-XIII)). The first and second anti-influenza moieties are linked to each other by way of a linker, such as a linker described herein. In some embodiments of the dimers of anti-influenza moieties, the first and second anti-influenza moieties are the same. In some embodiments, the first and second anti-influenza moieties are different.

In the conjugates described herein, the squiggly line connected to E indicates that one or more (e.g., 1, 2, 3, 4, 5, 6, 7, 8, 9, 10, 11, 12, 13, 14, 15, 16, 17, 18, 19, or 20) dimers of an anti-influenza moiety may be attached to an Fc domain monomer or Fc domain. In some embodiments, when n is 1, one or more (e.g., 1, 2, 3, 4, 5, 6, 7, 8, 9, or 10) dimers of an anti-influenza moiety may be attached to an Fc domain monomer or Fc domain. In some embodiments, when n is 2, one or more (e.g., 1, 2, 3, 4, 5, 6, 7, 8, 9, 10, 11, 12, 13, 14, 15, 16, 17, 18, 19, or 20) dimers of an anti-influenza moiety may be attached to an Fc domain. The squiggly line in the conjugates described herein is not to be construed as a single bond between one or more dimers of an anti-influenza moiety and an atom in the Fc domain. In some embodiments, when T is 1, one dimer of an anti-influenza moiety may be attached to an atom in the Fc domain monomer or Fc domain. In some embodiments, when T is 2, two dimers of an anti-influenza moiety may be attached to an atom in the Fc domain monomer or Fc domain.

As described further herein, a linker in a conjugate described herein (e.g., L or L′) may be a branched structure. As described further herein, a linker in a conjugate described herein (e.g., L or L′) may be a multivalent structure, e.g., a divalent or trivalent structure having two or three arms, respectively. In some embodiments when the linker has three arms, two of the arms may be attached to the first and second anti-influenza moiety and the third arm may be attached to the Fc domain monomer or Fc domain.

In conjugates having an Fc domain covalently linked to one or more dimers of an anti-influenza moiety, as represented by the formulae above, when n is 2, two Fc domain monomers (each Fc domain monomer is represented by E) dimerize to form an Fc domain.

### Regioisomers of Conjugates Including Zanamivir or Analogs Thereof

Conjugates may be produced as a mixture or regioisomers. A particular regioisomer or mixture of regioisomers may be preferred for reasons such as ease of synthesis, thermostability, oxidative stability, pharmacokinetics (e.g., metabolic stability or bioavailability), effector binding, or therapeutic efficacy.

In some embodiments, a conjugate of the disclosure includes zanamivir or an analog thereof (e.g., any of (A-I)-(A-VIII)). Zanamivir or an analog thereof may be conjugated to an Fc domain (e.g., by way of a linker) through, for example, the C7 position (see, e.g., (A-I), (A-II), (A-VII), or (A-VIII)) or through the C9 position (see, e.g., (A-III) or (A-IV)):

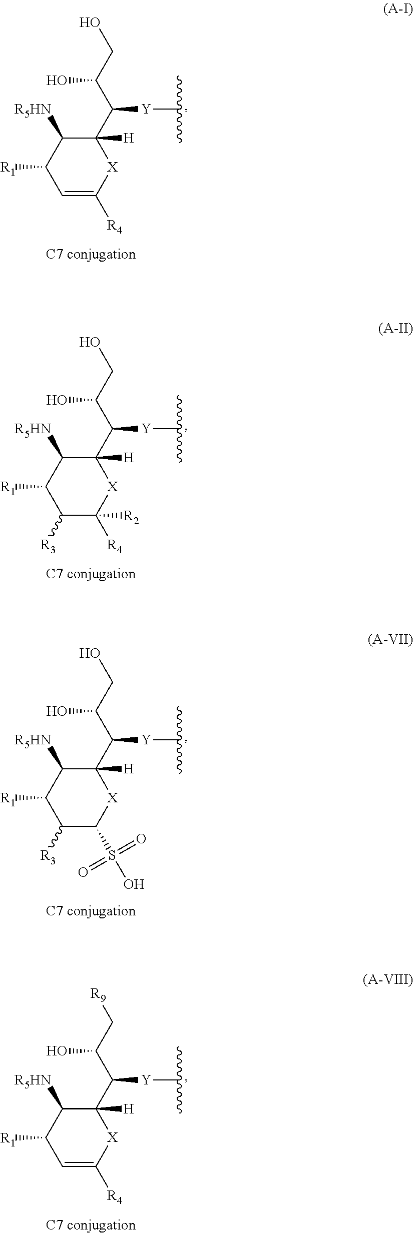

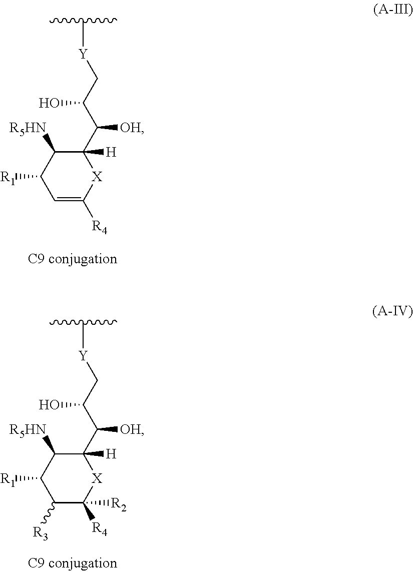

The present disclosure describes a population of conjugates (e.g., a population of conjugates of any one of formulas (M-I) or (D-I)-(D-VIII)) wherein the population of conjugates includes any of the monomeric or dimeric conjugates described herein and one or more of its corresponding regioisomers. For example, a population of conjugates may include a (1) a C7 monomer or C7-C7 dimer (e.g., both zanamivir or analog thereof moieties of the dimer are conjugated (e.g., by way of a linker) at their respective C7 positions to an Fc domain), (2) a C9 monomer or a C9-C9 dimer (e.g., both zanamivir or analog thereof moieties of the dimer are conjugated (e.g., by way of a linker) at their respective C9 positions to an Fc domain), and/or (3) a C7-C9 dimer (e.g., one zanamivir or analog thereof moiety is conjugated (e.g., by way of a linker) to and Fc domain through its C7 position and the other zanamivir or analog thereof moiety is conjugated (e.g., by way of a linker) to an Fc domain through its C9 position).

The population of dimeric conjugates may have a specified ratio of C7-C7 linked conjugate to C7-C9 linked conjugate to C9-C9 linked conjugate. For example, the population of conjugates may have substantially 100% C7-C7 linked conjugate, and substantially 0% C7-C9 or C9-C9 linked conjugate. The population of conjugates may have substantially 100% C9-C9 linked conjugate, and substantially 0% C7-C7 or C7-C9 linked conjugate. The population of conjugates may have substantially 100% C7-C9 linked conjugate, and substantially 0% C7-C7 or C9-C9 linked conjugate.

The population of conjugates may have greater than 99%, 98%, 97%, 96%, 95%, 90%, 85%, 80%, 75%, 70%, 60%, 65%, 60%, 55%, or 50% C7-C7 linked conjugate.

The population of conjugates may have less than 50%, 40%, 30%, 25%, 20%, 15%, 10%, 5%, 4%, 3%, 2%, or 1% C9-C9 linked conjugate.

The population of conjugates may have less than 50%, 40%, 30%, 25%, 20%, 15%, 10%, 5%, 4%, 3%, 2%, or 1% C7-C9 linked conjugate.

For any of the above-described populations of regioisomers, A₁ and/or A₂ may be selected from zanamivir or any of the zanamivir analogs described herein (e.g., any of (A-I)-(A-VIII)). In particular, the C7-linked zanamivir or analogs thereof is described by (A-I), (A-II), (A-VII), and (A-VIII), and C9-linked zanamivir or analogs thereof is described by (A-III) or (A-IV). In some instances, it may be preferable to have 95% or more, 96% or more, 97% or more, 98% or more, 99% or more, or substantially 100% C7-C7 linked dimer conjugates. In these instances, it may be preferable to prepare the intermediate with a method that forms substantially C7-C7 linked dimer intermediates.

Zanamivir analogs having a modification (e.g., a substituent other than OH) at position C9 (e.g., zanamivir analogs described by (A-XIII)) may increase the ratio of C7-linked zanamivir to C9-linked zanamivir by preventing the migration from C7-linked zanamivir to C9-linked zanamivir. Exemplary C9-modified zanamivir analogs are described herein (see, e.g., conjugates described by D-XI or M-XI).

In preferred embodiments, the conjugate is a conjugate of any one of formulas (M-I) or (D-I)-(D-VIII), wherein A₁ and/or A₂ are described by formula (A-I), (A-II), (A-VII), or (A-VIII) and Y is

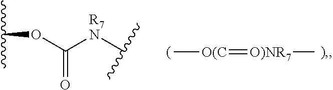

wherein R₇ is selected from H, C1-C20 alkyl, C3-C20 cycloalkyl, C3-C20 heterocycloalkyl; C5-C15 aryl, and C2-C15 heteroaryl. In preferred embodiments, A₁ and/or A₂ are described by formula (A-I) (e.g., zanamivir). In preferred embodiments, R₇ is C1-C20 alkyl (e.g., —CH₃, —CH₂CH₃, —CH₂CH₂CH₃). Such conjugates have been shown to exhibit increased stability of the C7-linkage, resulting in less C7 to C9 migration (see, e.g., conjugates described by D-II-6 or D-II-7). The resulting product is therefore expected to be more homogenous and exhibit increased efficacy. The preferred conjugate is more homogenous, has an increased proportion (e.g., substantially pure, such as greater than 95%, 96%, 97%, 98%, or 99% pure) C7-linked zanamivir, and retains efficacy against influenza.

Exemplary syntheses of intermediates including zanamivir or analogs thereof are described in WO 2020/051498 and WO 2021/046549, each of which is hereby incorporated by reference.

### III. Fc Domain Monomers and Fc Domains

An Fc domain monomer includes a hinge domain, a C_{H}2 antibody constant domain, and a C_{H}3 antibody constant domain. The Fc domain monomer can be of immunoglobulin antibody isotype IgG, IgE, IgM, IgA, or IgD. The Fc domain monomer can also be of any immunoglobulin antibody isotype (e.g., IgG1, IgG2a, IgG2b, IgG3, or IgG4). The Fc domain monomer can be of any immunoglobulin antibody allotype (e.g., IGHG1*01 (i.e., G1m(za)), IGHG1*07 (i.e., G1m(zax)), IGHG1*04 (i.e., G1m(zav)), IGHG1*03 (G1m(f)), IGHG1*08 (i.e., G1m(fa)), IGHG2*01, IGHG2*06, IGHG2*02, IGHG3*01, IGHG3*05, IGHG3*10, IGHG3*04, IGHG3*09, IGHG3*11, IGHG3*12, IGHG3*06, IGHG3*07, IGHG3*08, IGHG3*13, IGHG3*03, IGHG3*14, IGHG3*15, IGHG3*16, IGHG3*17, IGHG3*18, IGHG3*19, IGHG2*04, IGHG4*01, IGHG4*03, or IGHG4*02) (as described in, for example, in Vidarsson et al. IgG subclasses and allotypes: from structure to effector function. Frontiers in Immunology. 5(520):1-17 (2014)). The Fc domain monomer can also be of any species, e.g., human, murine, or mouse. A dimer of Fc domain monomers is an Fc domain that can bind to an Fc receptor, which is a receptor located on the surface of leukocytes. In some embodiments, an Fc domain monomer in the conjugates described herein may contain one or more amino acid substitutions, additions, and/or deletion relative to an Fc domain monomer having a sequence of any one of SEQ ID NOs: 1-14. In some embodiments, an Asn in an Fc domain monomer in the conjugates as described herein may be replaced by Ala in order to prevent N-linked glycosylation. In some embodiments, an Fc domain monomer in the conjugates described herein may also containing additional Cys additions.

In some embodiments, an Fc domain monomer in the conjugates as described herein includes an additional moiety, e.g., an albumin-binding peptide, a purification peptide (e.g., a hexa-histidine peptide), or a signal sequence (e.g., IL2 signal sequence) attached to the N- or C-terminus of the Fc domain monomer. In some embodiments, an Fc domain monomer in the conjugate does not contain any type of antibody variable region, e.g., V_{H}, V_{L}, a complementarity determining region (CDR), or a hypervariable region (HVR).

In some embodiments, an Fc domain monomer in the conjugates as described herein may have a sequence that is at least 95% identical (e.g., 97%, 99%, or 99.5% identical) to the sequence of any one of SEQ ID NOs: 1-14 shown below. In some embodiments, an Fc domain monomer in the conjugates as described herein may have a sequence of any one of SEQ ID NOs: 1-14 shown below.

SEQ ID NO: 1: mature human Fc IgG1, Z₁ is Cys or Ser, and wherein X₁ is Met or Tyr, X₂ is Ser or
Thr, X₃ is Thr or Glu, X₄ is Asp or Glu, and X₅ is Leu or Met, X₆ is Met or Leu, and X₇ is Asn or
Ser
NVNHKPSNTKVDKKVEPKSZ₁DKTHTCPPCPAPELLGGPSVFLFPPKPKDTLX₁IX₂RX₃PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVX₆HEALHX₇HYTQKSLSLSPGK
SEQ ID NO: 2: mature human Fc IgG1, Cys to Ser substitution (#), and wherein X₁ is Met or Tyr,
X₂ is Ser or Thr, X₃ is Thr or Glu, X₄ is Asp or Glu, and X₅ is Leu or Met, X₆ is Met or Leu, and
X₇ is Asn or Ser
NVNHKPSNTKVDKKVEPKSS(#)DKTHTCPPCPAPELLGGPSVFLFPPKPKDTLX₁IX₂RX₃PEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVX₆HEALHX₇HYTQKSLSLSPGK
SEQ ID NO: 3: mature human IgG1 Fc, Cys to Ser substitution (#), X₄ is Asp or Glu, and X₅ is
Leu or Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVMHEALHNHYTQKSLSLSPGK
SEQ ID NO: 4: mature human IgG1 Fc, Cys to Ser substitution (#), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 5: mature human IgG1 Fc, Cys to Ser substitution (#), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 6: mature human IgG1 Fc, Cys to Ser substitution (#), YTE triple mutation (bold and
underlined), allotype G1m(fa) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 7: mature human IgG1 Fc, Cys to Ser substitution (#), YTE triple mutation (bold and
underlined), allotype G1m(f) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPGK
SEQ ID NO: 8: mature human Fc IgG1, Z₁ is Cys or Ser, and wherein X₁ is Met or Tyr, X₂ is Ser or
Thr, X₃ is Thr or Glu, X₄ is Asp or Glu, and X₅ is Leu or Met, X₆ is Met or Leu, and X₇ is Asn
or Ser
NVNHKPSNTKVDKKVEPKSZ₁DKTHTCPPCPAPELLGGPSVFLFPPKPKDTLX₁IX₂RX₃PEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVX₆HEALHX₇HYTQKSLSLSPG
SEQ ID NO: 9: mature human Fc IgG1, Cys to Ser substitution (#), and wherein X₁ is Met or Tyr,
X₂ is Ser or Thr, X₃ is Thr or Glu, X₄ is Asp or Glu, and X₅ is Leu or Met, X₆ is Met or Leu, and
X₇ is Asn or Ser
NVNHKPSNTKVDKKVEPKSS(#)DKTHTCPPCPAPELLGGPSVFLFPPKPKDTLX₁IX₂RX₃PEVTCVVVDVS
HEDPEVKFNWYVDGVEVHNAKTKPREEQYNSTYRVVSVLTVLHQDWLNGKEYKCKVSNKALPAPIEKTI
SKAKGQPREPQVYTLPPSRX₄EX₅TKNQVSLTCLVKGFYPSDIAVEWESNGQPENNYKTTPPVLDSDGSF
FLYSKLTVDKSRWQQGNVFSCSVX₆HEALHX₇HYTQKSLSLSPG
SEQ ID NO: 10: mature human IgG1 Fc, Cys to Ser substitution (#), X₄ is Asp or Glu, and X₅ is
Leu or Met
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSRX₄EX₅TKNQVSLTCLVKGFYPSDIAVEWESNGQPENNYKTTPPVLDSDGSFF
LYSKLTVDKSRWQQGNVFSCSVMHEALHNHYTQKSLSLSPG
SEQ ID NO: 11: mature human IgG1 Fc, Cys to Ser substitution (#), allotype G1m(f) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 12: mature human IgG1 Fc, Cys to Ser substitution (#), allotype G1m(fa) (bold italics)
NVNHKPSNTKVDKKVEPKSS(#)DKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 13: mature human IgG1 Fc, Cys to Ser substitution (#), YTE triple mutation (bold and
underlined), allotype G1m(fa) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG
SEQ ID NO: 14: mature human IgG1 Fc, Cys to Ser substitution (#), YTE triple mutation (bold and
underlined), allotype G1m(f) (bold italics)
NVNHKPSNTKVDKKVEPKSS(#)DKTHTCPPCPAPELLGGPSVFLFPPKPKDTLYITREPEVTCVVVDVSH
EDPEVKFNWYVDGVEVHNAKTKPREEQYNSTYRVVSVLTVLHQDWLNGKEYKCKVSNKALPAPIEKTIS
KAKGQPREPQVYTLPPSR [symbol] E [symbol] TKNQVSLTCLVKGFYPSDIAVEWESNGQPENNYKTTPPVLDSDGSFFL
YSKLTVDKSRWQQGNVFSCSVMHEALHNHYTQKSLSLSPG

As defined herein, an Fc domain includes two Fc domain monomers that are dimerized by the interaction between the C_{H}3 antibody constant domains, as well as one or more disulfide bonds that form between the hinge domains of the two dimerizing Fc domain monomers. An Fc domain forms the minimum structure that binds to an Fc receptor, e.g., Fc-gamma receptors (i.e., Fcγ receptors (FcγR)), Fc-alpha receptors (i.e., Fcα receptors (FcαR)), Fc-epsilon receptors (i.e., Fcε receptors (FcεR)), and/or the neonatal Fc receptor (FcRn). In some embodiments, an Fc domain of the present disclosure binds to an Fcγ receptor (e.g., FcRn, FcγRI (CD64), FcγRIIa (CD32), FcγRIIb (CD32), FcγRIIIa (CD16a), FcγRIIIb (CD16b)), and/or FcγRIV and/or the neonatal Fc receptor (FcRn).

In some embodiments, the Fc domain monomer or Fc domain of the disclosure is an aglycosylated Fc domain monomer or Fc domain (e.g., an Fc domain monomer or and Fc domain that maintains engagement to an Fc receptor (e.g., FcRn). For example, the Fc domain is an aglycosylated IgG1 variants that maintains engagement to an Fc receptor (e.g., an IgG1 having an amino acid substitution at N297 and/or T299 of the glycosylation motif). Exemplary aglycosylated Fc domains and methods for making aglycosylated Fc domains are known in the art, for example, as described in Sazinsky S. L. et al., Aglycosylated immunoglobulin G1 variants productively engage activating Fc receptors, PNAS, 2008, 105(51):20167-20172, which is incorporated herein in its entirety.

In some embodiments, the Fc domain or Fc domain monomer of the disclosure is engineered to enhance binding to the neonatal Fc receptor (FcRn). For example, the Fc domain may include the triple mutation corresponding to M252Y/S254T/T256E (YTE) (e.g., an IgG1, such as a human or humanized IgG1 having a YTE mutation). The Fc domain may include the double mutant corresponding to M428L/N434S (LS) (e.g., an IgG1, such as a human or humanized IgG1 having an LS mutation). The Fc domain may include the single mutant corresponding to N434H (e.g., an IgG1, such as a human or humanized IgG1 having an N434H mutation). The Fc domain may include the single mutant corresponding to C220S (e.g., and IgG1, such as a human or humanized IgG1 having a C220S mutation). The Fc domain may include a quadruple mutant corresponding to C220S/L309D/Q311H/N434S (CDHS) (e.g., an IgG1, such as a human or humanized IgG1 having a CDHS mutation). The Fc domain may include a triple mutant corresponding to L309D/Q211H/N434S (DHS) (e.g., an IgG1, such as a human or humanized IgG1 having a DHS mutation).

The Fc domain may include a combination of one or more of the above-described mutations that enhance binding to the FcRn. Enhanced binding to the FcRn may increase the half-life Fc domain-containing conjugate. For example, incorporation of one or more amino acid mutations that increase binding to the FcRn (e.g., a YTE mutation, an LS mutation, or an N434H mutation) may increase the half life of the conjugate by 5%, 10%, 15%, 20%, 30%, 40%, 50%, 60%, 70%, 80%, 90%, 100%, 200%, 300%, 400%, 500% or more relative to a conjugate having an the corresponding Fc domain without the mutation that enhances FcRn binding. Exemplary Fc domains with enhanced binding to the FcRN and methods for making Fc domains having enhanced binding to the FcRN are known in the art, for example, as described in Maeda, A. et al., Identification of human IgG1 variant with enhanced FcRn binding and without increased binding to rheumatoid factor autoantibody, MABS, 2017, 9(5):844-853, which is incorporated herein in its entirety. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-14 may be mutated to include a YTE mutation, an LS mutation, and/or an N434H mutation by mutating the “corresponding residues” of the amino acid sequence. As used herein, an amino acid “corresponding to” a particular amino acid residue (e.g., of a particular SEQ ID NO.) should be understood to include any amino acid residue that one of skill in the art would understand to align to the particular residue (e.g., of the particular sequence). For example, any one of SEQ ID NOs: 1-14 may be mutated to include a YTE mutation, an LS mutation, and/or an N434H mutation by mutating the “corresponding residues” of the amino acid sequence.

As used herein, a sulfur atom “corresponding to” a particular cysteine residue of a particular SEQ ID NO. should be understood to include the sulfur atom of any cysteine residue that one of skill in the art would understand to align to the particular cysteine of the particular sequence.

As used herein, a nitrogen atom “corresponding to” a particular lysine residue of a particular SEQ ID NO. should be understood to include the nitrogen atom of any lysine residue that one of skill in the art would understand to align to the particular lysine of the particular sequence.

### Activation of Immune Cells

Fc-gamma receptors (FcγRs) bind the Fc portion of immunoglobulin G (IgG) and play important roles in immune activation and regulation. For example, the IgG Fc domains in immune complexes (ICs) engage FcγRs with high avidity, thus triggering signaling cascades that regulate immune cell activation. The human FcγR family contains several activating receptors (FcγRI, FcγRIIa, FcγRIIc, FcγRIIIa, and FcγRIIIb) and one inhibitory receptor (FcγRIIb). FcγR signaling is mediated by intracellular domains that contain immune tyrosine activating motifs (ITAMs) for activating FcγRs and immune tyrosine inhibitory motifs (ITIM) for inhibitory receptor FcγRIIb. In some embodiments, FcγR binding by Fc domains results in ITAM phosphorylation by Src family kinases; this activates Syk family kinases and induces downstream signaling networks, which include PI3K and Ras pathways.

In the conjugates described herein, the portion of the conjugates including dimers of neuraminidase inhibitors bind to and inhibits viral neuraminidase leading to inhibition of viral replication, while the Fc domain portion of the conjugates bind to FcγRs (e.g., FcRn, FcγRI, FcγRIIa, FcγRIIc, FcγRIIIa, and FcγRIIIb) on immune cells and activate phagocytosis and effector functions, such as antibody-dependent cell-mediated cytotoxicity (ADCC), thus leading to the engulfment and destruction of viral particles by immune cells and further enhancing the antiviral activity of the conjugates. Examples of immune cells that may be activated by the conjugates described herein include, but are not limited to, macrophages, neutrophils, eosinophils, basophils, lymphocytes, follicular dendritic cells, natural killer cells, and mast cells.

### Tissue Distribution

After a therapeutic enters the systemic circulation, it is distributed to the body's tissues. Distribution is generally uneven because of different in blood perfusion, tissue binding, regional pH, and permeability of cell membranes. The entry rate of a drug into a tissue depends on the rate of blood flow to the tissue, tissue mass, and partition characteristics between blood and tissue. Distribution equilibrium (when the entry and exit rates are the same) between blood and tissue is reached more rapidly in richly vascularized areas, unless diffusion across cell membranes is the rate-limiting step. The size, shape, charge, target binding, FcRn and target binding mechanisms, route of administration, and formulation affect tissue distribution.

In some instances, the conjugates described herein may be optimized to distribute to lung tissue. In some instances, the conjugates have a concentration ratio of distribution in epithelial lining fluid of at least 30% the concentration of the conjugates in plasma within 2 hours after administration. In certain embodiments, ratio of the concentration is at least 45% within 2 hours after administration. In some embodiments, the ratio of concentration is at least 55% within 2 hours after administration. In particular, the ratio of concentration is at least 60% within 2 hours after administration.

### IV. Albumin Proteins

The disclosure also provides conjugates of an albumin protein with one or more dimers of an anti-influenza moiety (e.g., zanamivir). In particular, the disclosure provides method of making a conjugate including a polypeptide, E, conjugated to one or more anti-influenza moiety (e.g., zanamivir) monomers or dimers, e.g., defined by A₁-L or A₁-L-A₂ as described herein. In some embodiments, E is an albumin protein. In some embodiments, n is 1 and E is an albumin protein.

### Albumin Proteins

An albumin protein may be a naturally-occurring albumin or a variant thereof, such as an engineered variant of a naturally-occurring albumin protein. Variants include polymorphisms, fragments such as domains and sub-domains, and fusion proteins. An albumin protein may include the sequence of an albumin protein obtained from any source. Preferably the source is mammalian, such as human or bovine. Most preferably, the albumin protein is human serum albumin (HSA), or a variant thereof. Human serum albumins include any albumin protein having an amino acid sequence naturally occurring in humans, and variants thereof. An albumin protein coding sequence is obtainable by methods know to those of skill in the art for isolating and sequencing cDNA corresponding to human genes. An albumin protein of the invention may include the amino acid sequence of human serum albumin (HSA), provided in WO 2020/051498, WO 2020/252393, or WO 2020/252396, each of which is hereby incorporated by reference, or the amino acid sequence of mouse serum albumin (MSA), provided in WO 2020/051498, WO 2020/252393, or WO 2020/252396, each of which is hereby incorporated by reference, or a variant or fragment thereof, preferably a functional variant or fragment thereof. A fragment or variant may or may not be functional, or may retain the function of albumin to some degree. For example, a fragment or variant may retain the ability to bind to an albumin receptor, such as HSA or MSA, by at least 10%, 20%, 30%, 40%, 50%, 60%, 70%, 80%, 90%, 100%, or 105% of the ability of the parent albumin (e.g., the parent albumin from which the fragment or variant is derived). Relative binding ability may be determined by methods known in the art, such as by surface plasmon resonance.

The albumin protein may be a naturally-occurring polymorphic variant of an albumin protein, such as human serum albumin. Generally, variants or fragments of human serum albumin will have at least 5%, 10%, 15%, 20%, 30%, 40%, 50%, 60%, or 70%, and preferably 80%, 90%, 95%, 100%, or 105% or more of human serum albumin or mouse serum albumin's ligand binding activity.

The albumin protein may include the amino acid sequence of bovine serum albumin. Bovine serum albumin proteins include any albumin having an amino acid sequence naturally occurring in cows, for example, as described by Swissprot accession number P02769, and variants thereof as defined herein. Bovine serum albumin proteins also includes fragments of full-length bovine serum albumin or variants thereof, as defined herein.

The albumin protein may comprise the sequence of an albumin derived from one of serum albumin from dog (e.g., Swissprot accession number P49822-1), pig (e.g., Swissprot accession number P08835-1), goat (e.g., Sigma product no. A2514 or A4164), cat (e.g., Swissprot accession number P49064-1), chicken (e.g., Swissprot accession number P19121-1), ovalbumin (e.g., chicken ovalbumin) (e.g., Swissprot accession number P01012-1), turkey ovalbumin (e.g., Swissprot accession number 073860-1), donkey (e.g., Swissprot accession number Q5XLE4-1), guinea pig (e.g., Swissprot accession number Q6WDN9-1), hamster (e.g., as described in DeMarco et al. International Journal for Parasitology 37(11): 1201-1208 (2007)), horse (e.g., Swissprot accession number P35747-1), rhesus monkey (e.g., Swissprot accession number Q28522-1), mouse (e.g., Swissprot accession number P07724-1), pigeon (e.g., as defined by Khan et al. Int. J. Biol. Macromol. 30(3-4), 171-8 (2002)), rabbit (e.g., Swissprot accession number P49065-1), rat (e.g., Swissprot accession number P02770-1) or sheep (e.g., Swissprot accession number P14639-1), and includes variants and fragments thereof as defined herein.

Many naturally-occurring mutant forms of albumin are known to those skilled in the art. Naturally-occurring mutant forms of albumin are described in, for example, Peters, et al. All About Albumin: Biochemistry, Genetics and Medical Applications, Academic Press, Inc., San Diego, Calif., p. 170-181 (1996).

Albumin proteins of the invention include variants of naturally-occurring albumin proteins. A variant albumin refers to an albumin protein having at least one amino acid mutation, such as an amino acid mutation generated by an insertion, deletion, or substitution, either conservative or non-conservative, provided that such changes result in an albumin protein for which at least one basic property has not been significantly altered (e.g., has not been altered by more than 5%, 10%, 15%, 20%, 25%, 30%, 35%, or 40%). Exemplary properties which may define the activity of an albumin protein include binding activity (e.g., including binding specificity or affinity to bilirubin, or a fatty acid such as a long-chain fatty acid), osmolarity, or behavior in a certain pH-range.

Typically an albumin protein variant will have at least 40%, at least 50%, at least 60%, and preferably at least 70%, at least 80%, at least 90%, at least 95%, at least 96%, at least 97%, at least 98%, or at least 99% amino acid sequence identity with a naturally-occurring albumin protein, such as the albumin protein described in WO 2020/051498, WO 2020/252393, or WO 2020/252396, each of which is hereby incorporated by reference.

Methods for the production and purification of recombinant human albumins are well-established (Sleep et al. Biotechnology, 8(1):42-6 (1990)), and include the production of recombinant human albumin for pharmaceutical applications (Bosse et al. J Clin Pharmacol 45(1):57-67 (2005)). The three-dimensional structure of HSA has been elucidated by X-ray crystallography (Carter et al. Science. 244(4909): 1195-8(1998)); Sugio et al. Protein Eng. 12(6):439-46 (1999)). The HSA polypeptide chain has 35 cysteine residues, which form 17 disulfide bonds, and one unpaired (e.g., free) cysteine at position 34 of the mature protein. Cys-34 of HSA has been used for conjugation of molecules to albumin (Leger et al. Bioorg Med Chem Lett 14(17):4395-8 (2004); Thibaudeau et al. Bioconjug Chem 16(4):1000-8 (2005)), and provides a site for site-specific conjugation.

### Conjugation of Albumin Proteins

An albumin protein of the invention may be conjugated to (e.g., by way of a covalent bond) to any therapeutic agent. An albumin protein may be conjugated to a compound (e.g., a dimer of zanamivir or an analog thereof) by the methods described herein.

An albumin protein of the invention may be conjugated to any compound of the invention by way of an amino acid located within 10 amino acid residues of the C-terminal or N-terminal end of the albumin protein. An albumin protein may include a C-terminal or N-terminal polypeptide fusion of 1, 2, 3, 4, 5, 6, 7, 8, 9, 10, 15, or 20 or more amino acid. The C-terminal or N-terminal polypeptide fusion may include one or more solvent-exposed cysteine or lysine residues, which may be used for covalent conjugation of a therapeutic agent.

Albumin proteins of the invention include any albumin protein which has been engineered to include one or more solvent-exposed cysteine or lysine residues, which may provide a site for conjugation to a compound of the invention. Most preferably, the albumin protein will contain a single solvent-exposed cysteine or lysine, thus enabling site-specific conjugation of a compound of the invention.

Exemplary methods for the production of engineered variants of albumin proteins that include one or more conjugation-competent cysteine residues are provided in U.S. Patent Application No. 2017/0081389, which is incorporated herein by reference in its entirety. Briefly, preferred albumin protein variants are those comprising a single, solvent-exposed, unpaired (e.g., free) cysteine residue, thus enabling site-specific conjugation of a linker to the cysteine residue.

Albumin proteins which have been engineered to enable chemical conjugation to a solvent-exposed, unpaired cysteine residue include the albumin protein described in WO 2020/051498, WO 2020/252393, or WO 2020/252396, each of which is hereby incorporated by reference.

In some embodiments of the invention, the net result of the substitution, deletion, addition, or insertion events of (a), (b), (c) and/or (d) is that the number of conjugation competent cysteine residues of the polypeptide sequence is increased relative to the parent albumin sequence. In some embodiments of the invention, the net result of the substitution, deletion, addition, or insertion events of (a), (b), (c) and/or (d) is that the number of conjugation competent-cysteine residues of the polypeptide sequence is one, thus enabling site-specific conjugation.

Preferred albumin protein variants also include albumin proteins having a single solvent-exposed lysine residue, thus enabling site-specific conjugation of a linker to the lysine residue. Such variants may be generated by engineering an albumin protein, including any of the methods previously described (e.g., insertion, deletion, substitution, or C-terminal or N-terminal fusion).

### V. Linkers

A linker refers to a linkage or connection between two or more components in a conjugate described herein (e.g., between two neuraminidase inhibitors in a conjugate described herein, between a neuraminidase inhibitor and an Fc domain in a conjugate described herein, and/or between a dimer of two neuraminidase inhibitors and an Fc domain in a conjugate described herein).

### Linkers in Conjugates Having an Fc Domain Covalently Linked to Dimers of Neuraminidase Inhibitors

In a conjugate containing an Fc domain monomer or an Fc domain covalently linked to one or more dimers of neuraminidase inhibitors as described herein, a linker in the conjugate (e.g., L or L′) may be a branched structure. As described further herein, a linker in a conjugate described herein (e.g., L or L′) may be a multivalent structure, e.g., a divalent or trivalent structure having two or three arms, respectively. In some embodiments when the linker has three arms, two of the arms may be attached to the first and second neuraminidase inhibitors and the third arm may be attached to the Fc domain monomer or an Fc domain. In some embodiments when the linker has two arms, one arm may be attached to an Fc domain and the other arm may be attached to one of the two neuraminidase inhibitors. In other embodiments, a linker with two arms may be used to attach the two neuraminidase inhibitors on a conjugate containing an Fc domain covalently linked to one or more dimers of neuraminidase inhibitors.

### Linking Groups

In some embodiments, a linker provides space, rigidity, and/or flexibility between the neuraminidase inhibitors and the Fc domain monomer or an Fc domain in the conjugates described here or between two neuraminidase inhibitors in the conjugates described herein. In some embodiments, a linker may be a bond, e.g., a covalent bond, e.g., an amide bond, a disulfide bond, a C—O bond, a C—N bond, a N—N bond, a C—S bond, or any kind of bond created from a chemical reaction, e.g., chemical conjugation. In some embodiments, a linker (e.g., L or L′ as shown in any one of formulas (M-I) or (D-I)-(D-VIII)) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). In some embodiments, a linker (L or L) includes no more than 250 non-hydrogen atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 non-hydrogen atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 non-hydrogen atom(s)). In some embodiments, the backbone of a linker (L or L) includes no more than 250 atoms (e.g., 1-2, 1-4, 1-6, 1-8, 1-10, 1-12, 1-14, 1-16, 1-18, 1-20, 1-25, 1-30, 1-35, 1-40, 1-45, 1-50, 1-55, 1-60, 1-65, 1-70, 1-75, 1-80, 1-85, 1-90, 1-95, 1-100, 1-110, 1-120, 1-130, 1-140, 1-150, 1-160, 1-170, 1-180, 1-190, 1-200, 1-210, 1-220, 1-230, 1-240, or 1-250 atom(s); 250, 240, 230, 220, 210, 200, 190, 180, 170, 160, 150, 140, 130, 120, 110, 100, 95, 90, 85, 80, 75, 70, 65, 60, 55, 50, 45, 40, 35, 30, 28, 26, 24, 22, 20, 18, 16, 14, 12, 10, 9, 8, 7, 6, 5, 4, 3, 2, or 1 atom(s)). The “backbone” of a linker refers to the atoms in the linker that together form the shortest path from one part of the conjugate to another part of the conjugate. The atoms in the backbone of the linker are directly involved in linking one part of the conjugate to another part of the conjugate. For examples, hydrogen atoms attached to carbons in the backbone of the linker are not considered as directly involved in linking one part of the conjugate to another part of the conjugate.

Molecules that may be used to make linkers (L or L′) include at least two functional groups, e.g., two carboxylic acid groups. In some embodiments of a trivalent linker, two arms of a linker may contain two dicarboxylic acids, in which the first carboxylic acid may form a covalent linkage with the first neuraminidase inhibitor in the conjugate and the second carboxylic acid may form a covalent linkage with the second neuraminidase inhibitor in the conjugate, and the third arm of the linker may for a covalent linkage (e.g., a C—O bond) with an Fc domain monomer or an Fc domain in the conjugate. In some embodiments of a divalent linker, the divalent linker may contain two carboxylic acids, in which the first carboxylic acid may form a covalent linkage with one component (e.g., a neuraminidase inhibitor) in the conjugate and the second carboxylic acid may form a covalent linkage (e.g., a C—S bond or a C—N bond) with another component (e.g., an Fc domain monomer or an Fc domain) in the conjugate.

In some embodiments, dicarboxylic acid molecules may be used as linkers (e.g., a dicarboxylic acid linker). For example, in a conjugate containing an Fc domain monomer or an Fc domain covalently linked to one or more dimers of neuraminidase inhibitors, the first carboxylic acid in a dicarboxylic acid molecule may form a covalent linkage with a hydroxyl or amine group of the first neuraminidase inhibitor and the second carboxylic acid may form a covalent linkage with a hydroxyl or amine group of the second neuraminidase inhibitor. In some embodiments, dicarboxylic acid molecules are further functionalized to contain one or more additional functional groups. Dicarboxylic acids may be further functionalized, for example, to provide an attachment point to an Fc domain monomer or an Fc domain (e.g., by way of a linker, such as a PEG linker).

In some embodiments, when the neuraminidase inhibitor is attached to Fc domain monomer or an Fc domain, the linking group may comprise a moiety comprising a carboxylic acid moiety and an amino moiety that are spaced by from 1 to 25 atoms. In some embodiments, a linking group may include a moiety including a carboxylic acid moiety and an amino moiety may be further functionalized to contain one or more additional functional groups. Such linking groups may be further functionalized, for example, to provide an attachment point to an Fc domain monomer or an Fc domain (e.g., by way of a linker, such as a PEG linker).

In some embodiments, when the neuraminidase inhibitor is attached to Fc domain monomer or an Fc domain, the linking group may comprise a moiety comprising two or amino moieties (e.g., a diamino moiety) that are spaced by from 1 to 25 atoms. In some embodiments, a linking group may include a diamino moiety may be further functionalized to contain one or more additional functional groups. Such diamino linking groups may be further functionalized, for example, to provide an attachment point to an Fc domain monomer or an Fc domain (e.g., by way of a linker, such as a PEG linker).

In some embodiments, a molecule containing an azide group may be used to form a linker, in which the azide group may undergo cycloaddition with an alkyne to form a 1,2,3-triazole linkage. In some embodiments, a molecule containing an alkyne group may be used to form a linker, in which the alkyne group may undergo cycloaddition with an azide to form a 1,2,3-triazole linkage. In some embodiments, a molecule containing a maleimide group may be used to form a linker, in which the maleimide group may react with a cysteine to form a C—S linkage. In some embodiments, a molecule containing one or more sulfonic acid groups may be used to form a linker, in which the sulfonic acid group may form a sulfonamide linkage with the linking nitrogen in a neuraminidase inhibitor. In some embodiments, a molecule containing one or more isocyanate groups may be used to form a linker, in which the isocyanate group may form a urea linkage with the linking nitrogen in a neuraminidase inhibitor. In some embodiments, a molecule containing one or more haloalkyl groups may be used to form a linker, in which the haloalkyl group may form a covalent linkage, e.g., C—N and C—O linkages, with a neuraminidase inhibitor.

In some embodiments, a linker (L or L′) may comprise a synthetic group derived from, e.g., a synthetic polymer (e.g., a polyethylene glycol (PEG) polymer). In some embodiments, a linker may comprise one or more amino acid residues. In some embodiments, a linker may be an amino acid sequence (e.g., a 1-25 amino acid, 1-10 amino acid, 1-9 amino acid, 1-8 amino acid, 1-7 amino acid, 1-6 amino acid, 1-5 amino acid, 1-4 amino acid, 1-3 amino acid, 1-2 amino acid, or 1 amino acid sequence). In some embodiments, a linker (L or L′) may include one or more optionally substituted C1-C20 alkylene, optionally substituted C1-C20 heteroalkylene (e.g., a PEG unit), optionally substituted C2-C20 alkenylene (e.g., C2 alkenylene), optionally substituted C2-C20 heteroalkenylene, optionally substituted C2-C20 alkynylene, optionally substituted C2-C20 heteroalkynylene, optionally substituted C3-C20 cycloalkylene (e.g., cyclopropylene, cyclobutylene), optionally substituted C3-C20 heterocycloalkylene, optionally substituted C4-C20 cycloalkenylene, optionally substituted C4-C20 heterocycloalkenylene, optionally substituted C8-C20 cycloalkynylene, optionally substituted C8-C20 heterocycloalkynylene, optionally substituted C5-C15 arylene (e.g., C6 arylene), optionally substituted C2-C15 heteroarylene (e.g., imidazole, pyridine), O, S, NRⁱ (Rⁱ is H, optionally substituted C1-C20 alkyl, optionally substituted C1-C20 heteroalkyl, optionally substituted C2-C20 alkenyl, optionally substituted C2-C20 heteroalkenyl, optionally substituted C2-C20 alkynyl, optionally substituted C2-C20 heteroalkynyl, optionally substituted C3-C20 cycloalkyl, optionally substituted C3-C20 heterocycloalkyl, optionally substituted C4-C20 cycloalkenyl, optionally substituted C4-C20 heterocycloalkenyl, optionally substituted C8-C20 cycloalkynyl, optionally substituted C8-C20 heterocycloalkynyl, optionally substituted C5-C15 aryl, or optionally substituted C2-C15 heteroaryl), P, carbonyl, thiocarbonyl, sulfonyl, phosphate, phosphoryl, or imino.

### Conjugation Chemistries

Neuraminidase inhibitor dimers (e.g., in a conjugate of any one of formulas (M-I) or (D-I)-(D-VIII)) may be conjugated to an Fc domain monomer or an Fc domain, e.g., by way of a linker, by the methods described herein. The following conjugation chemistries can be contemplated, e.g., for conjugation of a PEG linker (e.g., a functionalized PEG linker) to an Fc domain monomer or an Fc domain.

For example, in the methods disclosed herein, E (e.g., Fc domain monomer or an albumin protein (e.g., by way of a linker)) is reacted with intermediate compounds of formula (DF-I) or (DF-II), which are functionalized with a phenyl ester group (e.g., a trifluorophenyl ester group or a tetrafluorophenyl ester group). Conjugation (e.g., by acylation) of E and the intermediate compound of formula (DF-I) or (DF-II) forms a conjugate (e.g., a conjugate described by formula (D-I)).

Intermediate compounds of formula (DF-I) or (DF-II) can be synthesized by reacting a phenol (e.g., tetrafluorophenol or trifluorophenol) with a compound including

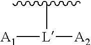

and a linker including an activated carboxylic acid.

Intermediate compounds of formula (DF-I) or (DF-II) can also be synthesized by reacting a compound (e.g., a compound of formula (D-G3-A)) comprising a functional group (e.g., G^{a}), a linker (e.g., L″), and a phenyl ester (e.g., trifluorophenyl ester or tetrafluorophenyl ester) with a compound (e.g., a compound of formula (D-G3-B) comprising a functional group (e.g., G^{b}) and

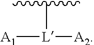

Reaction of two or more components in an intermediate compound (e.g., a compound of formula (DF-I) or (DF-II)) may be accomplished using well-known organic chemical synthesis techniques and methods. Complementary functional groups (e.g., G^{a} and G^{b}) on two components may react with each other to form a covalent bond. Complementary functional groups (e.g., G^{a} and G^{b}) on two components may react with each other to form a chemical moiety, e.g., G. Examples of complementary reactive functional groups include, but are not limited to, e.g., maleimide and cysteine, amine and activated carboxylic acid (e.g., to form an amide linkage), thiol and maleimide, activated sulfonic acid and amine, isocyanate and amine, azide and alkyne (e.g., click chemistry to form a triazole), and alkene and tetrazine.

Other examples of functional groups capable of reacting with amino groups include, e.g., alkylating and acylating agents. Representative alkylating agents include: (i) an α-haloacetyl group, e.g., XCH₂CO— (where X═Br, Cl, or I); (ii) a N-maleimide group, which may react with amino groups either through a Michael type reaction or through acylation by addition to the ring carbonyl group; (iii) an aryl halide, e.g., a nitrohaloaromatic group; (iv) an alkyl halide; (v) an aldehyde or ketone capable of Schiff's base formation with amino groups; (vi) an epoxide, e.g., an epichlorohydrin and a bisoxirane, which may react with amino, sulfhydryl, or phenolic hydroxyl groups; (vii) a chlorine-containing of s-triazine, which is reactive towards nucleophiles such as amino, sulfhydryl, and hydroxyl groups; (viii) an aziridine, which is reactive towards nucleophiles such as amino groups by ring opening; (ix) a squaric acid diethyl ester; and (x) an α-haloalkyl ether.

Examples of amino-reactive acylating groups include, e.g., (i) an isocyanate and an isothiocyanate; (ii) a sulfonyl chloride; (iii) an acid halide; (iv) an active ester, e.g., a nitrophenylester or N-hydroxysuccinimidyl ester; (v) an acid anhydride, e.g., a mixed, symmetrical, or N-carboxyanhydride; (vi) an acylazide; and (vii) an imidoester. Aldehydes and ketones may be reacted with amines to form Schiffs bases, which may be stabilized through reductive amination.

It will be appreciated that certain functional groups may be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

In some embodiments, the intermediate compound (e.g., a compound of formula (DF-I) or (DF-II)) is synthesized via click chemistry (e.g., where G^{a} of formula (D-G3-A) is an azido group and G^{b} of formula (D-G3-B) is an alkynyl group; or where G^{a} of formula (D-G3-A) is an alknyl group and G^{b} of formula (D-G3-B) is an azido group). In some embodiments, the click chemistry includes the use of a Cu(I) source.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Tetrafluorophenyl Ester Conjugation

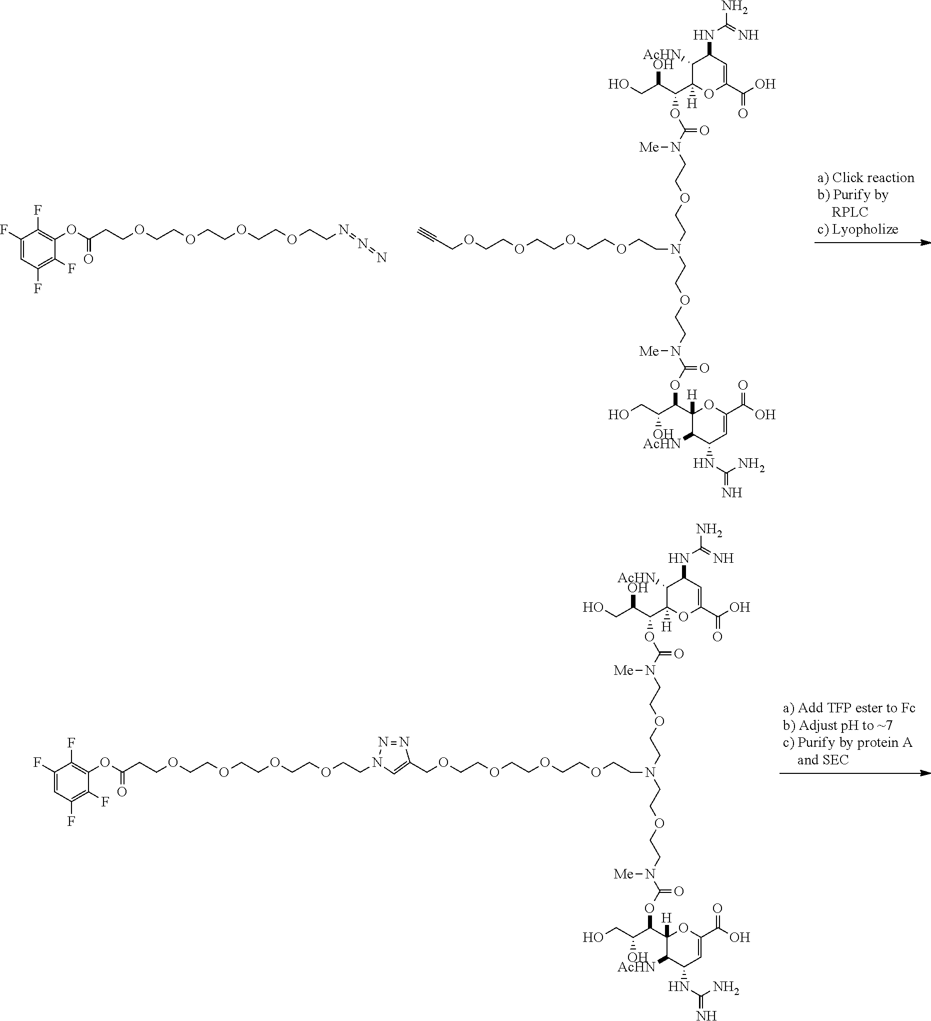

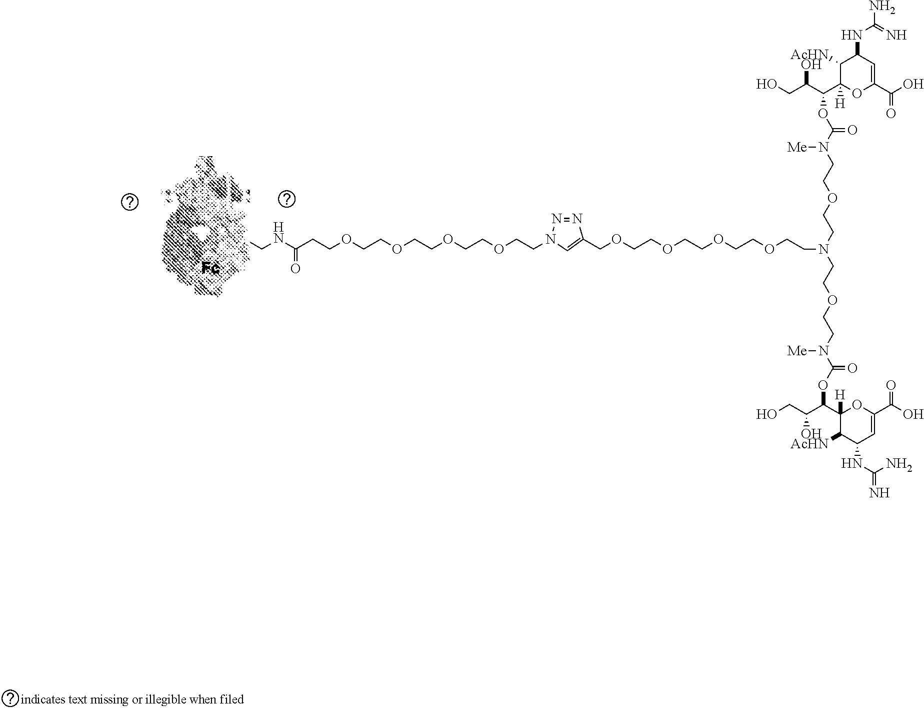

### Step a. Synthesis of Int-93

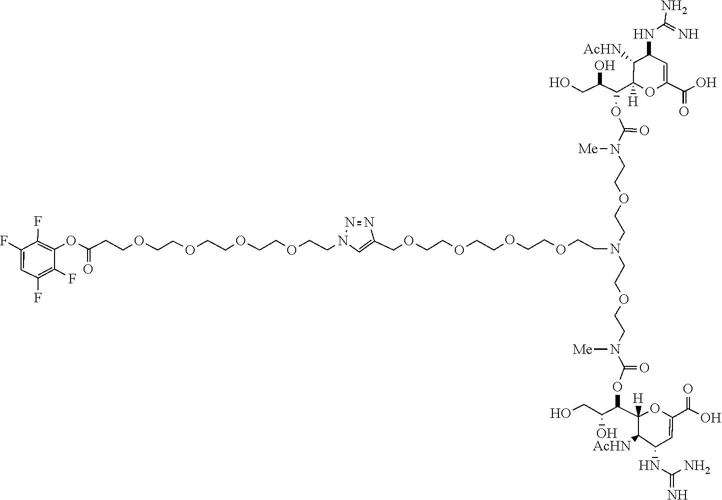

A solution of azido-PEG4-TFP ester (0.1 g, 0.067 mmol) and alkyne functionalized dimer (0.0383 g, 0.0871 mmol) in DMF (2.0 mL), were treated with a solution of copper(II)sulfate (0.0027 g, 0.0168 mmol), sodium ascorbate (0.0133 g, 0.067 mmol), and THPTA (0.0116 g, 0.027 mmol) at room temperature, in water (1.5 mL). The reaction was then vacuum flushed with nitrogen 3× and stirred under an atmosphere of nitrogen. LCMS after 30 min shows complete consumption of starting material. The reaction was acidified with 400 μL of acetic acid, and then purified directly by reverse phase chromatography eluting with a gradient of 5% to 100% acetonitrile/water with 0.1% TFA. The product containing fractions were combined, frozen, and lyophilized overnight. Yield of triple TFA salt was 69%. Ion(s) found by LCMS: (M+2H)⁺²=795.4, (M+3H)⁺³=530.8, (M+4H)⁺⁴=398.4.

### Step b

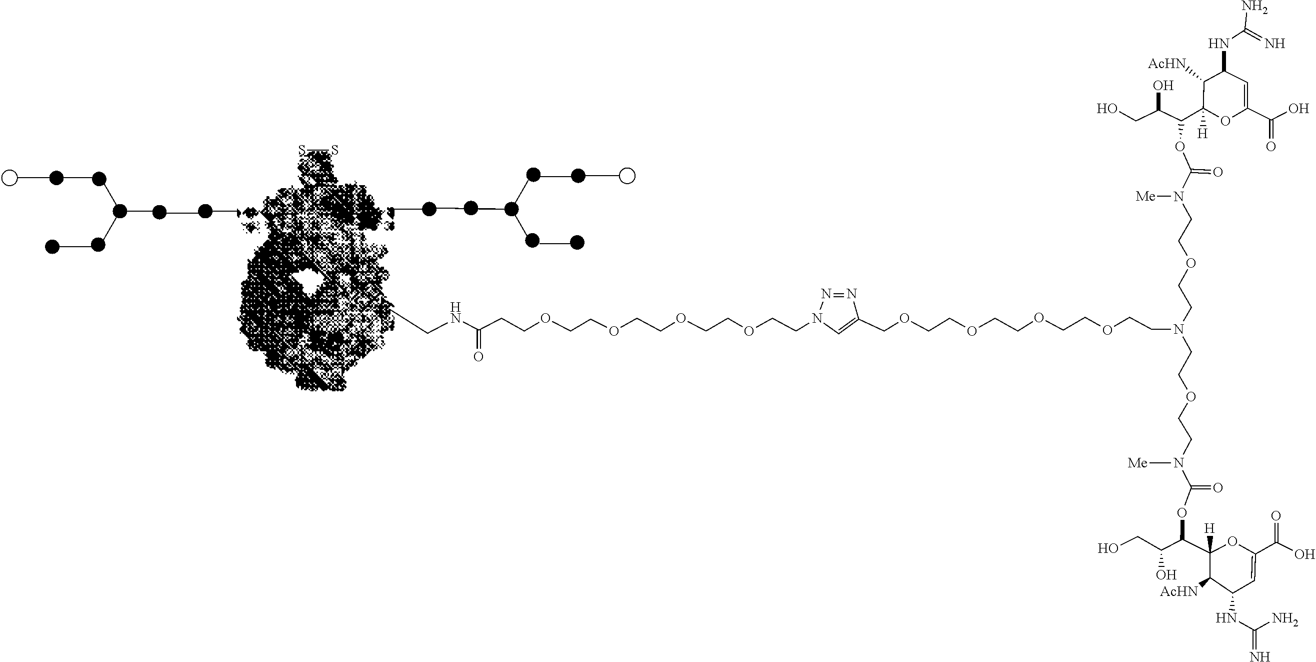

A solution of Fc (0.100 g in 5.2 mL, 1.717 μmol, MW=58,218, SEQ ID NO: 11) in pH=7.4 PBS buffer was treated with solid TFP ester (0.0273 g, 17.17 μmol) from the previous step. The pH was adjusted to ˜7.0 with borate buffer (120 μL, 1 M, pH 8.5) then was gently rocked at room temperature. Maldi TOE after 1.5 hr shows an average OAR of 3.3, which did not change upon further mixing. After 24 hr additional TEP ester (0.0073 g, 4.6 μmol) was added and rocking was continued for another 3 h. The crude conjugate was purified Protein A and SEC according to general purification methods. Total yield after Protein A was ˜83%, and after SEC ˜77%. Maldi TOE of the purified conjugate showed an average mass of 63,574, which equates to an average OAR of 4.0.

The synthesis described in Example 1 is advantageous at it avoids exposing the Fc to copper+2 and sodium ascorbate (for example, as described in described in WO 2020/051498 and WO 2021/046549), leading to a cleaner crude conjugate that is 98.9% pure by analytical SEC after protein A purification alone. At this level of purity it may be possible to eliminate the SEC purification which is time very consuming and costly. Initial by attempts with an azido-PEG4-NHS ester were only partially successful because the NHS ester is too reactive to be purified, and the crude click reaction mixture had to be mixed with the Fc, thus necessitating copper removal and high molecular weight aggregate removal (from exposure to sodium ascorbate). Also this approach did not generate OAR's greater than 2. Subsequent attempts using a less reactive active ester (TEP tetrafluorophenol) that is stable enough to withstand reverse phase purification and lyophilization, allows the click reaction with azido TEP ester to be done separate from the Fc, purified, and then mixed with the Fc. An average OAR of 4.0 was achieved and higher DARs are possible by adding more of the TEP ester.

The nucleic acid construct encoding the Fc included a nucleic acid encoding the amino acid sequence of SEQ ID NO: 4, which includes a C-terminal lysine residue. Upon expression, the C-terminal lysine of the Fc is proteolytically cleaved, resulting in an Fc having the sequence of SEQ ID NO: 11. The presence or absence of a C-terminal lysine does not alter the properties of the Fc or the corresponding conjugate.

The synthetic intermediate prior to the click conjugation (e.g., the zanamivir dimer conjugated to an alkyne-functionalized linker) was produced as described in WO 2020/051498 or WO 2021/046549, each of which is hereby incorporated by reference.

### Example 2. Trifluorophenyl Ester Conjugation

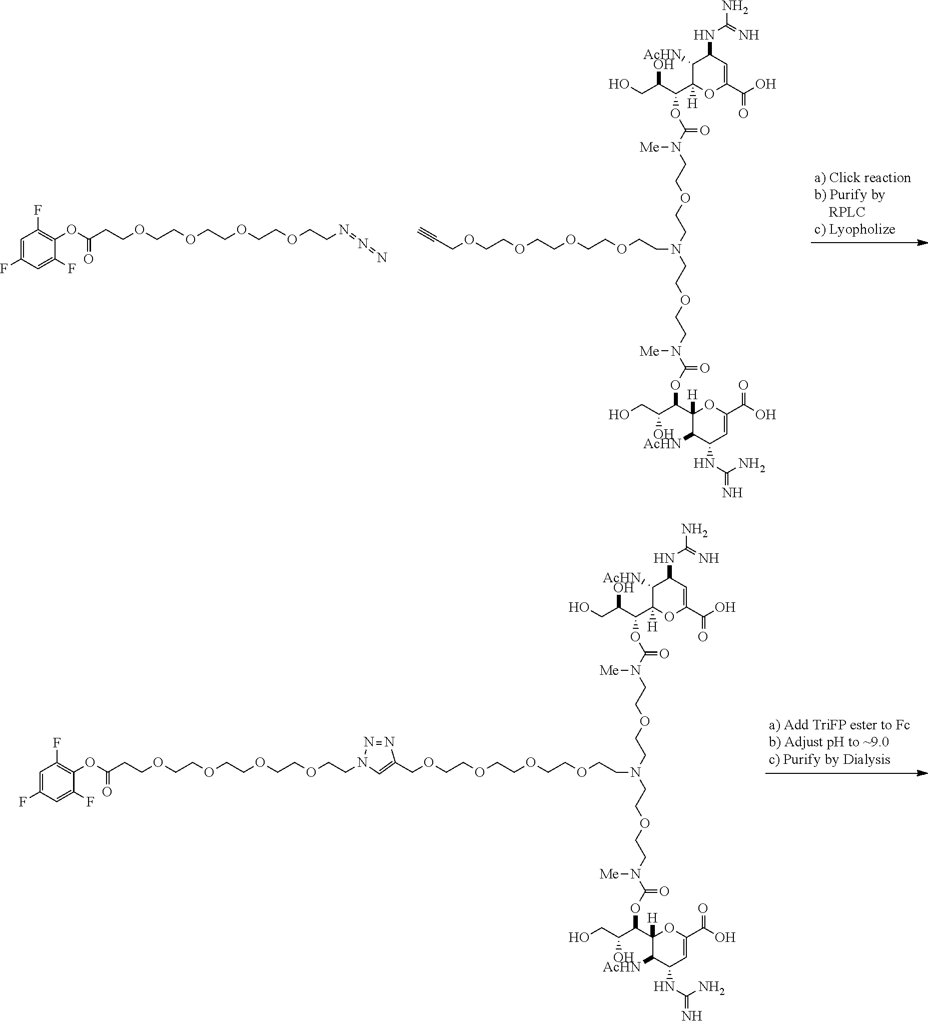

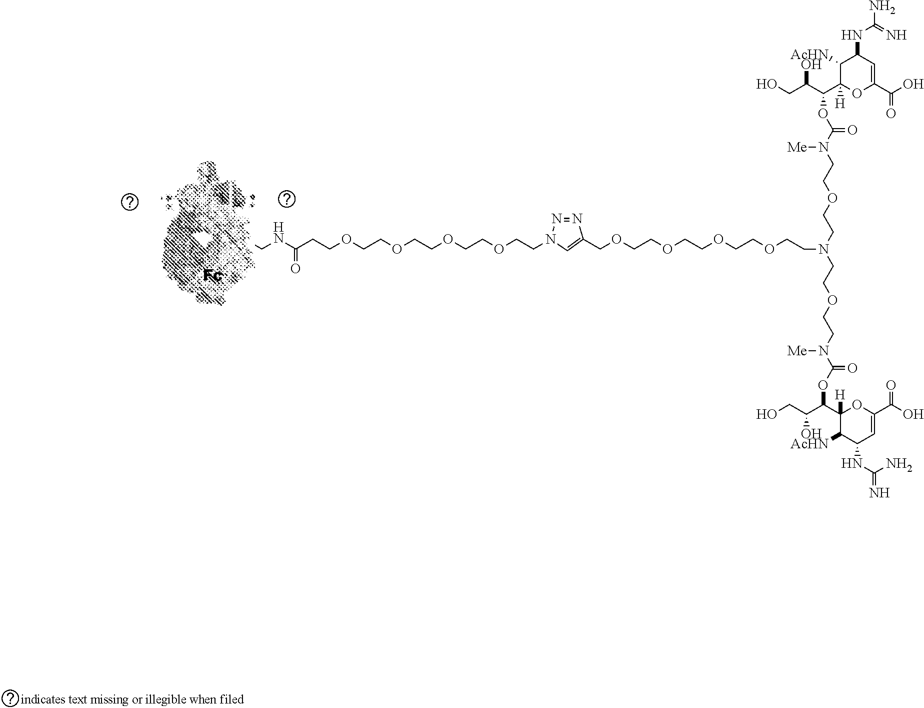

### Step a. Synthesis of Int-94

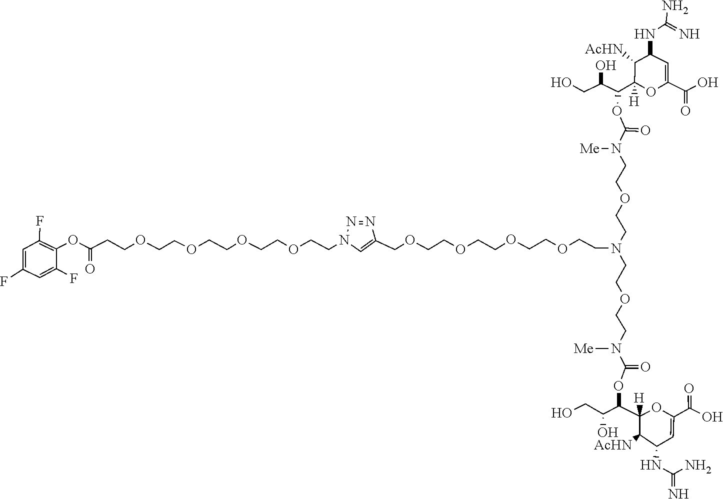

A solution of azido-PEG4-TriFP ester (0.405 g, 0.96 mmol) and alkyne functionalized dimer (Int-83, 0.850 g, 0.74 mmol) in DMF (4.0 mL), were cooled to 0° C. To this solution was added a solution of copper(II)sulfate (0.030 g, 0.18 mmol) and sodium ascorbate (0.146 g, 0.74 mmol), in water (4.0 mL). The reaction was then vacuum flushed with nitrogen 3× and stirred under an atmosphere of nitrogen. LCMS after 30 min shows complete consumption of starting material. The reaction was acidified with acetic acid (0.1 mL, 1.75 mmol), and then purified directly by reversed phase chromatography eluting with a gradient of 0% to 80% acetonitrile/water with 0.1% TFA. The product containing fractions were combined frozen, and lyophilized. Yield of triple TFA salt was 65%, 920 mg. Ion(s) found by LCMS: (M+2H)⁺²=786.4, (M+3H)⁺³=524.8, (M+4H)⁺⁴=393.8.

### Step b

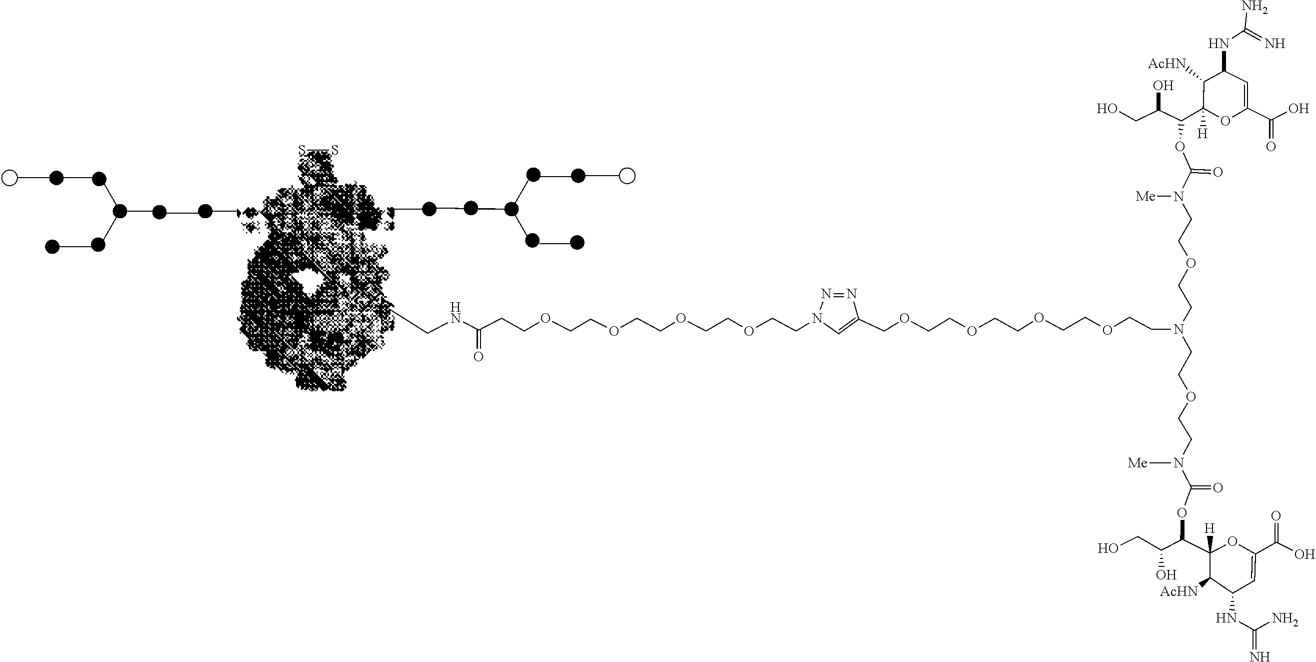

A solution of polypeptide having sequence of SEQ ID NO: 13 (2.0 g in 100 mL, 0.034 mmol, MW=58,200,YTE) in acetate buffer at pH 5.0 was treated with carbonate buffer (pH 9.5, 0.1 M, 24-30 mL) to adjust the requisite pH to 9.0. Solid TFP ester (0.710 g, 0.39 mmol) from the previous step was then added at which point the pH decreased back to 6.0-7.0. The pH was again adjusted to ˜9.0-9.5 with the carbonate buffer (12-18 mL). The solution was then gently rocked at room temperature for 3 h. Maldi TOF after 1.5 hr shows an average DAR of 3.5-4.0. After an additional 1 h the DAR had risen to 4.4-4.6 and the reaction was quenched with the addition of concentrated NH₄OH (0.100 mL). The crude conjugate was dialyzed with the following buffer: 120 mM NaCl, 250 mM Arginine, 0.1% sucrose pH 6 buffer. Total yield was ˜80%. Maldi TOF of the purified conjugate showed an average mass of 64,724, which equates to an average DAR of 4.6.

Trifluorophenyl ester compounds (e.g., the compound resulting from step a of this example or compounds of formula (F-I), (F-II), (F-II-A), (F-II-B), (G1-A), and (G2-A)) provide further advantages in the synthesis of protein-drug conjugates. For example, trifluorophenyl ester compounds exhibit increased stability, which allows for, e.g., purification by reverse phase chromatography and lyophilization with minimal hydrolysis of the activated ester.

The synthetic intermediate prior to the click conjugation (e.g., the zanamivir dimer conjugated to an alkyne-functionalized linker) was produced as described in WO 2020/051498 or WO 2021/046549, each of which is hereby incorporated by reference.

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. All publications, patents, and patent applications mentioned in the above specification are hereby incorporated by reference to the same extent as if each individual publication, patent or patent application was specifically and individually indicated to be incorporated by reference in its entirety.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

1. A method of synthesizing a conjugate of formula (M-I) or (D-I): 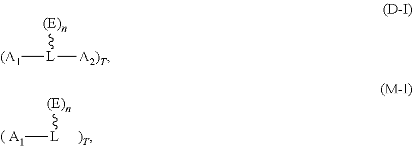
wherein A₁ and A₂ are each, independently, an anti-influenza moiety;
n is 1 or 2;
each E includes an Fc domain monomer or an albumin protein;
L is a linker covalently attached to E and to A₁ or A₁ and A₂;
T is an integer from 1 to 20; and
each squiggly line in formula (M-I) or (D-I) indicates that L is covalently attached to each E,
the method comprising the steps of:
(a) providing a first composition comprising E;
(b) providing a second composition comprising a compound of formula (DF-I), (MF-I), or a salt thereof: 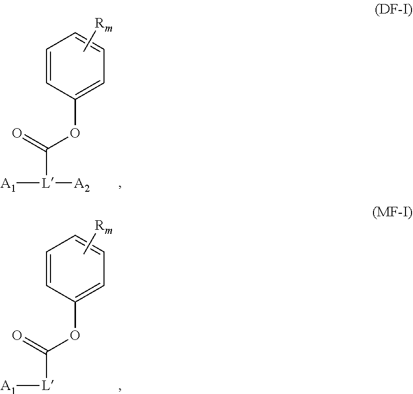
wherein
L′ is the remainder of L;
m is 0, 1, 2, 3, or 4; and
each R is, independently, halo, cyano, nitro, optionally substituted C₁-C₆ alkyl group, or optionally substituted C₁-C₆ heteroalkyl group; and
(c) combining the first composition, the second composition, and a buffer to form a mixture.

2. The method of claim 1, wherein each anti-influenza moiety is a small molecule.

3. The method of claim 1 or 2, wherein each anti-influenza molecule is selected from pimovidir, oseltamivir, zanamivir, sulfozanamivir, peramivir, Ianinamivir, amantadine, rimantadine, baloxavir, or an analog thereof.

4. The method of claim 1, wherein each A₁ and each A₂ is independently selected from any one of formulas (A-I)-(A-XIII): 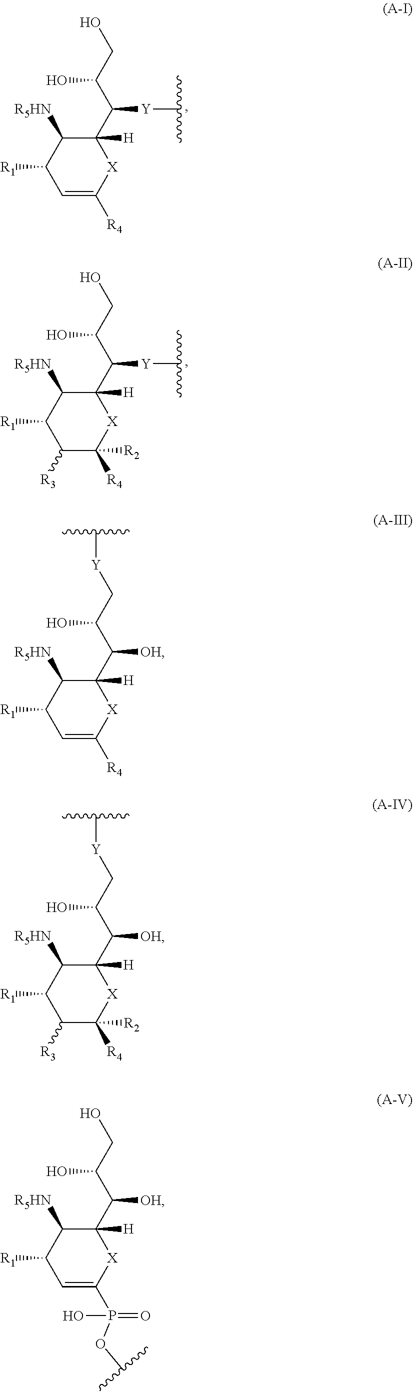 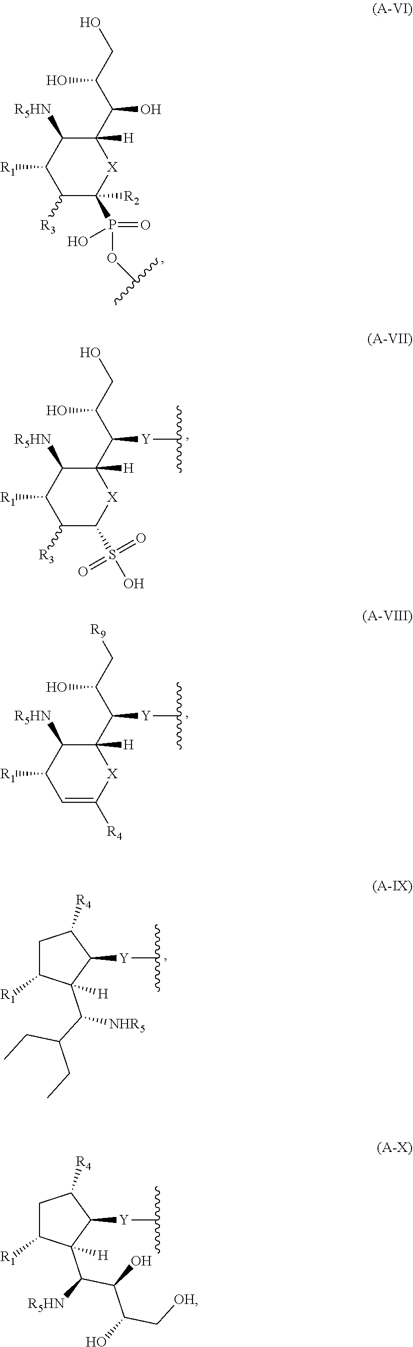 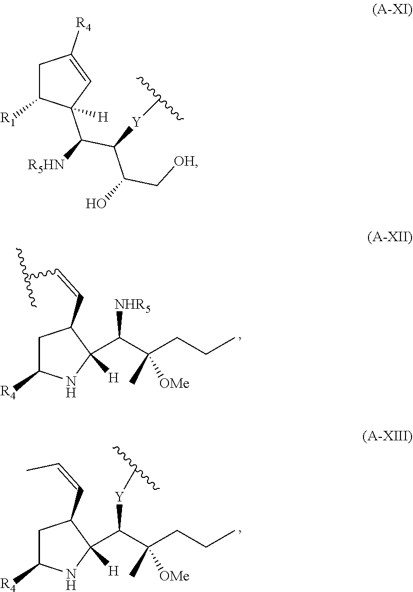
wherein R₁ is selected from —OH, —NH₂, —NHC(═NH)NH₂, and —NHC(═NH)NHR₆;
R₂ and R₃ are each independently selected from —H, —OH, —F, —Cl, and —Br;
R₄ is selected from —CO₂H, —P(═O)(OH)₂, —SO₃H;
R₅ is selected from —COCH₃, —COCF₃, —SO₂CH₃;
X is selected from —O— and —S—;
Y is selected from: 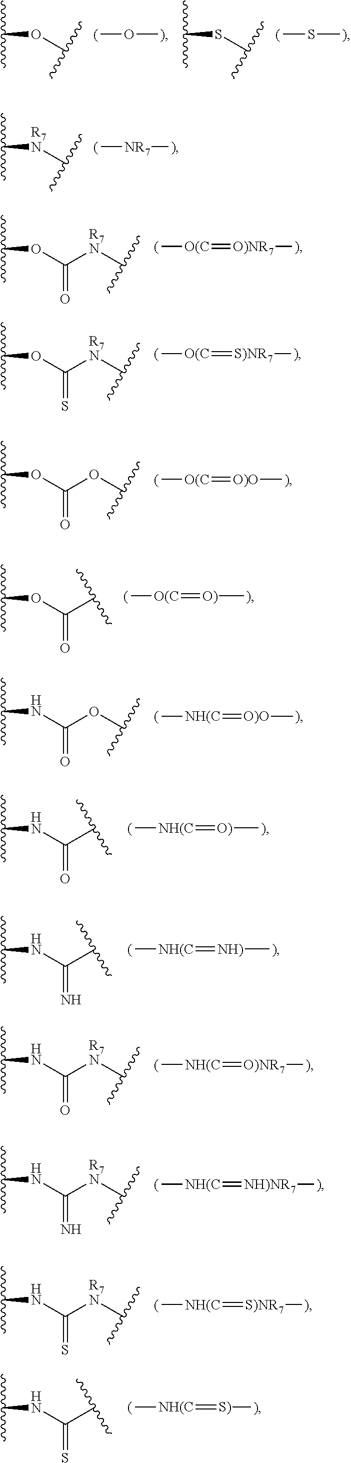 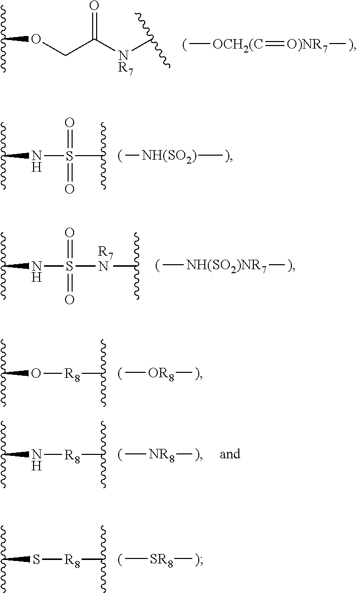
R₆ is selected from 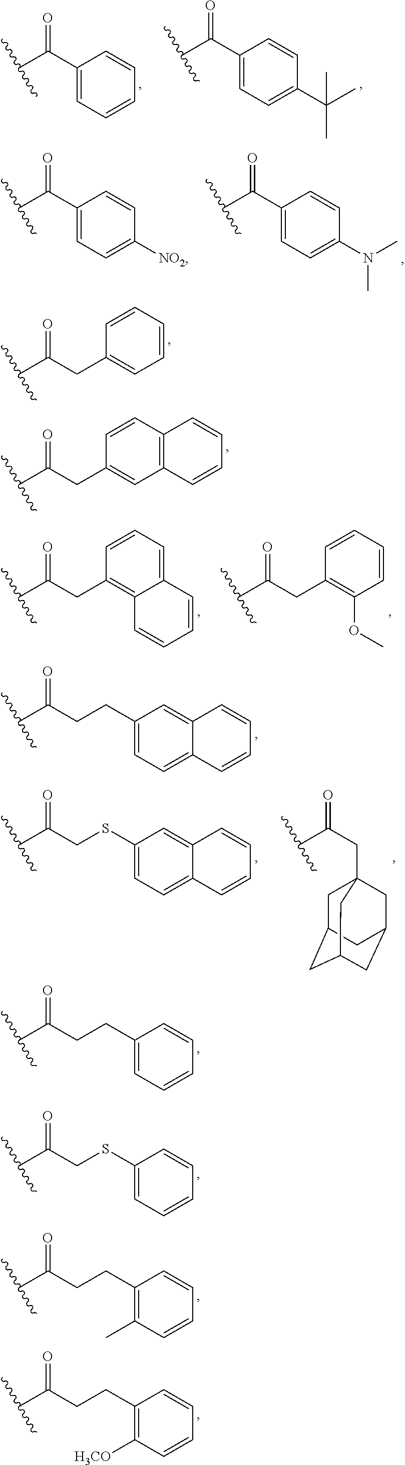 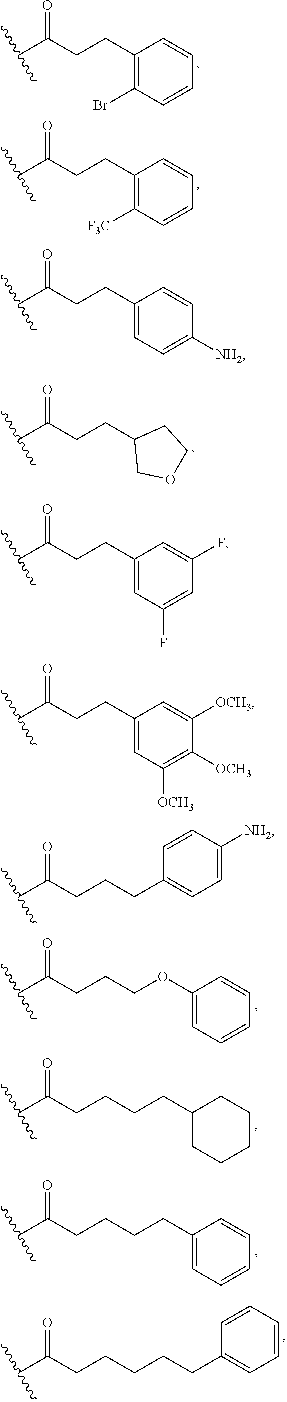 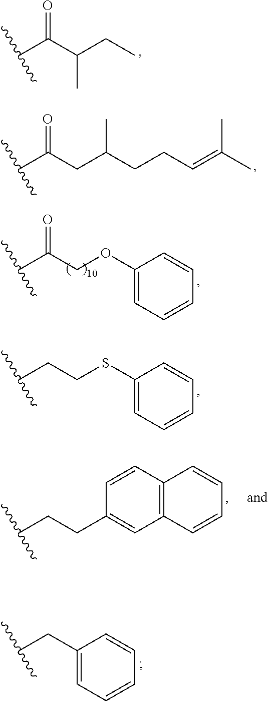
R₇ is selected from H, C1-C20 alkyl, C3-C20 cycloalkyl, C3-C20 heterocycloalkyl; C5-C15 aryl, and C2-C15 heteroaryl;
R₈ is selected from C3-C20 heterocycloalkyl, C5-C15 aryl, and C2-C15 heteroaryl;
R₉ is selected from —H, a halogen (e.g., Cl or F), —OR₁₀, —NHC(═O)R₇, optionally substituted C1-C20 alkyl, C3-C20 cycloalkyl, C3-C20 heterocycloalkyl; C5-C15 aryl, and C2-C15 heteroaryl; and
R₁₀ is selected from C1-C20 alkyl, C3-C20 cycloalkyl, C3-C20 heterocycloalkyl; C5-C15 aryl, and C2-C15 heteroaryl;
n is 1 or 2;
each E comprises an Fc domain monomer or an albumin protein;
L is a linker;
T is an integer from 1 to 20; and
each squiggly line indicates that L is covalently attached to each E.

5. A method of synthesizing a conjugate of formula (M-I) or (D-I): 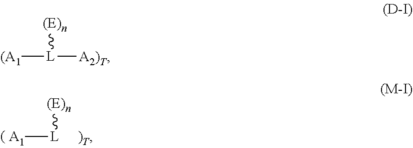
or a pharmaceutically acceptable salt thereof,
wherein each A₁ and each A₂ is independently selected from any one of formulas (A-I)-(A-XIII): 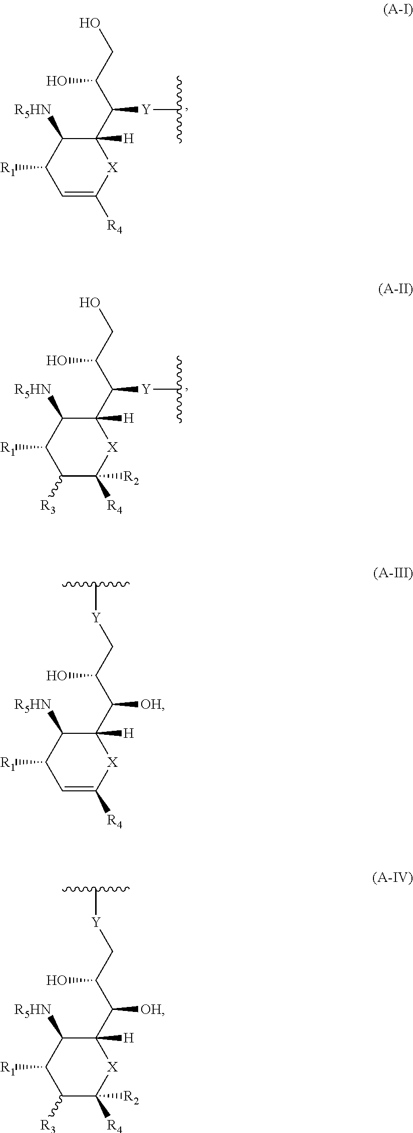 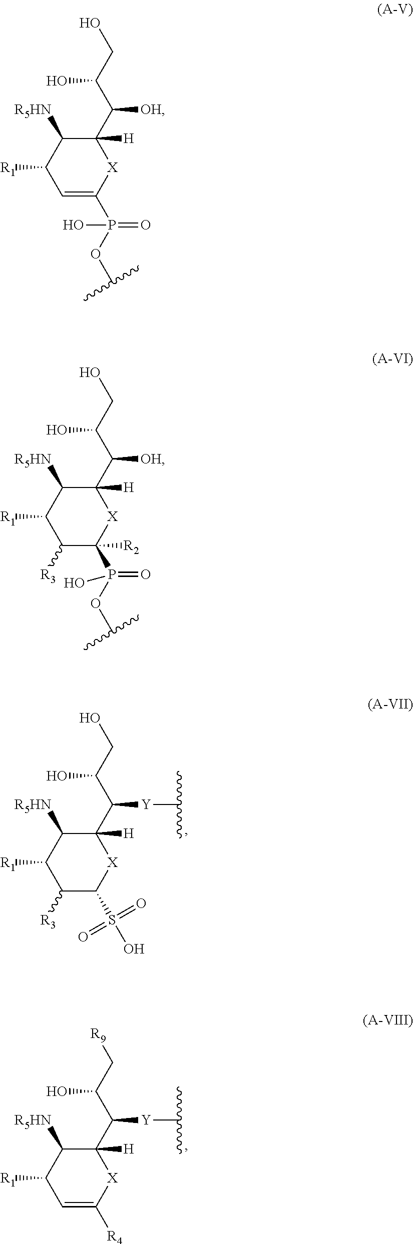 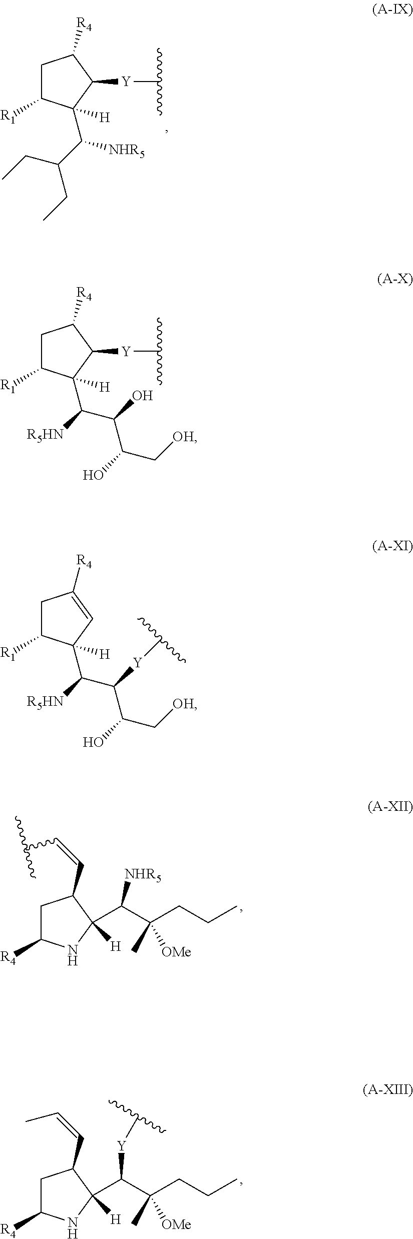
wherein R₁ is selected from —OH, —NH₂, —NHC(═NH)NH₂, and —NHC(═NH)NHR₆;
R₂ and R₃ are each independently selected from —H, —OH, —F, —Cl, and —Br;
R₄ is selected from —CO₂H, —P(═O)(OH)₂, —SO₃H;
R₅ is selected from —COCH₃, —COCF₃, —SO₂CH₃;
X is selected from —O— and —S—;
Y is selected from: 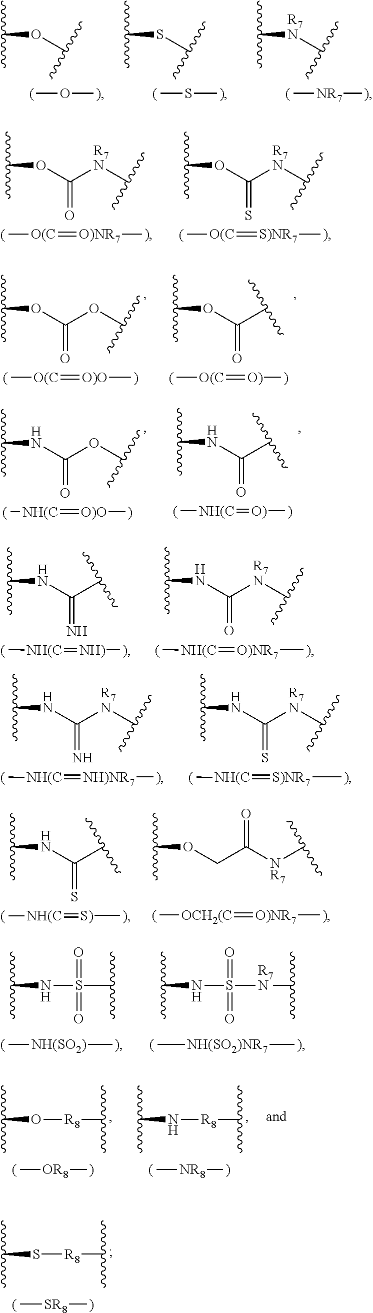
R₆ is selected from 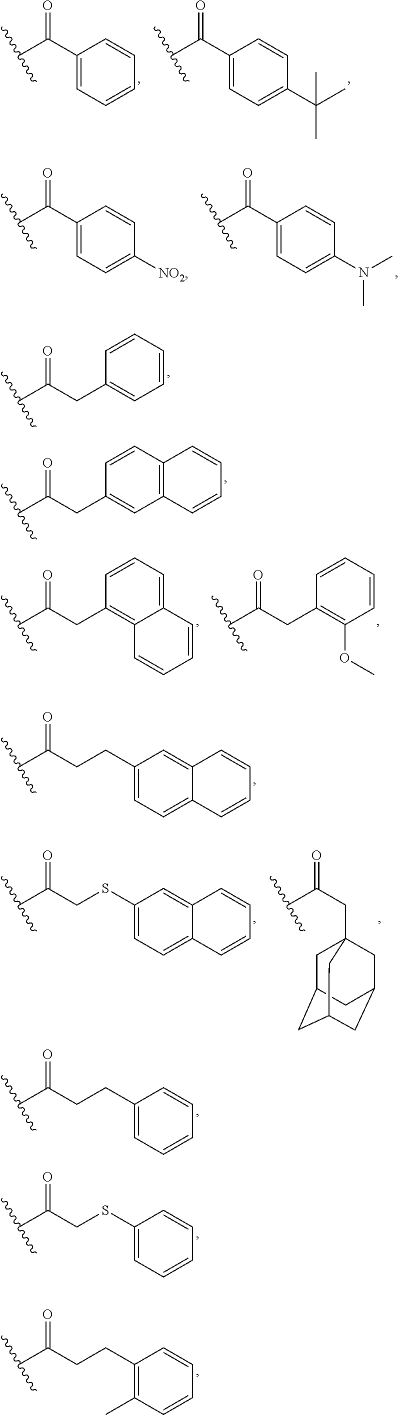 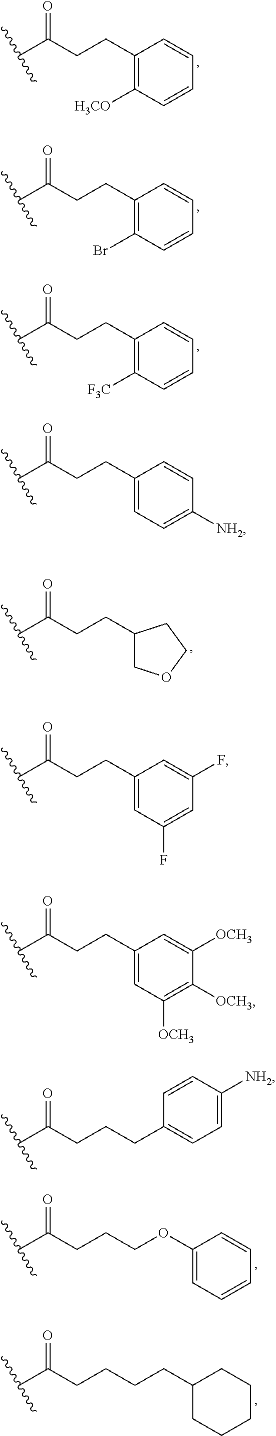 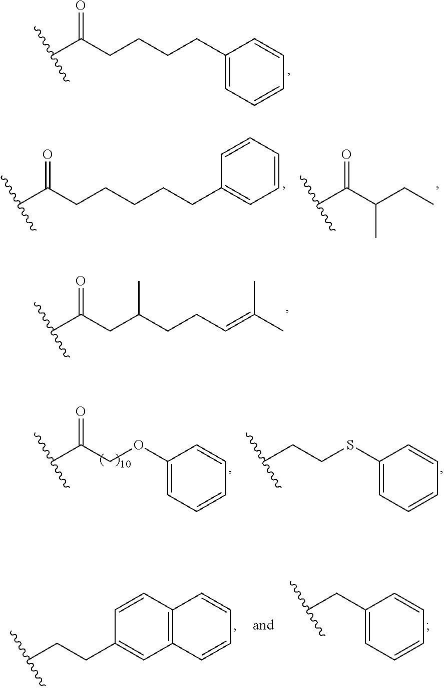
R₇ is selected from H, C1-C20 alkyl, C3-C20 cycloalkyl, C3-C20 heterocycloalkyl; C5-C15 aryl, and C2-C15 heteroaryl;
R₈ is selected from C3-C20 heterocycloalkyl, C5-C15 aryl, and C2-C15 heteroaryl;
R₉ is selected from —H, a halogen (e.g., Cl or F), —OR₁₀, —NHC(═O)R₇, optionally substituted C1-C20 alkyl, C3-C20 cycloalkyl, C3-C20 heterocycloalkyl; C5-C15 aryl, and C2-C15 heteroaryl; and
R₁₀ is selected from C1-C20 alkyl, C3-C20 cycloalkyl, C3-C20 heterocycloalkyl; C5-C15 aryl, and C2-C15 heteroaryl;
n is 1 or 2;
each E comprises an Fc domain monomer or an albumin protein;
L is a linker;
T is an integer from 1 to 20; and
each squiggly line indicates that L is covalently attached to each E,
the method comprising:
(a) providing a first composition comprising E;
(b) providing a second composition comprising a compound of formula (DF-II), (MF-II), or salt thereof: 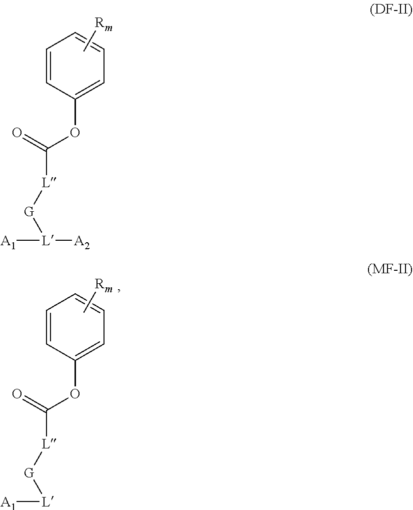
wherein
G is optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted C₃-C₁₀ cycloalkylene, optionally substituted C₂-C₁₀ heterocycloalkylene, optionally substituted C₆-C₁₀ arylene, or optionally substituted C₂-C₁₀ heteroarylene;
L′-G-L″ is the remainder of L;
m is 0, 1, 2, 3, or 4; and
each R is, independently, halo, cyano, nitro, optionally substituted C₁-C₆ alkyl group, or optionally substituted C₁-C₆ heteroalkyl group;
and
(c) combining the first composition, the second composition, and a buffer to form a mixture.

6. The method of any one of claims 1-5, wherein each R is halo.

7. The method of claim 6, wherein each R is, independently, F, Cl, Br, or I.

8. The method of claim 7, wherein each R is F.

9. The method of any one of claims 1-8, wherein m is 1, 2, 3, 4, or 5.

10. The method of claim 9, wherein m is 3 or 4.

11. The method of any one of claims 1-10, wherein m is 3.

12. The method of claim 11, wherein 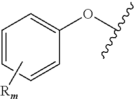
is

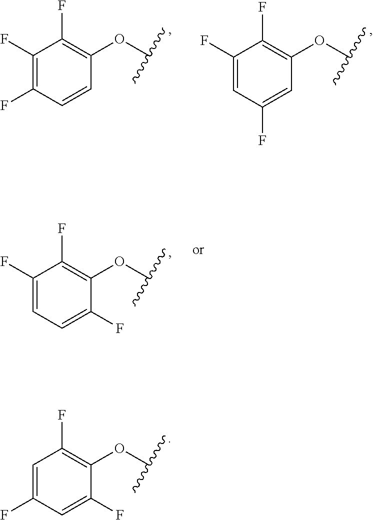 [SMILES: *Oc1ccc(F)c(F)c1F ; *Oc1cc(F)cc(F)c1F ; *Oc1c(F)ccc(F)c1F ; *Oc1c(F)cc(F)cc1F]

13. The method of claim 12, wherein 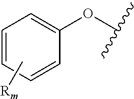
is

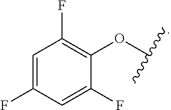 [SMILES: *Oc1c(F)cc(F)cc1F]

14. The method of any one of claims 1-10, wherein m is 4.

15. The method of claim 14, wherein

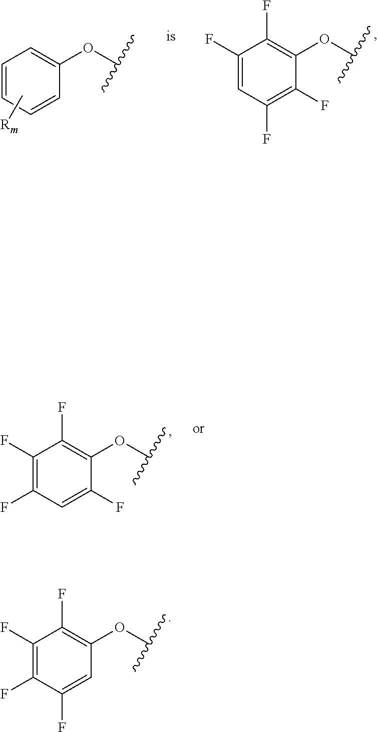

16. The method of claim 15, wherein 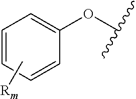
is

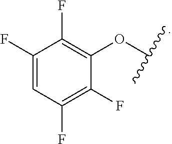 [SMILES: *Oc1c(F)c(F)cc(F)c1F]

17. The method of any one of claims 1-10, wherein 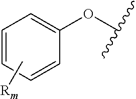
is

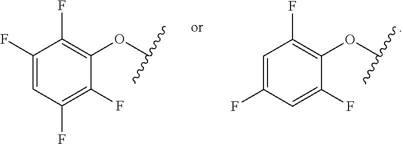 [SMILES: *Oc1c(F)c(F)cc(F)c1F ; *Oc1c(F)cc(F)cc1F]

18. The method of claim 17, wherein 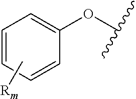
is

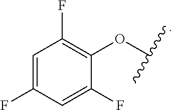 [SMILES: *Oc1c(F)cc(F)cc1F]

19. The method of claim 17, wherein 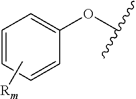
is

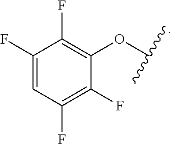 [SMILES: *Oc1c(F)c(F)cc(F)c1F]

20. The method of any one of claims 1-19, wherein the compound of (D-FI) or (DF-II) has the structure:

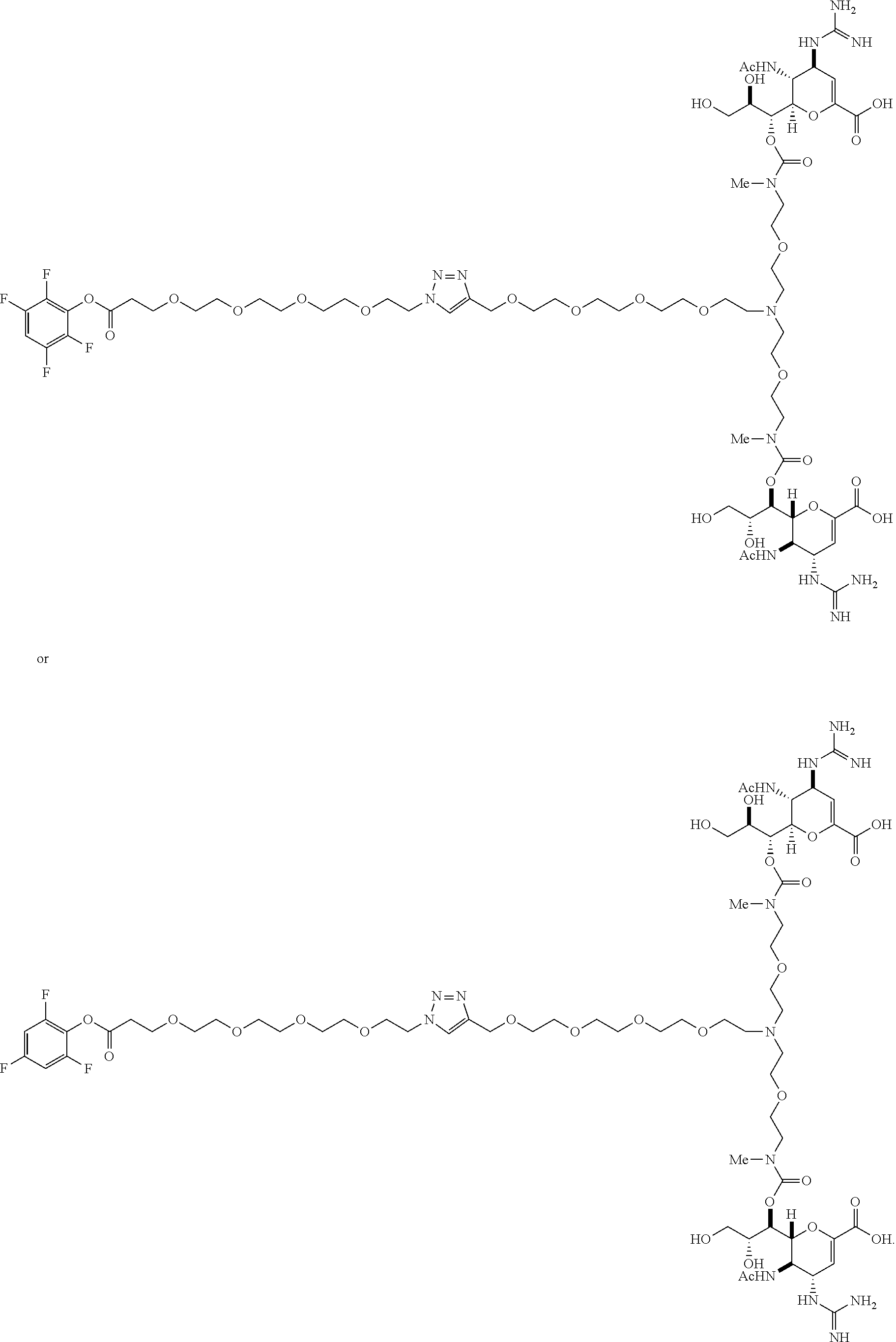

21. The method of any one of claims 1 to 20, wherein the buffer comprises borate or carbonate.

22. The method of any one of claims 1 to 21, wherein the buffer has a pH of about 7.0 to 10.0.

23. The method of claim 22, wherein the buffer has a pH of about 7.5 to 9.5.

24. The method of claim 22 or 23, wherein the buffer has a pH of about 7.5.

25. The method of claim 22 or 23, wherein the buffer has a pH of about 8.5.

26. The method of claim 22 or 23, wherein the buffer has a pH of about 9.5.

27. The method of any one of claims 1 to 26, wherein step (c) is conducted at a temperature of 20 to 30° C.

28. The method of claim 27, wherein step (c) is conducted at a temperature of 22 to 27° C.

29. The method of claim 27, wherein step (c) is conducted at a temperature of about 25° C.

30. The method of any one of claims 1 to 29, wherein step (c) is conducted for 2 to 12 hours.

31. The method of claim 30, wherein step (c) is conducted for about 2 hours.

32. The method of any one of claims 1 to 31, wherein the first composition comprises phosphate-buffered saline buffer.

33. The method of any one of claims 1 to 32, wherein the buffer has a pH of about 7.0 to 8.0.

34. The method of claim 33, wherein the buffer has a pH of about 7.5.

35. The method of any one of claims 1 to 34, wherein the second composition comprises DMF.

36. The method of any one of claims 1 to 35, wherein the method further comprises a purification step.

37. The method of claim 36, wherein the purification step comprises dialysis in arginine buffer.

38. The method of claim 36 or 37, wherein the purification step comprises a buffer exchange.

39. A method of synthesizing a conjugate of formula (M-I) or (D-I): 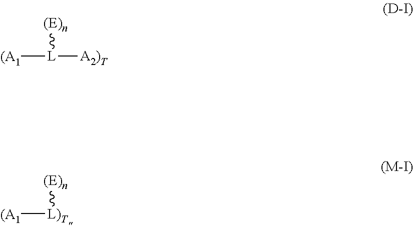
or a pharmaceutically acceptable salt thereof,
wherein each A₁ and each A₂ is independently selected from any one of formulas (A-I)-(A-XIII): 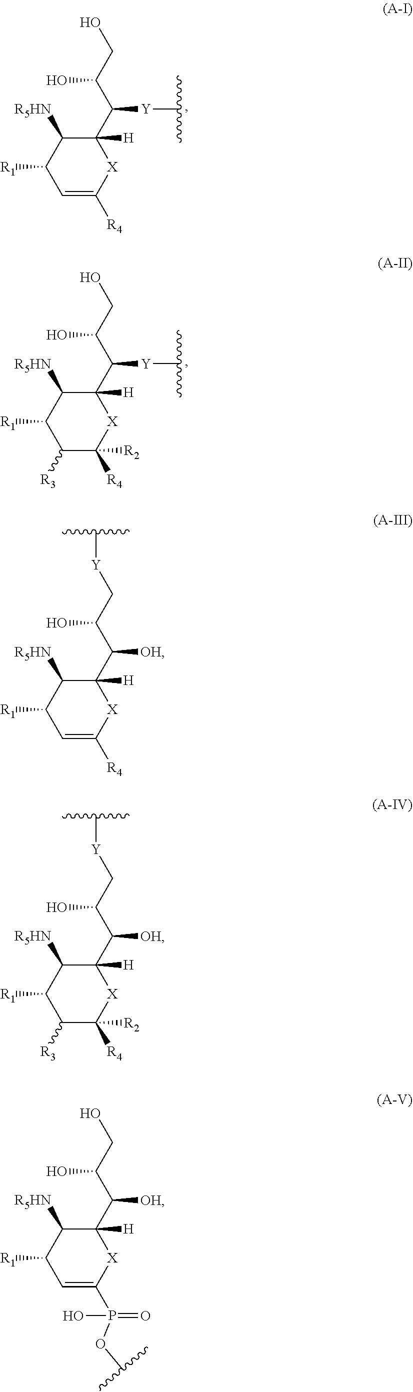 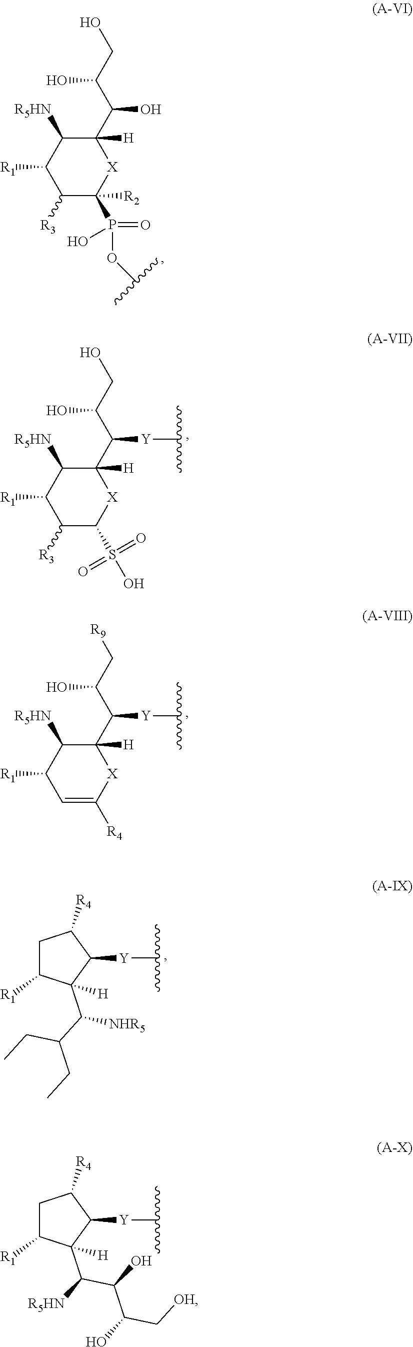 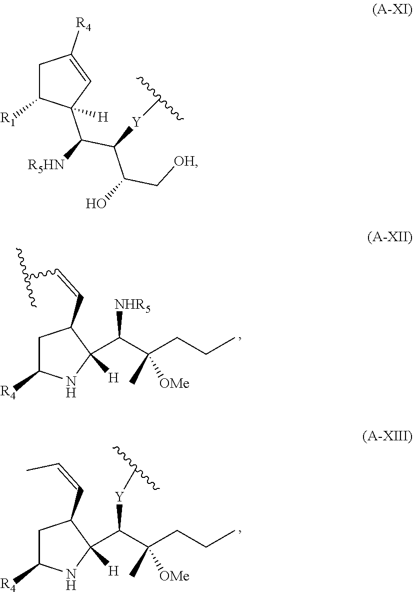
wherein R₁ is selected from —OH, —NH₂, —NHC(═NH)NH₂, and —NHC(═NH)NHR₆;
R₂ and R₃ are each independently selected from —H, —OH, —F, —Cl, and —Br;
R₄ is selected from —CO₂H, —P(═O)(OH)₂, —SO₃H;
R₅ is selected from —COCH₃, —COCF₃, —SO₂CH₃;
X is selected from —O— and —S—;
Y is selected from: 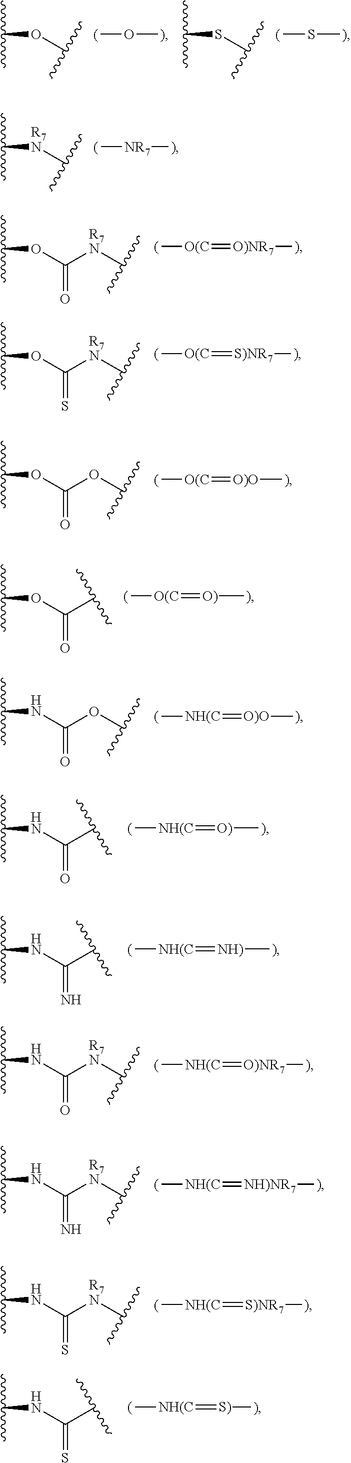 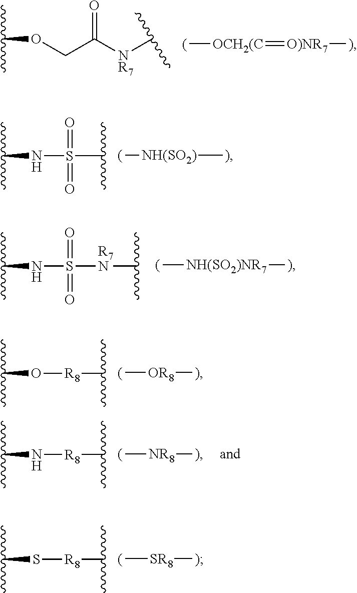
R₆ is selected from 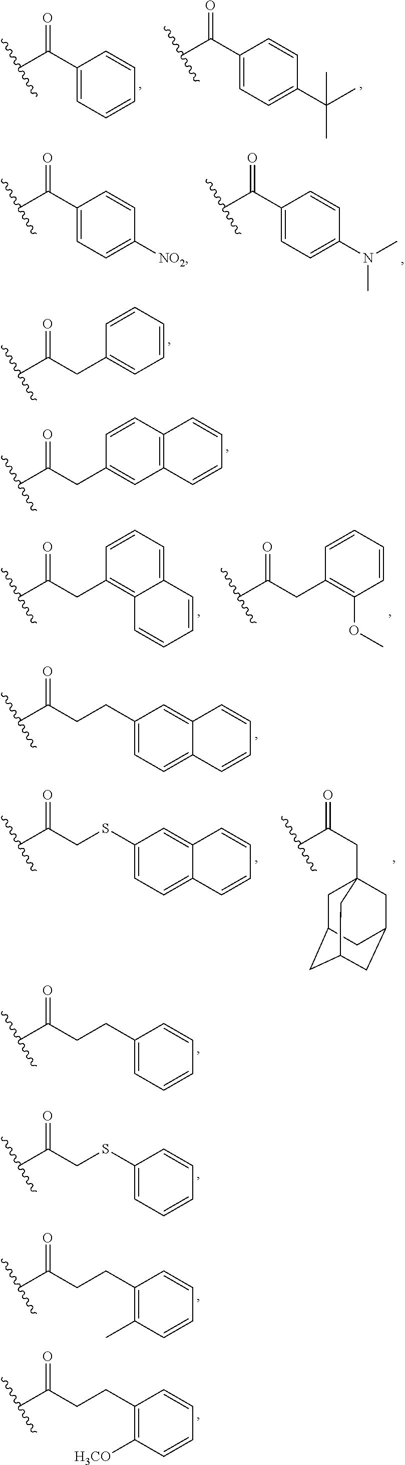 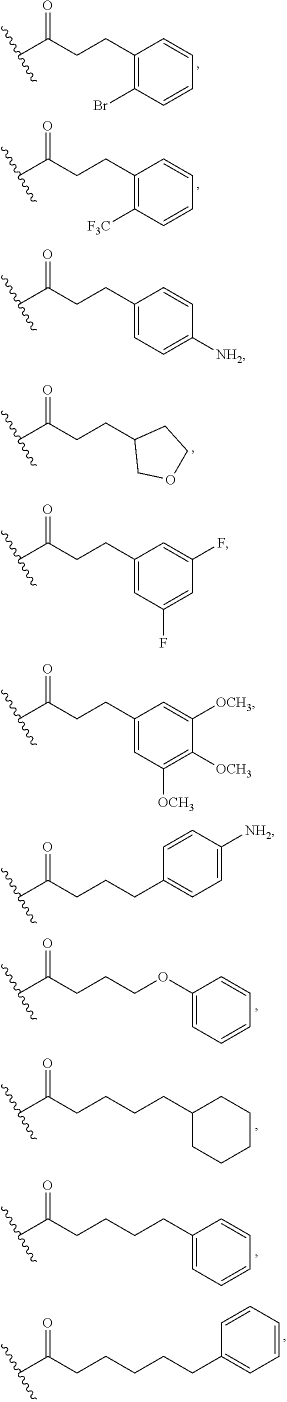 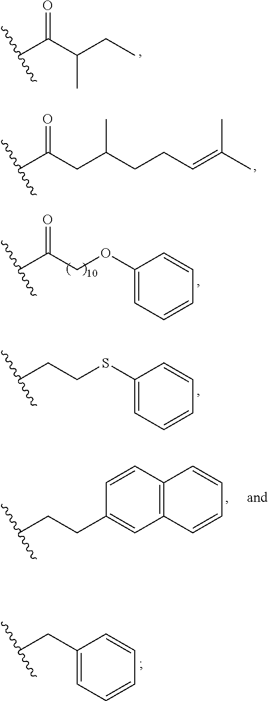
R₇ is selected from H, C1-C20 alkyl, C3-C20 cycloalkyl, C3-C20 heterocycloalkyl; C5-C15 aryl, and C2-C15 heteroaryl;
R₈ is selected from C3-C20 heterocycloalkyl, C5-C15 aryl, and C2-C15 heteroaryl;
R₉ is selected from —H, a halogen (e.g., Cl or F), —OR₁₀, —NHC(═O)R₇, optionally substituted C1-C20 alkyl, C3-C20 cycloalkyl, C3-C20 heterocycloalkyl; C5-C15 aryl, and C2-C15 heteroaryl; and
R₁₀ is selected from C1-C20 alkyl, C3-C20 cycloalkyl, C3-C20 heterocycloalkyl; C5-C15 aryl, and C2-C15 heteroaryl;
n is 1 or 2;
each E comprises an Fc domain monomer or an albumin protein;
L is a linker;
T is an integer from 1 to 20; and
each squiggly line indicates that L is covalently attached to each E,
the method comprising:
(a) providing a first composition comprising formula (D-G3-A) or (M-G3-A) or a salt thereof: 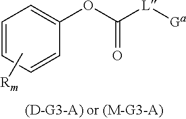
wherein G^{a} is a functional group that reacts with G^{b} to form G;
(b) providing a second composition comprising formula (D-G3-B) or (M-G3-B), or a salt thereof: 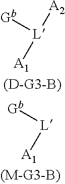
wherein G^{b} is a functional group that reacts with G^{a} to form G;
and
(c) combining the first composition and the second composition to form a first mixture, wherein m is 0, 1, 2, 3, or 4; and each R is, independently, halo, cyano, nitro, optionally substituted C₁-C₆ alkyl group, or optionally substituted C₁-C₆ heteroalkyl group.

40. The method of claim 39, wherein each R is halo.

41. The method of claim 40, wherein each R is, independently, F, Cl, Br, or I.

42. The method of claim 41, wherein each R is F.

43. The method of any one of claims 39-42, wherein m is 1, 2, 3, 4, or 5.

44. The method of claim 43, wherein m is 3 or 4.

45. The method of any one of claims 39-44, wherein m is 3.

46. The method of claim 45, wherein

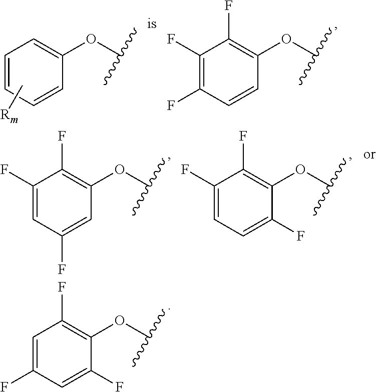

47. The method of claim 46, wherein 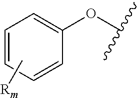
is

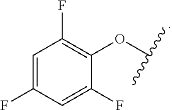 [SMILES: *Oc1c(F)cc(F)cc1F]

48. The method of any one of claims 39-44, wherein m is 4.

49. The method of claim 48, wherein 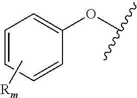
is

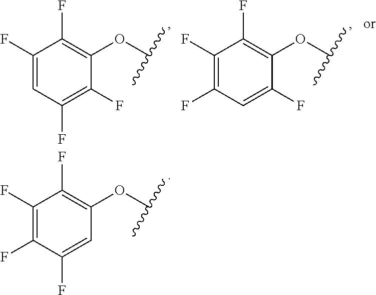

50. The method of claim 49, wherein 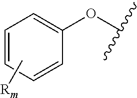
is

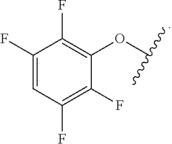 [SMILES: *Oc1c(F)c(F)cc(F)c1F]

51. The method of any one of claims 39-44, wherein 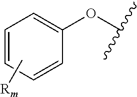
is

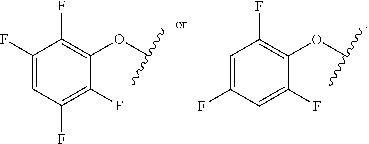 [SMILES: *Oc1c(F)c(F)cc(F)c1F ; *Oc1c(F)cc(F)cc1F]

52. The method of claim 51, wherein 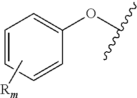
is

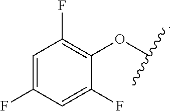 [SMILES: *Oc1c(F)cc(F)cc1F]

53. The method of claim 51, wherein 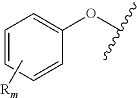
is

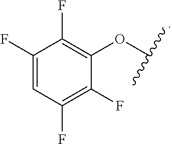 [SMILES: *Oc1c(F)c(F)cc(F)c1F]

54. The method of any one of claims 39-53, wherein step (c) comprises the use of a Cu(I) source.

55. The method of any one of claims 39-54, wherein the method further comprises:
(d) providing a third composition comprising E; and
(e) combing the third composition, the first mixture, and a buffer to form a second mixture.

56. The method of any one of claims 39-55, wherein G^{a} comprises optionally substituted amino.

57. The method of claim 56, wherein G^{b} comprises a carbonyl.

58. The method of any one of claims 39-55, wherein G^{a} comprises a carbonyl.

59. The method of claim 58, wherein G^{b} comprises optionally substituted amino.

60. The method of any one of claims 39-55, wherein G^{a} comprises an azido group.

61. The method of claim 60, wherein G^{b} comprises an alkynyl group.

62. The method of any one of claims 39-55, wherein G^{a} comprises an alkynyl group.

63. The method of claim 62, wherein G^{b} comprises an azido group.

64. The method of any one of claims 1-63, wherein the conjugate of formula (D-I) has the structure:

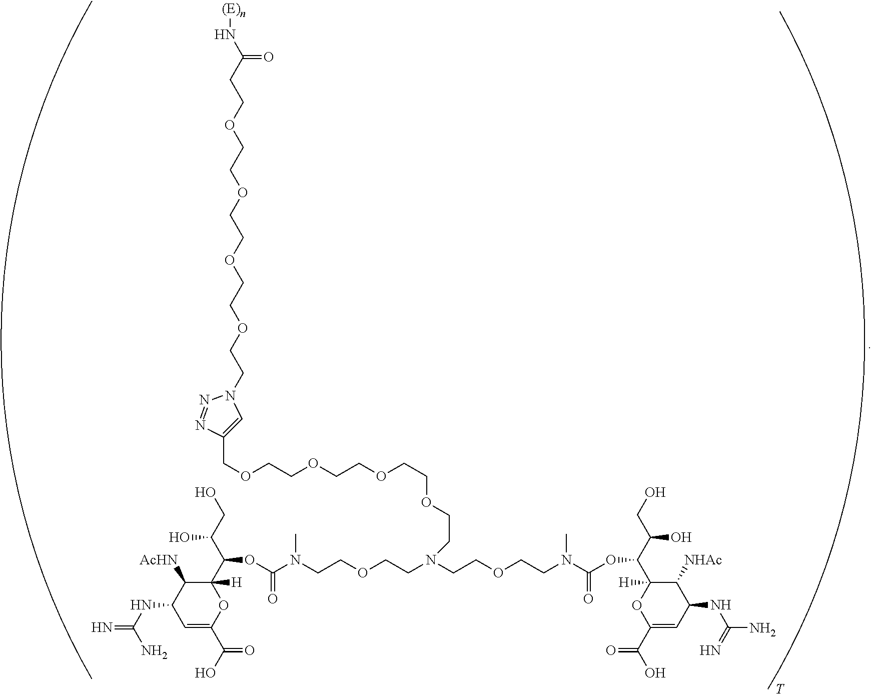

65. The method of any one of claims 1-64, wherein n is 2 and each E is an Fc domain monomer.

66. The method of any one of claims 1-65, wherein each E comprises the amino acid sequence of any one of SEQ ID NOs: 1-14.

67. The method of claim 66, wherein each E comprises the amino acid sequence of any one of SEQ ID NO: 11, SEQ ID NO: 12, SEQ ID NO: 13, or SEQ ID NO: 14.

68. The method of any one of claims 1-64, wherein n is 1 and E is an albumin protein.


SEQUENCE LISTING (14 sequences)

SEQ ID NO: 1 - protein, 247 aa, Artificial Sequence
  Synthetic Construct
  MISC_FEATURE (20)..(20): Xaa is Cys or Ser
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 2 - protein, 247 aa, Artificial Sequence
  Synthetic Construct
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPGK

SEQ ID NO: 3 - protein, 247 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 247 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 247 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 246 aa, Artificial Sequence
  Synthetic Construct
  MISC_FEATURE (20)..(20): Xaa is Cys or Ser
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSXDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 9 - protein, 246 aa, Artificial Sequence
  Synthetic Construct
  MISC_FEATURE (52)..(52): Xaa is Met or Tyr
  MISC_FEATURE (54)..(54): Xaa is Ser or Thr
  MISC_FEATURE (56)..(56): Xaa is Thr or Glu
  MISC_FEATURE (156)..(156): Xaa is Asp or Glu
  MISC_FEATURE (158)..(158): Xaa is Leu or Met
  MISC_FEATURE (228)..(228): Xaa is Met or Leu
  MISC_FEATURE (234)..(234): Xaa is Asn or Ser
NVNHKPSNTKVDKKVEPKSSDKTHTCPPCPAPELLGGPSVFLFPPKPKDTLXIXRXPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVXHEALHXHYTQKSLSLSPG

SEQ ID NO: 10 - protein, 246 aa, Artificial Sequence
  Synthetic Construct
  MISC_FEATURE (156)..(156): Xaa is Asp or Glu
  MISC_FEATURE (158)..(158): Xaa is Leu or Met
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRXEXTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 246 aa, Artificial Sequence
  Synthetic Construct
NVNHKPSNTKVDKKVEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 246 aa, Artificial Sequence
  Synthetic Construct
NVNHKPSNTKVDKKVEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG